(19) United States
(12) Patent Application Publication
(10) Pub. No.: US 2026/0327685 A1
(43) Pub. Date: Oct. 22, 2026

(54) METHOD FOR TRANSCRIPTOME ANALYSIS OF SINGLE CELLS

(71) Applicant: HIFIBIO SAS, Paris (FR)

(72) Inventors: Annabelle Patricia Véronique Gerard, Paris (FR); Kévin Armand Grosselin, Paris (FR); Adeline Frederique Joelle Poitou, Malakoff (FR); Sami Ellouze, Chateney Malabry (FR); Yannick Michel Louis Pousse, Villejuif (FR); Marcel Reichen, Bonaduz (CH); Allan Jensen, Frederiksberg C (DK); Colin Brenan, Marblehead, MA (US)

(21) Appl. No.: 19/648,203

(22) Filed: Apr. 15, 2026

Related U.S. Application Data
(63) Continuation of application No. 16/060,723, filed on Dec. 8, 2016, now Pat. No. 12,630,859.
(60) Provisional application No. 62/264,414, filed on Dec. 8, 2015.

(30) Foreign Application Priority Data
Jun. 28, 2016 (EP) 16305783.9

(51) Int. Cl. C12Q 1/6806

(52) U.S. Cl. CPC C12Q 1/6806

(57) ABSTRACT

The present invention concerns a method for capturing and barcoding nucleic acid from single cells, a plurality of microfluidic droplets and a method for preparing said plurality of microfluidic droplets.

### CROSS-REFERENCE TO RELATED APPLICATIONS

This application is a continuation of U.S. patent application Ser. No. 16/060,723, filed Nov. 18, 2018, which is a national entry under 35 U.S.C. 371 of PCT/EP2016/080341, filed Dec. 8, 2016, which claims priority to European patent application no. 16304783.9 filed Jun. 28, 2016, and priority to U.S. Provisional patent application No. 62/264,414, filed Dec. 8, 2015, all of which are incorporated herein by reference.

### SEQUENCE LISTING

This application contains a Sequence Listing, which has been submitted electronically in xml format and is hereby incorporated by reference in its entirety. Said xml copy, created Apr. 14, 2026 is named B231-0020US01-sequence listing.xml and is 184,320 bytes in size.

The present invention concerns a method for capturing and barcoding nucleic acid from single cells, a plurality of compartments, in particular a plurality of microfluidic droplets and a method for preparing a plurality of microfluidic droplets.

There is a long-felt yet unmet need in the art for rapid isolation and sequencing from one or more transcripts up to the whole transcriptome present in a single cell. The need is particularly significant in the field of antibody drug discovery, immune cell and tumor cell profiling.

Global studies of single cells have been enabled by a tremendous increase in the sensitivity of scientific instruments and an ever-growing automation of all steps from sample preparation to data analysis. In the state of the art, different methods are so far described to capture single cell nucleic acids and to barcode said nucleic acids.

However most of said protocols still require large reaction volumes, or multiple, time consuming steps. High volumes have the disadvantage that they require larger amounts of cost expensive enzymes and nucleotides that are needed for the reverse transcription (RT) of RNAs.

Reducing the reaction volume and in the meantime obtaining a reliable and efficient reverse transcription is therefore an important need in the art.

In the state of the art, different authors addressed this problem and tried to reduce reaction volume in order to increase the throughput.

White et al. (Proc Natl Acad Sci USA. 2011 Aug. 23; 108(34):13999-4004) describe a microfluidic device capable of performing RT-qPCR measurements of gene expression from hundreds of single cells per run, executing single-cell processing, including cell capture, cell lysis, reverse transcription, and quantitative PCR. However, incited to try smaller reaction volumes, White et al. (Proc Natl Acad Sci USA. 2011 Aug. 23; 108(34):13999-4004) performed 300 parallel RT-qPCR and demonstrated that RT is inhibited in volumes that are smaller than 5 nL in the reaction conditions tested. The researchers thus performed the RT reaction in 67 nL per cell and further claimed that the combination of RT and qPCR in a single reaction precludes large-scale transcriptome analysis and/or unbiased amplification.

Furthermore, a method using a simple axisymmetric flow-focusing device has been described for single cell mRNA capture by DeKosky et al. (Nat Med. 2015 January; 21(1):86-91.). The RT and PCR reaction of the method take place in an emulsion. This process has proven to be efficient, yet requiring performing sequential 3 steps process.

Along the same line, Eastburn et al. (Anal Chem. 2013 Aug. 20; 85(16):8016-21) performed RT in small droplet volume, yet in a time consuming 3-4 steps process. They mentioned also RT inhibition in small volume from cell proteinase. To overcome this problem, they treat the cells with proteinase, dilute cell lysate, split drops, and pico-inject RT-PCR reagents.

Rotem et al. (PLoS One. 2015 May 22; 10(5):e0116328) encapsulate oligo in droplet population and fuse them to droplet containing cells while pico-injecting the RT enzymes and buffers. This 3 step process produces ~100 pL droplets allowing encapsulation of 100.000 cells in 3 h. Although this method relies on single cell cDNA labeling, the transcriptomic sequence data comes from an aggregate of multiple phenotypically and genotypically uncorrelated cells.

Macosko et al. (Cell. 2015 May 21; 161(5):1202-14) used droplet-based microfluidics to encapsulate cells together with lysis reagents and barcoded beads to capture mRNA in 1 nL drops. The beads are then recovered to perform, off chip, the conversion of the mRNA captured on beads into cDNA. The third step is then devoted to library preparation and amplification.

The international patent application WO2015/164212 refers to a method for encapsulating and barcoding single cell nucleic acids. WO2015/164212 discloses that RT of mRNA is strongly inhibited in volumes that are smaller than 3 nL (Example 4 of WO2015/164212 and Georgio G. et al. Nat Biotechnol. 2014 February; 32(2):158-68).

Contrary to this, the inventors succeeded in developing a single-step, reverse transcription of single cell mRNA wherein barcoded cDNA is produced in volumes smaller than 3 nL. This method consists of a single step to obtain the cDNA and has an increased throughput (factor 5-10 times; 100.000 cells encapsulated with barcoded primers in 1 h) and reduced costs due to the reduced volume. The inventors thus established experimental conditions leading to efficient cDNA synthesis in micro reactors having a volume of less than 3 nL.

### DETAILED DESCRIPTION OF THE INVENTION

Unless otherwise defined, scientific and technical terms used in connection with the disclosure shall have the meanings that are commonly understood by those of ordinary skill in the art.

Further, unless otherwise required by context, singular terms shall include pluralities and plural terms shall include the singular.

Generally, nomenclatures utilized in connection with, and techniques of, cell and tissue culture, molecular biology, and protein and oligo- or polynucleotide chemistry and hybridization described herein are those well-known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The practice of the disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “nucleic acid” as herein used generally refers to at least one molecule or strand of DNA, RNA, miRNA or a derivative or mimic thereof, comprising at least one nucleobase, such as, for example, a naturally occurring purine or pyrimidine base found in DNA (e.g., adenine “A,” guanine “G,” thymine “T,” and cytosine “C”) or RNA (e.g. A, G, uracil “U,” and C). The term “nucleic acid” encompasses the term “oligonucleotide”.

“RNA” herein refers to functional RNA, such as, without limitation to the examples, mRNA, tRNA, rRNA, catalytic RNA, siRNA, miRNA, antisense RNA, lncRNA and piRNA.

As it will be understood by those skilled in the art, the depiction of a single strand also defines the sequence of the complementary strand. Thus, a nucleic acid also encompasses the complementary strand of a depicted single strand. The term nucleic acid thus encompasses complementary DNA. As it will also be appreciated by those skilled in the art, many variants of a nucleic acid may be used for the same purpose as a given nucleic acid. Thus, a nucleic acid also encompasses substantially identical nucleic acids and complements thereof. As it will also be understood by those skilled in the art, a single strand nucleic acid, such as, a primer, may hybridize to the target sequence under hybridization conditions, preferably stringent hybridization conditions. Thus, a nucleic acid also encompasses a primer that hybridizes under hybridization conditions to a target sequence.

The term “oligonucleotide” refers to at least one molecule of about 3 to about 200 nucleobases in length.

These definitions refer to at least one single-stranded molecule, but in some embodiments encompass also at least one additional strand that is partially, substantially or fully complementary to the at least one single-stranded molecule. Accordingly, in some embodiments said definitions refer to double stranded molecules.

Thus, in one embodiment, a nucleic acid refers to at least one double-stranded molecule that comprises one or more complementary strand(s) or “complement(s)” of a particular sequence comprising a strand of the molecule.

“Gene” as used herein may be a genomic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA, antisense RNA, lncRNA and piRNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

The term “stringent condition” or “high stringency condition” as used herein corresponds to conditions that are suitable to produce binding pairs between nucleic acids having a determined level of complementarity, while being unsuitable to the formation of binding pairs between nucleic acids displaying a complementarity inferior to said determined level. Stringent conditions are the combination of both hybridization and wash conditions and are sequence dependent. These conditions may be modified according to methods known from those skilled in the art (Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, high stringency conditions are selected to be about 5° C. lower than the thermal melting point (Tm), preferably at a temperature close to the Tm of perfectly base-paired duplexes (Andersen, Nucleic acid Hybridization, Springer, 1999, p. 54). Hybridization procedures are well known in the art and are described for example in Ausubel, F. M., Brent, R., Kingston, R. E., Moore, D. D., Seidman, J. G., Smith, J. A., Struhl, K. eds. (1998) Current protocols in molecular biology. V. B. Chanda, series ed. New York: John Wiley & Sons.

High stringency conditions typically involve hybridizing at about 50° C. to about 68° C., wherein said temperature typically corresponds to the highest melting temperature T_{M} of the nucleic acid to be hybridized with a target sequence, for example, in 5×SSC/5×Denhardt's solution/1.0% SDS, and washing in 0.2×SSC/0.1% SDS at about 60° C. to about 68° C.

For instance, in context with the present invention the primer sequence comprised in the oligonucleotide typically hybridizes with a complementary nucleic acid, for example a complementary RNA sequence, cDNA or DNA sequence, at about 50° C. to about 68° C. in a compartment (in particular in a droplet), or in a plurality of compartments (in particular in a plurality of droplets) further comprising a lysing composition and a reverse transcription composition, as defined herein below.

The term “antibody” refers to immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants of antibodies, including derivatives such as humanized antibodies. In certain conventional antibodies, two heavy chains are linked to each other by disulfide bonds and each heavy chain is linked to a light chain by a disulfide bond. There are two types of light chain, lambda (A) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain.

The specificity of the antibody resides in the structural complementarity between the antibody combining site and the antigenic determinant. Antibody combining sites are made up of residues that are primarily from the hypervariable or complementarity determining regions (CDRs). Occasionally, residues from non hypervariable or framework regions (FR) influence the overall domain structure and hence the combining site. Complementarity determining regions (CDRs) refer to amino acid sequences which, together, define the binding affinity and specificity of the natural Fv region of a native immunoglobulin binding-site. The light and heavy chains of an immunoglobulin each have three CDRs, designated L-CDR1, L-CDR2, L-CDR3 and H-CDR1, H-CDR2, H-CDR3, respectively. Therefore, an antigen-binding site includes six CDRs, comprising the CDR set from each of a heavy and a light chain V region. Framework Regions (FRs) refer to amino acid sequences interposed between CDRs, i.e. to those portions of immunoglobulin light and heavy chain variable regions that are relatively conserved among different immunoglobulins in a single species, as defined by Kabat, et al. (Sequences of Proteins of Immunological Interest (National Institutes of Health, Bethesda, Md., 1991).

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The term antibody further denotes single chain antibodies, for instance Camelidae antibodies, or nanobodies or V_{HH}.

The term “T-cell receptor” herein refers to an antigen-recognition molecule present on the surface of T cells (i.e., T lymphocytes). This definition expressly includes the understanding of the term as known in the art, and includes, for example, a receptor that comprises or consists of a disulfide-linked heterodimer of the highly variable alpha or beta chains expressed at the cell membrane as a complex with the invariant CD3 chains, or a receptor that comprises or consists of variable gamma and delta chains expressed at the cell membrane as a complex with CD3 on a subset of T-cells.

“Antibody genes” and “T-cell receptor genes” undergo a unique mechanism of genetic recombination, called V(D)J recombination, that occurs only in developing lymphocytes during the early stages of T and B cell maturation. It further involves somatic recombination, and results in the highly diverse repertoire of antibodies/immunoglobulins (Igs) and T cell receptors (TCRs) found on B cells and T cells, respectively.

### Method for Capturing and Barcoding Single Cell Nucleic Acid

“Capturina” herein refers to hybridizing single cell nucleic acid(s) to primer(s), in a compartment, in particular in a droplet, or in a plurality of compartments, in particular in a plurality of droplets.

“Barcodinq” herein refers to adding a genetic sequence, a so-called barcode sequence as further defined herein below, to a nucleic acid which allows distinguishing said barcoded nucleic acid from a nucleic acid having another added genetic sequence, i.e. another unique barcode sequence.

The inventors have developed a single-step, reverse transcription of single cell mRNA, wherein a single cell is captured in volumes less than 3 nL, and preferably in sub-nanoliter volumes of a droplet, and barcoded cDNA is produced with an increased throughput (factor 5-10 times; 100.000 cells encapsulated with barcoded primers in 1 h) and reduced costs.

The inventors established experimental conditions leading to efficient cDNA synthesis micro reactors of less than 3 nL, independent of cell size and shape.

Accordingly, the present invention refers to a method for capturing and barcoding single cell nucleic acid comprising:
a) providing a plurality of cells contained within a plurality of compartments, at least some of the compartments comprising a single cell, a reverse transcriptase and at least one type of an oligonucleotide, wherein the at least one type of oligonucleotide comprises a barcode sequence and a primer sequence, wherein each different primer sequence defines a different oligonucleotide type, and wherein the compartments of the plurality of the compartments contain one or more barcode sequences distinguishable from barcode sequences contained in other compartments of the plurality of compartments;
b) lysing at least some of the cells within the compartments to release nucleic acids from the cells;
c) hybridizing at least some of the released nucleic acids to said oligonucleotide in at least some of the compartments;
d) reverse transcribing the released nucleic acids hybridized to said oligonucleotide using the primer sequence in at least some of the compartments;
wherein the plurality of compartments has a volume of less than 3 nL; and
preferably wherein the concentration of each type of oligonucleotide in the microfluidic droplets is at least 100 nM.

“Compartment”, also referred to as “container”, herein refers, for example, to a plate, well, tube, channel, nano well, nano drop, nano tube, or nano channel.

In a preferred embodiment in context of the invention, a compartment is a droplet, more preferably a microfluidic droplet.

Accordingly, in a preferred embodiment, a plurality of compartments is a plurality of droplets, more preferably a plurality of microfluidic droplets.

In some embodiments, the at least one type of oligonucleotide is introduced into the compartments by initially binding the at least one type of oligonucleotide to a particle, then subsequently releasing them from the particle after the particle has been incorporated into a compartment.

In certain embodiments of the invention, binding the at least one type of oligonucleotide initially to the particle facilitates the introduction of only one type of oligonucleotide into each compartment.

According to the above, in one embodiment, step a) further comprises providing a plurality of particles contained within said plurality of compartments, and wherein at least some of the compartments further comprise a particle.

In a related embodiment, the at least one type of an oligonucleotide is bound to said particle.

Accordingly, in a preferred embodiment, the invention refers to a method for capturing and barcoding single cell nucleic acid comprising:
a) providing a plurality of cells and a plurality of particles contained within a plurality of compartments, at least some of the compartments comprising a single cell, a reverse transcriptase and a particle comprising at least one type of an oligonucleotide optionally bound thereto, wherein the at least one type of oligonucleotide comprises a barcode sequence and a primer sequence, wherein each different primer sequence defines a different oligonucleotide type, and wherein the compartments of the plurality of compartments contain one or more barcode sequences distinguishable from barcode sequences contained in other compartments of the plurality of compartments;
b) lysing at least some of the cells within the compartments to release nucleic acids from the cells;
c) hybridizing at least some of the released nucleic acids to said oligonucleotide in at least some of the compartments;
d) reverse transcribing the released nucleic acids hybridized to said oligonucleotide using the primer sequence in at least some of the compartments;
wherein the plurality of compartments has a volume of less than 3 nL; and
preferably wherein the concentration of each type of oligonucleotide in the compartments containing a particle is at least 100 nM.

The “particle” in context of the present invention refers to a microparticle.

In one embodiment the particle is a hydrogel particle, a polymeric particle or a magnetic particle.

The particle may have irregular or regular shape. For example, the particle can be spherical, ellipsoidal, or cubic.

In one embodiment, the particle(s) and the cell(s) may be introduced into the compartment(s) or encapsulated within the compartments simultaneously or sequentially, in any suitable order.

In one preferred embodiment, the particle(s) and the cell(s) may be encapsulated within the droplet(s) simultaneously or sequentially, in any suitable order.

In some embodiments, the particle in context of the present invention is a hydrogel particle.

“Hydroqel particles” are for example described in the International Patent Application No. WO 2008/109176, entitled “Assay and other reactions involving droplets”. Examples of hydrogels include, but are not limited to agarose, poly(ethylene glycol) diacrylate, or acrylamide-based gels, such as bis-acrylamide, polyacrylamide, streptavidine acrylamide, poly-N-isopropylacrylamide, or poly N-isopropylpolyacrylamide or mixtures thereof. In one example the hydrogel particle comprises acrylamide, bis-acrylamide and strepatvidine acrylamide.

For example, an aqueous solution of a monomer may be dispersed in a compartment, for instance a droplet, and then polymerized, e.g., to form a gel. Another example is a hydrogel, such as alginic acid that can be gelled by the addition of calcium ions. In some cases, gelation initiators (ammonium persulfate and TEMED for acrylamide, or Ca²⁺ for alginate) can be added to a compartment, for instance a droplet, for example, by co-flow with the aqueous phase, by co-flow through the oil phase, or by coalescence of two different drops, e.g., as discussed in U.S. patent application Ser. No. 11/360,845, filed Feb. 23, 2006, entitled “Electronic Control of Fluidic Species,” by Link, et ah, published as U.S. Patent Application Publication No. 2007/000342 on Jan. 4, 2007; or in U.S. patent application Ser. No. 11/698,298, filed Jan. 24, 2007, entitled “Fluidic Droplet Coalescence,” by Ahn, et al.; each incorporated herein by reference in their entireties.

In another set of embodiments, the particles may comprise one or more polymers and are thus herein referred to as “polymeric particle”. Exemplary polymers include, but are not limited to, polystyrene (PS), polycaprolactone (PCL), polyisoprene (PIP), poly(lactic acid), polyethylene, polypropylene, polyacrylonitrile, polyimide, polyamide, and/or mixtures and/or co-polymers of these and/or other polymers.

In addition, in some embodiments, the particles may be magnetic and is thus referred to as “magnetic particle”, which could allow for the magnetic manipulation of the particles. For example, the particles may comprise iron or other magnetic materials. The particles could also be functionalized so that they could have other molecules attached, such as proteins, nucleic acids or small molecules. Thus, some embodiments of the present invention are directed to a set of particles defining a library of, for example, nucleic acids, proteins, small molecules, or other species such as those described herein. In some embodiments, the particle may be fluorescent.

In one embodiment, the particle comprises streptavidin. Streptavidin may be coupled to the surface of the particle defined herein above.

In one embodiment the hydrogel particles have a size from 1 pL to 1000 pL, such as 1 pL to 500 pL, 1 pL to 400 pL, 1 pL to 400 pL, 1 pL to 300 pL, for example 5 pL to 300 pL, 5 pL to 250 pL, 5 pL to 200 pL, 10 pL to 250 pL, 10 pL to 200 pL, preferably 10 pL to 200 pL.

As it will be understood by the skilled in the art and as it will be further explained in the section “A method for preparing a plurality of microfluidic droplets” the number of cells encapsulated in one compartment, for instance a droplet, follows a probability distribution, for example a Poisson distribution, and depends on, for example, the concentration of the cells in the first fluid, the concentration of particles in the second fluid, the geometry of the main channel and the secondary channel, the injection parameters of the first fluid, of the second fluid and of the carrier fluid used in the method for preparing a plurality of microfluidic droplets of the invention.

Accordingly, in step a) of the method of the invention, a plurality of cells comprised in an aqueous composition are introduced into a plurality of compartments, in particular encapsulated in a plurality of microfluidic droplets, and the number of cells introduced into 1 compartment, in particular encapsulated in 1 droplet follows, depending on the parameters used in the “method for preparing a plurality of microfluidic droplets” a Poisson distribution. The parameters can be adapted to obtain, for instance, compartments with either 1 or 0 cells in it, thus avoiding compartments containing several cells.

As shown by the inventors, the parameters used to introduce into or encapsulate, preferably encapsulate, cells can be adapted to obtain at least some of the compartments comprising a single cell. The at least some compartments further comprise, in context of the present invention, a reverse transcriptase and at least one type of an oligonucleotide, as further defined herein below.

Accordingly in one embodiment, the at least some compartments of step a), preferably droplets of step a), are provided at a velocity of 1 to 2000 compartments per second, such as 1 to 1000 compartments per second, 1 to 800 compartments per second, 1 to 700 compartments per second, 1 to 600 compartments per second, 1 to 500 compartments per second, 1 to 400 compartments per second, 1 to 300 compartments per second, 1 to 200 compartments per second, 1 to 100 compartments per second, 1 to 80 compartments per second, 1 to 70 compartments per second, 1 to 50 compartments per second, for example 10 to 300 compartments per second, 50 to 300 compartments per second, 100 to 300 compartments per second, 150 to 300 compartments per second, 150 to 250 compartments per second, 175 to 250 compartments per second, typically, 1 to 1000 compartments per second, preferably 175 to 250 compartments per second.

Furthermore, as it will be understood by the skilled in the art, based on different parameters, compartments can be produced in a way that they contain 0, 1 or 2 particles per compartment, thus avoiding compartments containing more than two particles. In one particular embodiment, the compartments are produced in a way that they contain 0 or 1 particle per compartment, thus avoiding compartments containing more than one particle.

In a particular example, as it will be understood by the skilled in the art, based on different parameters, such as, for example, particle concentration and flow velocities the droplets can be produced in a way that they contain 0, 1 or 2 particles per droplet, thus avoiding droplets containing more than two particles. In one particular embodiment, the droplets are produced in a way that they contain 0 or 1 particle per droplet, thus avoiding droplets containing more than one particle.

Accordingly, in one embodiment, the particles are introduced into or encapsulated within the compartments at no more than about 2 particles/compartments, preferably, in a further embodiment, the particles are introduced into or encapsulated within the compartments at no more than about 1 particle/compartment, or the particles are introduced into or encapsulated within the compartments preferentially with 1 particle/compartment, or the particles are introduced into or encapsulated within the compartments with an average of 1 particle/compartment.

In one embodiment, in particular when referring to droplets as compartments, the particles are preferably encapsulated within the droplets, with the number of particles as defined herein above. In line with the above, it will be also understood by the skilled in the art that, when referring to droplets, the size of the droplets as provided in step a) follows a probability distribution, such as a Poisson distribution. It will be further understood that the parameters used in step a) for providing microfluidic droplets wherein at least some of those droplets are as defined in the claim can be regulated in order to obtain a plurality of microfluidic droplets having a specific volume.

A “droplet” generally refers to a measure of volume and further refers in context of the present invention, to an isolated portion of a first fluid that is surrounded by a second fluid. It is to be noted that a droplet is not necessarily spherical, but may assume other shapes as well, for example, depending on the external environment.

By “the plurality of compartments has a volume of less than 3 nL”, it is meant that each compartment, in the plurality of compartments, has a volume of less than 3 nL.

The “compartment” or “the plurality of compartments”, preferably the “droplet” or “the plurality of droplets” in context of the invention has a volume of less than 3 nL. In one embodiment, said plurality of microfluidic droplets has a volume of less than 2.5 nL, less than 2 nL, less than 1.5 nL, less than 1 nL, less than 0.5 nL, for example 0.1 nL to 3 nL, 0.5 nL to 3 nL, 1 nL to 3 nL, typically, 0.1 nL, 0.5 nL, 1 nL, 1.2 nL, 1.4 nL, 1.6 nL, 1.8 nL, 2.0 nL, 2.2 nL, 2.4 nL, 2.6 nL, 2.8 nL, 3 nL.

In one preferred embodiment, the plurality of compartments has a volume equal to or less than 1 nL.

Contrary to prior art disclosure the inventors demonstrated that the method of the present invention can be used to capture and barcode efficiently the whole transcriptome of a single cell (as demonstrated in Example 10) or, alternatively, to specifically capture and barcode a gene specific transcriptome in volumes less than 3 nL provided the concentration of one type of an oligonucleotide as defined herein above is 100 nM or more.

The “transcriptome” generally refers to the set of all or a part of all messenger RNA molecules in one cell or a population of cells. Accordingly, the “transcriptome of a cell” or “the transcriptome of a single cell” herein refers to the set of all or a part of all messenger RNA molecules in one cell.

As it will be understood by the skilled in the art, a gene specific transcriptome thus refers to the set of all messenger RNA molecules derived from one gene.

As it is known by the skilled in the art, different gene products, so called isoforms, may be encoded by one gene. Accordingly, a gene specific transcriptome may further refer to the messenger RNA molecules of at least one specific isoform of one specific gene, such as the messenger RNA molecules of 1, 2, 3, or 4 specific isoforms of one specific gene or to the messenger RNA molecules of all isoforms of one specific gene.

Accordingly, in one embodiment, the nucleic acid in context of the present invention is cDNA or RNA, preferably RNA.

In one embodiment, RNA is selected from the group consisting of mRNA, tRNA, rRNA, catalytic RNA, siRNA, miRNA, antisense RNA, lncRNA and pIRNA, preferably mRNA.

In a further embodiment, mRNA comprises a poly A sequence, also called poly A tail.

Furthermore, in one particular embodiment, the “at least some of the released nucleic acids” in context of the present invention refers to at least one nucleic acid, preferably, at least 2, at least 3, at least 4, at least 5, nucleic acids or more. In one particular, the nucleic acids of step c) and d) refer to 1, 2, 3, 4, 5, 6, 7, 8, 9 or 10 nucleic acids.

The method of the invention can be used to transcribe from one specific nucleic acid up to all nucleic acids of a single cell.

Accordingly, in further embodiments, the “at least some of the released nucleic acids” of step c) and d) in context of the present invention refers to from 1 to 100000 nucleic acids, such as 1 to 80000, 1 to 60000, 1 to 40000, such as 1, 1000, 2000, 4000, 6000, 8000, 10000, 12000, 14000, 16000, 20000, 25000, 30000, 45000, 50000 nucleic acids. Accordingly, in one embodiment, the nucleic acid herein refers to the RNA of all genes.

As mentioned above, as demonstrated by the inventors the method of the invention also refers to capturing and transcribing a gene specific transcriptome.

Accordingly, in one embodiment, the nucleic acid herein refers to the RNA of (a) specific gene(s). As it will be understood by the skilled in the art said gene(s) may be any gene of interest.

In one embodiment, said specific gene is selected from the group consisting of antibody heavy chain variable gene, antibody heavy chain constant gene, antibody light chain variable gene, antibody light chain constant gene, alpha T-cell receptor gene, beta T-cell receptor gene, and delta T-cell receptor gene and gamma T-cell receptor gene.

In one embodiment the nucleic acid as referred to in step c) and d) is a gene specific nucleic acid wherein the gene may be selected from the list consisting of antibody heavy chain variable gene, antibody heavy chain constant gene, antibody light chain variable gene, antibody light chain constant gene, alpha T-cell receptor gene, beta T-cell receptor gene, and delta T-cell receptor gene and gamma T-cell receptor gene.

In one particular embodiment the nucleic acid referred to in step c) and d) is a nucleic acid specific to at least two genes, wherein the at least two genes are selected from the list consisting of antibody heavy chain variable gene, antibody heavy chain constant gene, antibody light chain variable gene, antibody light chain constant gene, alpha T-cell receptor gene, beta T-cell receptor gene, and delta T-cell receptor gene and gamma T-cell receptor gene.

In one embodiment the nucleic acid as referred to in step c) and d) is a gene specific nucleic acid wherein the gene may be selected from the list consisting of antibody heavy chain variable gene, antibody heavy chain constant gene, antibody light chain variable gene, antibody light chain constant gene, alpha T-cell receptor gene, beta T-cell receptor gene, and delta T-cell receptor gene and gamma T-cell receptor gene, preferably antibody heavy chain variable gene, antibody heavy chain constant gene, antibody light chain variable gene and antibody light chain constant gene.

In a further particular embodiment the nucleic acid referred to in step c) and d) is a nucleic acid specific to at least three genes, wherein the at least three genes are selected from the list consisting of antibody heavy chain variable gene, antibody heavy chain constant gene, antibody light chain variable gene, antibody light chain constant gene, alpha T-cell receptor gene, beta T-cell receptor gene, and delta T-cell receptor gene, preferably, alpha T-cell receptor gene, beta T-cell receptor gene, and delta T-cell receptor gene and gamma T-cell receptor gene.

It will be understood by the skilled in the art that when the transcriptome of a cell is captured, oligonucleotides having a primer sequence, wherein said primer sequence is specific to all mRNAs will be used, such as poly T primer sequences, as further defined herein below, whereas, when a gene specific transcriptome is captured and to be barcoded, oligonucleotides comprising a gene specific primer sequence are used, as further defined herein below.

“At least one type of an oligonucleotide” as used in context of the present invention refers to an oligonucleotide, as defined herein above, comprising a barcode sequence and a primer sequence, wherein each different primer sequence defines a different oligonucleotide type. In one embodiment, the at least one type of an oligonucleotide comprises from 5′ to 3′ a barcode sequence and a primer sequence.

“At least one” in the wording “at least one type of an oliqonucleotide” refers to at least 2, at least 3, at least 4, at least 5, at least 6, at least 7, at least 8, at least 9 at least 10 types or more of an oligonucleotide. The number of different types of oligonucleotides present in one compartment depends on the number of genes of which the RNA is to be captured and barcoded.

As mentioned above, one type of an oligonucleotide distinguishes from another type of an oligonucleotide by its primer sequence.

The whole transcriptome may be transcribed using one primer sequence, the polydT primer. It will be understood, that the number of types of oligonucleotides corresponds to at least the number of specific genes of which the transcriptome is to be captured and barcoded.

Accordingly, in one further embodiment, at least one type of an oligonucleotide refers to 1 to 100 types of an oligonucleotide, 1 to 80, 1 to 60, 1 to 40, 1 to 30, 1 to 20, 1 to 10, preferably, 1, 2, 3, 4, 5, 6, 7, 8, 9 types of an oligonucleotide.

Accordingly, in one embodiment, different types of oligonucleotides bound to the particle contained in one compartment comprise the same barcode sequence.

In one embodiment, when the compartment is a droplet the particle is encapsulated in said droplet.

In one embodiment, different types of oligonucleotides bound to the particle encapsulated in one compartment comprise the same barcode sequence.

As defined in the methods of the invention preferably the concentration of each type of oligonucleotide in the compartment is at least 100 nM.

Accordingly, in some embodiments, the concentration of each type of oligonucleotide in the compartment is at least 100 nM, or preferably more than 100 nM.

In some embodiments, the concentration of each type of oligonucleotide in the compartments is at least 150 nM, at least 200 nM, at least 300 nM, at least 400 nM, at least 500 nM, at least 600 nM, at least 700 nM, at least 800 nM, at least 900 nM and at least 1 μM, such as for instance 100 nM to 5 μM, 100 nM to 4 μM, 100 nM to 3 μM, 100 nM to 2 μM, 100 nM to 1 μM, preferably 100 nM to 500 nM.

In one example, the primer sequence is a poly T primer sequence and the concentration of the oligonucleotide is 100 nM to 3300 nM (corresponding to 3.3 μM).

In a further example, the primer sequence is a gene specific primer sequence and the concentration of the oligonucleotide is 100 nM or 1000 nM (corresponding to 1 μM).

For instance, the inventors demonstrate that the oligonucleotide density/primer concentration per cell, related to compartment volume enable efficient capture and barcoding (with the hypothesis of 1 particle per compartment).

TABLE 1
relation oligonucleotide density/primer concentration
(one type of oligonucleotide containing one given barcode
sequence and one given primer and compartment volume)

| Oligonucleotide | Primer concentration per cell | | | | |
|---|---|---|---|---|---|
| density | in 100 pL | in 500 pL | in 1 nL | in 5 nL | in 10 nL |
| 10e9 | 16.6 μM | 3.3 μM | 1.66 μM | 332 nM | 166 nM |
| 10e8 | 1.66 μM | 332 nM | 166 nM | | |
| 10e7 | 166 nM | | | | |
| 10e6 | | | | | |
| 10e5 | | | | | |

The “barcode sequence” or simply called “barcode” herein refers to a unique nucleic acid sequence that can be distinguished by its sequence from another nucleic acid sequence, thus permitting to uniquely label a nucleic acid sequence so that it can be distinguished from another nucleic acid carrying another barcode sequence.

In one embodiment, the barcode sequence uniquely identifies the nucleic acids released by a single cell from nucleic acids released from other cells, for instance, even after the nucleic acids are pooled together.

In some embodiments, the barcode sequence may be used to distinguish tens, hundreds, or even thousands of nucleic acids, e.g., arising from different cells or other sources.

In one embodiment, the barcode sequence may be of any suitable length. The barcode sequence is preferably of a length sufficient to distinguish the barcode sequence from other barcode sequences. In one embodiment, a barcode sequence has a length of 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 72, 74, 76, 78, 80, 85, 90 or more nucleotides, such as 50 to 85, 60 to 80, 70 to 80 nucleotides.

In one embodiment, the barcode sequence consists of more than one barcode sequence, wherein the barcoded sequences are different.

In a related embodiment, the different barcode sequences may be taken from a “pool” of potential barcode sequences. If the barcode sequence consists of more than one barcode sequence, the barcode sequences may be taken from the same, or different pools of potential barcode sequences. The pool of sequences may be selected using any suitable technique, e.g., randomly, or such that the sequences allow for error detection and/or correction, for example, by being separated by a certain distance (e.g., Hamming distance) such that errors in reading of the barcode sequence can be detected, and in some cases, corrected. The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, or at least 1,000,000 barcode sequences.

Methods to join different barcode sequences taken from one “pool” or more than one pool are known to the skilled in the art and include, but are not limited to, the use of ligases and/or using annealing or a primer extension method.

Non-limiting examples of ligases include DNA ligases such as DNA Ligase I, DNA Ligase II, DNA Ligase Ill, DNA Ligase IV, T4 DNA ligase, T7 DNA ligase, T3 DNA Ligase, E. coli DNA Ligase, Taq DNA Ligase, or the like. Many such ligases may be purchased commercially.

In one embodiment, the barcode sequence is a double stranded or single stranded nucleic acid.

A “primer sequence” is typically a short single-stranded nucleic acid, of between 10 to 50 nucleotides in length, designed to perfectly or almost perfectly match a nucleic acid of interest, to be captured and then amplified by typically PCR or reverse transcribed by typically RT. The primer sequences are “specific” to the nucleic acids they hybridize to, i.e. they preferably hybridize under stringency hybridization conditions, more preferably under high stringency hybridization conditions, or are complementary to or almost complementary to the nucleic acids they hybridize to, also called target sequence.

Typically, the primer sequence serves as a starting point for nucleic acid synthesis, allowing polymerase enzymes such as nucleic acid polymerase to extend the primer sequence and replicate the complementary strand. A primer sequence may be complementary to and hybridize to a target nucleic acid. In some embodiments, a primer sequence is a synthetic primer sequence. In some embodiments, a primer sequence is a non-naturally-occurring primer sequence. A primer sequence typically has a length of 10 to 50 nucleotides. For example, a primer sequence may have a length of 10 to 40, 10 to 30, 10 to 20, 25 to 50, 15 to 40, 15 to 30, 20 to 50, 20 to 40, or 20 to 30 nucleotides. In some embodiments, a primer sequence has a length of 18 to 24 nucleotides.

In one embodiment, the primer sequence is located the 3′ side of the oligonucleotide used in context with the invention (i.e. the primer is in 3′ position compared to the barcode sequence).

In one embodiment, the primer sequence is selected from the group consisting of a poly-T sequence, a random DNA sequence, and a gene-specific sequence.

A “poly-T sequence” as herein referred to is a sequence comprising 10 to 50, 10 to 40, 10 to 30, 10 to 20, 25 to 50, 15 to 40, 15 to 30, 20 to 50, 20 to 40, or 20 to 30 thymine “T”. The Poly T sequence hybridizes with the poly A tail present in mRNAs.

In one embodiment, the random DNA sequence can be of any suitable length, such as 6 to 50, 6 to 50, 6 to 40, 6 to 30, 6 to 20, 10 to 50, 10 to 40, 25 to 50, 15 to 40, 15 to 30, 20 to 50, 20 to 40, or 20 to 30 nucleotides.

In one particular embodiment, the primer sequence is a gene-specific sequence and the gene is selected from the group consisting of antibody heavy chain variable gene, antibody heavy chain constant gene, antibody light chain variable gene, antibody light chain constant gene, alpha T-cell receptor gene, beta T-cell receptor gene, delta T-cell receptor gene.

The term “antibody” in the wording “antibody heavy chain variable gene”, “antibody heavy chain constant gene”, “antibody light chain variable gene” and “antibody light chain constant gene” is as defined herein above.

The term “T-cell receptor” in the wording “alpha T-cell receptor gene”, “beta T-cell receptor gene” and “delta T-cell receptor gene” or “gamma T-cell receptor gene” is as defined herein above.

The word “gene” is as defined herein above.

In one specific embodiment, the at least one type of an oligonucleotide is at least two types of oligonucleotides, wherein at least one type of oligonucleotide comprises a primer sequence specific for antibody heavy chain variable gene and wherein the other at least one type of oligonucleotide comprises a primer sequence specific for antibody light chain variable gene.

In another particular embodiment, the at least one type of an oligonucleotide is at least two types of oligonucleotides, wherein at least one type of oligonucleotide comprises a primer sequence specific for the alpha T-cell receptor gene or the beta T-cell receptor gene or gamma T-cell receptor, and wherein the other at least one type of oligonucleotide comprises a primer sequence specific for the delta T-cell receptor gene.

In another particular embodiment, the at least one type of an oligonucleotide is at least three types of oligonucleotide, wherein at least one type of oligonucleotide comprises a primer sequence specific for the alpha T-cell receptor gene and a second type of oligonucleotide comprises a primer sequence specific for the beta T-cell receptor gene, and wherein a third type of oligonucleotide comprises a primer sequence specific for the delta T-cell receptor gene.

In one embodiment, the oligonucleotide further comprises a promoter and/or a spacer sequence.

Examples of promoter sequences include, but are not limited to, T7 promoters, T3 promoters, or SP6 promoters.

It will be understood by the skilled in the art that binding the oligonucleotides temporally to a particle permits to provide particles having a high amount of oligonucleotides. Furthermore, binding the at least one type of oligonucleotide initially to the particle facilitates the introduction of oligonucleotides into each compartment, in particular into each droplet, wherein the at least one type of oligonucleotides have the same barcode sequence.

Accordingly, in one embodiment, the at least one type of oligonucleotide is covalently bonded or non-covalently bonded to the particle.

“Non-covalently bonded” herein refers, for example, to a streptavidin-biotin bond.

Other non-covalent bonds are known to the skilled in the art, such as avidin biotin bonds or his tag and nickel bonds.

“Covalently bonded” herein refers for example to an amino bond or an acrydite phosphoramidite bond.

“Streptavidin” generally refers to a 52.8 kDa protein purified from the bacterium Streptomyces avidinii. Streptavidin homo-tetramers have an extraordinarily high affinity for biotin with a dissociation constant (Kd) on the order of ≈10⁻¹⁴ mol/L, the binding of biotin to streptavidin is one of the strongest non-covalent interactions known in nature.

In a preferred embodiment, the non-covalent bond is a streptavidin-biotin bond.

Streptavidin-Biotin bonds are known to the skilled in the art. Accordingly, in one embodiment the particle as herein defined comprises streptavidin. Accordingly, in the same embodiment, the at least one type of oligonucleotide, as herein defined comprises biotin. In other words, the at least one type of oligonucleotide is functionalized with biotin.

Independent of the type of bond used to link the at least one type of oligonucleotide to the particle, the at least one type of oligonucleotide may further comprise at least one linker sequence.

Accordingly, in a further embodiment, the “at least one type of an oligonucleotide” or simply the “oligonucleotide” further comprises at least one linker sequence, said linker sequence is preferably comprised at the 5′ end. Accordingly, in one embodiment, the at least one type of an oligonucleotide comprises from 5′ to 3′ a linker sequence, a barcode sequence and a primer sequence.

In one embodiment, the “linker sequence” is a sequence with which one the oligonucleotide is optionally bonded to the particle.

“Optionally bonded herein” refers to the possibility that once the at least one type of oligonucleotide bonded to the particle is loaded into the compartment or the plurality of compartments, the at least one type of oligonucleotide might be released, so that the compartment comprises the particle and the at least one type of oligonucleotide without the at least one type of oligonucleotide being bond to said particle.

Preferably, the linker sequence is a cleavable linker sequence, e.g., that can be cleaved upon application of a suitable stimulus, such as enzymatic and/or photocleavage.

“Cleavable linkers” may include, but are not limited to, TEV, trypsin, thrombin, cathepsin B, cathespin D, cathepsin K, caspase lumatrix metalloproteinase sequences, phosphodiester, phospholipid, ester, -galactose, dialkyl dialkoxysilane, cyanoethyl group, sulfone, ethylene glycolyl disuccinate, 2-N-acyl nitrobenzenesulfonamide, a-thiophenylester, unsaturated vinyl sulfide, sulfonamide after activation, malondialdehyde (MDA)-indole derivative, levulinoyl ester, hydrazone, acylhydrazone, alkyl thioester, disulfide bridges, azo compounds, 2-Nitrobenzyl derivatives, phenacyl ester, 8-quinolinyl benzenesulfonate, coumarin, phosphotriester, bis-arylhydrazone, bimane bi-thiopropionic acid derivative, paramethoxybenzyl derivative, tert-butylcarbamate analogue, dialkyl or diaryl dialkoxysilane, orthoester, acetal, aconityl, hydrazone, b thiopropionate, phosphoramidate, imine, trityl, vinyl ether, polyketal, alkyl 2-(diphenylphosphino)benzoate derivatives, allyl ester, 8-hydroxyquinoline ester, picolinate ester, vicinal diols, and selenium compounds (see, e.g. Leriche G, Chisholm L, Wagner A.

Cleavable Linkers are well known to the skilled in the art and are further described in Chemical Biology, for example in Leriche H. et al. (Bioorg Med Chern. 15; 20(2):571-82. 2012). Cleavage conditions and reagents include, but are not limited to, enzymes, nucleophilic/basic reagents, reducing agents, photo-irradiation, electrophilic/acidic reagents, organometallic and metal reagents, and oxidizing reagents.

As mentioned above, in one embodiment, the method of the invention further comprises a step of releasing the at least some of the oligonucleotides bound to a particle from said particle prior or after lysing the cells.

The step of releasing at least some of the oligonucleotides may further occur after lysing the cells and before reverse transcribing the released nucleic acids hybridized to said oligonucleotide or after lysing the cells and after reverse transcribing the released nucleic acids hybridized to said oligonucleotide.

The skilled in the art will understand that depending on the time point selected for releasing the at least some of the oligonucleotides, the term “at least some of the oligonucleotides” might refer to, for example, at least some of the oligonucleotides hybridized to the nucleic acids released by the cells or a DNA/RNA duplex, as defined above.

In one embodiment, the at least some of the oligonucleotides can be released using any means, such as enzymes, nucleophilic/basic reagents, reducing agents, photo-irradiation, electrophilic/acidic reagents, organometallic and metal reagents, and oxidizing reagents.

In one embodiment, the at least some of the oligonucleotides can be released using enzymatic and/or photo cleavage. For example, an endonuclease may be used to cleave a linker sequence or any other sequence to release the at least some of the oligonucleotides from the particle.

In a further embodiment, releasing the oligonucleotide refers to disrupting the bond, such as a streptavidin biotin. Methods to disrupt a streptavidin biotin bond are known to the skilled in the art and include enzymatic digestion of streptavidin and/or denaturation of streptavidin.

In one embodiment, the oligonucleotide is released by enzymatic digestion of streptavidin.

The “cell” in context of the present invention is given its ordinary meaning as used in biology, for example a cell refers to an autonomous self-replicating unit that may exist as functional independent unit of life, for example for unicellular organism, or as sub-unit in a multicellular organism, for example in plants and mammals, that is specialized into carrying out particular functions towards the cause of the organism as a whole. However a “cell” may further refer to quiescent cells which typically is still capable of cell division when mitotic stimulation is applied.

In one embodiment, cells refer to prokaryotic cells or eukaryotic cells, preferably, eukaryotic cells.

The defining feature distinguishing an “eukaryotic cell” from a prokaryotic cell is that they have membrane-bound organelles, especially the nucleus, which contains the genetic material, and is enclosed by the nuclear envelope.

An “eukarvotic cell” in context of the present invention is selected from the group consisting of a mammal cells, plant cell and fungal cell, preferably mammalian cell.

A “mammal” herein refers to any mammal, including humans, domestic and farm animals, and zoo, sports, or pet animals, such as dogs, cats, cattle, horses, sheep, pigs, goats, rabbits, etc. Preferably, the mammal is human.

Accordingly, in one embodiment, the cell is a mammalian cell, an engineered mammalian cell or a cell line or a mammalian immune cell.

In one embodiment, the mammalian cell is an immune cell.

In one embodiment, an immune cell may be, but is not limited to, B cells, T cells, or hybridomas, preferably B cell.

In one embodiment, a cell or plurality of cells herein refers to different types of cells or to cells of the same type or origin exposed to different conditions.

In one particular embodiment, the cell is a non-mammalian cell.

In one further particular embodiment, the non-mammalian cell is a yeast cell, an avian cell or a shark cell.

Step d) of the methods of the invention refers to reverse transcribing the released nucleic acids hybridized to said oligonucleotide using the primer sequence in at least some of the compartments. Reverse transcription is performed using the reverse transcriptase (RT) comprised in at least some of the compartment.

The “reverse transcriptase (RT)” in context of the present invention is an enzyme used to generate complementary DNA (cDNA) from an RNA template, in a process termed reverse transcription.

In one embodiment, the reverse transcriptase is selected from the group consisting of Superscriptase I, Superscriptase II, Superscriptase Ill, Superscriptase IV, Murine Leukemia RT, SmartScribe RT or MultiScribe RT.

In one embodiment, the reverse transcriptase is at a concentration of 1 to 50 U/μl, preferably 5 to 25 U/μl, for example at 12.5 U/μl.

“Reverse Transcription” or “RT reaction” is a process in which single-stranded RNA is reverse transcribed into a single-stranded complementary DNA (cDNA) by using total cellular RNA or poly(A) RNA, a reverse transcriptase enzyme, a primer, dNTPs and an RNase inhibitor. It will be understood by the skilled in the art, that the product of the reverse transcription is a RNA/DNA duplex comprising a single strand cDNA hybridized to its template RNA. As it will be further understood, said RNA/DNA duplex is further linked to the oligonucleotide comprising the primer sequence used for the reverse transcription.

Accordingly, it will be understood by the skilled in the art, that after reverse transcribing the nucleic acids in step d) of the methods of the invention the compartment or the plurality of compartments further comprises cDNAs.

Accordingly, in one embodiment, the at least some of the compartments further comprise single cell cDNAs produced by reverse transcription of nucleic acids from the single cell lysate.

In one embodiment, said cDNA refers to a single-stranded complementary DNA.

In a further embodiment, said cDNA is comprised in a RNA/DNA duplex.

In one embodiment, the RNA/DNA duplex refers to the RNA that has been reverse transcribed and is hybridized to the primer sequence of the at least one type of oligonucleotide.

As it will be understood by the skilled in the art, in one embodiment, the RNA/DNA duplex is linked to the oligonucleotide comprising the primer sequence to which the nucleic acid, preferably mRNA was hybridized in step c) and which was used for reverse transcription in step d).

In one embodiment, the compartment or plurality of compartments comprise a reverse transcriptase composition.

In one embodiment, a reverse transcriptase composition comprises a protease inhibitor, dNTPs and/or DTT, preferably protease inhibitor, dNTPs and DTT.

In one embodiment, DTT is at a concentration of 1 mM to 10 mM, preferably 5 mM.

In one embodiment, the protease inhibitor comprises a plurality of protease inhibitors.

In one embodiment, the protease inhibitor is selected from the list consisting of Leupeptin hemisulfate salt, pepstatin A, AEBSF, Aprotinin, Bestatin hydrochloride, E-64 and PMSF.

For example, the protease inhibitor may comprise one or more of Leupeptin hemisulfate salt, pepstatin A, AEBSF, Aprotinin, Bestatin hydrochloride, E-64 and PMSF.

As used herein, the term “dNTP” refers to a deoxynucleoside triphosphate, e.g. deoxyadenosine-5′-triphosphate (dATP, “A”), deoxycytidine-5′-triphosphate (dCTP, “C”), deoxyguanosine-5′-triphosphate (dGTP, “G”), deoxythymidine-5′-triphosphate (dTTP, “T”) or deoxyuridine-5′-triphosphate (dUTP, “U”). The term “dNTP” is intended to refer also to deoxynucleoside triphosphates comprising modified bases and base analogues that are capable of mimicking the base pairing of A, C, G, T, or U, or that are capable of base pairing in a degenerate mode, e.g., a base that pairs with A or G, C or T, A or C, G or T, G or C, or A or T, called nucleotide analogues. Said nucleotide analogues may be used, for example, for purification, as further explained herein below.

In one embodiment, the dNTP is at a concentration of 0.01 mM to 10 mM, preferably 0.1 to 1 mM, more preferably 0.5 mM.

In one embodiment, a reverse transcriptase composition further comprises a RNase inhibitor.

In context of the present invention, the compartment or plurality of compartments comprises an aqueous composition.

The “aqueous composition” in context of the invention is typically adapted to the cells used in the method of the invention and typically comprises a buffered solution as defined herein below.

Step b) of the method of the invention refers to lysing at least some of the cells within the compartments.

Said “cell lysis” in context of the present invention may be accomplished by enzymatic, physical, and/or chemical means, or any combination thereof, in particular enzymatic, physical, and/or chemical means. Other cell disruption methods may be also be used.

Accordingly, in one embodiment, the compartment or plurality of compartments are lysed in step a) using enzymatic, physical, and/or chemical cell lysis.

“Enzymatic methods” to remove cell walls is well-established in the art. The enzymes are generally commercially available and, in most cases, were originally isolated from biological sources. Enzymes commonly used include lysozyme, lysostaphin, zymolase, mutanolysin, glycanases, proteases, and mannose.

As known by the skilled in the art “chemical cell lysis” is achieved using chemicals such as detergents, which disrupt the lipid barrier surrounding cells by disrupting lipid-lipid, lipid-protein and protein-protein interactions. The ideal detergent for cell lysis depends on cell type and source. Nonionic and zwitterionic detergents are milder detergents. The Triton X series of nonionic detergents and 3-[(3-Cholamidopropyl)dimethylammonio]-1-propanesulfonate (CHAPS), a zwitterionic detergent, are commonly used for these purposes. In contrast, ionic detergents are strong solubilizing agents and tend to denature proteins, thereby destroying protein activity and function. SDS, an ionic detergent that binds to and denatures proteins, is used extensively in the art to disrupt cells.

“Physical cell lysis” refers to the use of sonication, ice shock or electroporation.

In one example the cells within the compartments are lysed on ice.

In one preferred embodiment, the cell lysis of step a) does not destroy the compartments, in particular the droplets, in context of the invention.

According to the above, in one embodiment, the compartment or plurality of compartments comprise a lysis composition.

In one embodiment, the lysis composition comprises enzymes selected from the group consisting of lysozyme, lysostaphin, zymolase, mutanolysin, glycanases, proteases, and mannose.

In one preferred embodiment, the lysing composition in context of the present invention comprises magnesium chloride, a detergent, a buffered solution and an RNase inhibitor.

In one embodiment, the magnesium chloride is used at a concentration of between 1 mM to 20 mM.

In one embodiment, the detergent is selected from the group consisting of Triton-X-100, NP-40, Nonidet P40, and Tween-20 and IGEPAL CA 630.

In one embodiment, the detergent is at a concentration of 0.1% to 10%.

Nonlimiting examples of the buffered solution may include Tris-HCl, Hepes-KOH, Pipes-NaOH, maleic acid, phosphoric acid, citric acid, malic acid, formic acid, lactic acid, succinic acid, acetic acid, pivalic (trimethylacetic) acid, pyridine, piperazine, picolinic acid, L-histidine, MES, Bis-tris, bis-tris propane, ADA, ACES, MOPSO, PIPES, imidazole, MOPS, BES, TES, HEPES, DIPSO, TAPSO, TEA (triethanolamine), N-Ethylmorpholine, POPSO, EPPS, HEPPS, HEPPSO, Tris, tricine, Glycylglycine, bicine, TAPS, morpholine, N-Methyldiethanolamine, AMPD (2-amino-2-methyl-1,3-propanediol), Diethanolamine, AMPSO, boric acid, CHES, glycine, CAPSO, ethanolamine, AMP (2-amino-2-methyl-1-propanol), piperazine, CAPS, 1, 3-Diaminopropane, CABS, or piperidine (see also, www.reachdevices.com/Protein/BiologicalBuffers.html). Nonlimiting examples of RNase inhibitors may include RNase OUT, IN, SuperlN Rnase, and those inhibitors targeting a wide range of RNAse (e.g., A, B, C, 1 and T1).

In one example the lysis composition is typically 0.2% Triton, 3 mM MgCl₂, 50 mM Tris-HCl pH 7.4.

Step c) of the method of the invention refers to hybridizing at least some of the released nucleic acids to said oligonucleotide in at least some of the compartments.

The hybridization of step c) herein refers to a phenomenon in which the primer sequence present in the oligonucleotide anneals to a complementary nucleic acid sequence of the released nucleic acids, accordingly, as known by the skilled in the art, the temperature to use depends on the primer sequence and/or the RT enzyme used.

In one example, step c) and d) are performed by incubating the compartments for example for 1 h at 55° C. or 2 h at 50° C. during typically mixing of the compartments at for example 550 rpm.

In one embodiment, the cDNAs produced by the reverse transcription of step d) are recovered and further used for, typically, subsequent amplification and sequencing library preparation.

Accordingly, in one embodiment, the method of the invention further comprises recovering single cell cDNAs produced by reverse transcription in at least some of the compartments.

“Recovering” herein refers to isolating the cDNAs produced by reverse transcription in at least some of the compartments from said plurality of compartments.

In one embodiment, recovering herein refers to collecting the compartments comprising cDNA produced by reverse transcription or collecting the aqueous composition contained in said compartments comprising said cDNA, and separating the cDNA comprised in the aqueous composition.

In one particular embodiment, recovering herein refers to collecting the microfluidic droplets comprising cDNA produced by reverse transcription, breaking the microfluidic droplets and separating the cDNA comprised in the aqueous composition from the oil phase of said microfluidic droplets.

Methods to isolate nucleic acids, in particular cDNA from microfluidic droplets are known to the skilled in the art and comprise for example, collecting the microfluidic droplets and breaking the microfluidic droplets using typically perfluoro-octanol (v/v emulsion). Then incubating the emulsion obtained in the previous step until the aqueous and oil phase are separated. In one example, the aqueous phase is typically centrifuged for, for example, 10 min at 10000 g at 4° C. and the supernatant comprising the cDNA is recovered.

In one embodiment, the method further comprises the step of removing unincorporated oligonucleotides, preferably, removing unincorporated oligonucleotides from the aqueous composition of the compartments. In one preferred embodiment, the step of removing unincorporated oligonucleotides composition from the at least some of the compartments takes place after the step of recovering the cDNA produced by reverse transcription as defined herein above.

Preferably, the step of removing unincorporated oligonucleotides precedes the amplification step and/or the sequencing step defined herein below.

It will be understood by the skilled in the art that the step of removing unincorporated oligonucleotides encompasses removing unincorporated barcode sequences.

In one embodiment, removing unincorporated oligonucleotides comprises contacting the aqueous composition of the at least some of the compartments with a purification substrate wherein the purification substrate removes unincorporated oligonucleotides. In one embodiment, the purification substrate comprises beads or particles, which, optionally, form a column. In a further example, unincorporated oligonucleotides are removed by size selection using for example an acrylamide gel.

In one embodiment, the step of removing unincorporated oligonucleotides comprises contacting the aqueous composition of the at least some of the compartments with an exonuclease to degrade the unincorporated oligonucleotides within the aqueous composition of the at least some of the compartments.

In certain embodiments of this step, the exonuclease degrades single stranded nucleic acid sequences from the aqueous compositions comprising the cDNA.

It will be understood by the skilled in the art, that the cDNA obtained in step d) is typically present in form of a RNA/DNA complex and thus protected from said exonucleases.

In one embodiment, the cDNA comprises one or more nucleotide analogs, as defined herein above, facilitating purification of the cDNA sequences or molecules.

As it will be understood by the skilled in the art, in certain embodiments, purified cDNA does not comprise unincorporated oligonucleotides. Accordingly, in certain embodiments, purified cDNA does not comprise unincorporated barcode sequences.

In one embodiment, the cDNA is further treated with RNAse A and/or RNAse H.

“RNAse A” is an endoribonuclease that specifically degrades single-stranded RNA at C and U residues.

In one embodiment, the RNAse A is at a concentration of 10 to 1000 μg/μL, preferably 50 to 200 μg/μL, for example at 100 μg/μL.

“RNAse H” is a family of non-sequence-specific endonucleases that catalyze the cleavage of RNA via a hydrolytic mechanism. RNase H's ribonuclease activity cleaves the 3′-O—P bond of RNA in a DNA/RNA duplex substrate to produce 3′-hydroxyl and 5′-phosphate terminated products.

In one embodiment, the RNAse H is at a concentration of 10 to 1000 μg/μL, preferably 50 to 200 μg/μL, for example at 100 μg/μL.

In one embodiment, the cDNA is further treated with Proteinase K.

“Proteinase K” is a broad-spectrum serine protease and digests proteins, preferentially after hydrophobic amino acids.

In one embodiment, the Proteinase K is at a concentration of 0.1 to 5 mg/mL, preferably 0.1 to 1 mg/mL, for example at 0.8 mg/mL.

In one embodiment the method further comprises the step of amplifying the cDNA obtained in step d) of the method of the invention. In one embodiment, said amplification step is performed after removing unincorporated oligonucleotides. In one embodiment, said amplification step is performed prior to the sequencing step defined herein below.

In one embodiment, the amplifying step is performed in a multiplex reaction, a separated polymerase chain reaction (PCR), or a linear amplification.

In one embodiment, the linear amplification is an in vitro transcription.

In one embodiment, the cDNA produced in step d) is quantified using qPCR, such as simplex and/or multiplex qPCR reaction.

In a further embodiment the method further comprises a step of sequencing the cDNA obtained in step d).

In context of the present invention, in one embodiment, the step of sequencing the cDNA herein refers to first contacting the cDNA to a sequencing library and amplifying the sequences of interest from the sequencing library that correspond to the cDNA, respectively.

In one embodiment, the step of sequencing the cDNA may comprise performing a next generation sequencing (NGS) protocol on a sequencing library.

In certain embodiments, the NGS protocol comprises loading an amount of the sequencing library between 4 μM to 20 μM per flow cell of a reagent kit.

In one embodiment, the NGS sequencing protocol further comprises the step of adding 5-60% PhiX to the amount of the sequencing library or to the flow cell of the reagent kit.

### Plurality of Microfluidic Droplets

The invention further refers to a plurality of compartments wherein at least some of the compartments comprise (i) a single cell or a single cell lysate comprising nucleic acids and (ii) at least one type of oligonucleotide, and (iii) a reverse transcriptase, wherein the at least one type of oligonucleotide comprises a barcode sequence and a primer sequence, wherein each different primer sequence defines a different oligonucleotide type, and wherein the compartments of the plurality of the compartments contain one or more barcode sequences distinguishable from barcode sequences contained in other compartments of the plurality of compartments;
wherein the plurality of compartments has a volume of less than 3 nL; and
preferably wherein the concentration of each type of oligonucleotide in the compartments is at least 100 nM.

As explained herein above, in some embodiments, the at least one type of oligonucleotide is introduced into the compartments by initially binding the at least one oligonucleotide to a particle (e.g., a hydrogel or a polymeric particle or magnetic particle).

Accordingly, in one embodiment, the at least some of the compartments further comprise (iv) a particle, wherein the at least one type of oligonucleotide of ii) is preferably bonded to the particle of iv).

Accordingly, in one particular embodiment the invention further refers to a plurality of compartments wherein at least some of the compartments comprise (i) a single cell or a single cell lysate comprising nucleic acids and (ii) at least one type of oligonucleotide, (iii) a reverse transcriptase and (iv) a particle, wherein the at least one type of oligonucleotide of ii) is optionally bonded to said particle (iv), and wherein the at least one type of oligonucleotide comprises a barcode sequence and a primer sequence, wherein each different primer sequence defines a different oligonucleotide type, and wherein the compartments of the plurality of the compartments contain one or more barcode sequences distinguishable from barcode sequences contained in other compartments of the plurality of compartments;
wherein the plurality of compartments has a volume of less than 3 nL; and
preferably wherein the concentration of each type of oligonucleotide in the compartments is at least 100 nM.

In one embodiment, the at least one type of oligonucleotide of iii) might be non-covalently bonded or covalently bonded to the particle of iv). Non-covalent bonds and covalent bonds are as defined herein above.

In one particular embodiment, the plurality of compartments has a volume equal to or less than 1 nL.

In one embodiment, at least some of the compartments further comprise v) single cell cDNAs produced by reverse transcription of nucleic acids from the single cell lysate.

The terms “cell”, “nucleic acids”, “oligonucleotide” or “each type of oligonucleotide”, “reverse transcriptase”, “barcode sequence”, “primer sequence”, “volume of compartments”, “concentration of each type of oligonucleotide”, “particle”, “bonded” are as defined herein above in the section “Method for capturing and barcoding single cell nucleic acid”.

The features described in the previous section “Method for capturing and barcoding single cell nucleic acid” are entirely applicable to the instant description of the plurality of compartments.

### A Method for Preparing a Plurality of Microfluidic Droplets

The invention further refers to a method for preparing a plurality of microfluidic droplets comprising the following steps:
providing a first fluid source, the first fluid comprising a suspension of cells,
providing a second fluid source, the second fluid comprising at least one type of an oligonucleotide, wherein the at least one type of oligonucleotide comprises one barcode sequence and a primer sequence, wherein each different primer sequence defines a different oligonucleotide type, and wherein the droplets of the plurality of the microfluidic droplets contain one or more barcode sequences distinguishable from barcode sequences contained in other droplets of the plurality of microfluidic droplets;
providing a carrier fluid, the carrier fluid being immiscible with the first fluid and the second fluid,
injecting the carrier fluid in a main channel of a chip,
generating a flow of droplets in the carrier fluid by injecting the second fluid and the first fluid in at least a secondary channel of the chip, the secondary channel opening in the main channel, each generated droplet comprising a mix of the first fluid and the second fluid,
wherein the concentration of the cells in the first fluid, the concentration of oligonucleotides in the second fluid, the geometry of the main channel and the secondary channel, the injection parameters of the first fluid, of the second fluid and of the carrier fluid are adapted such that each droplet comprises only a single cell and presents a volume of less than 3 nL, and, preferably, such that the concentration of each type of oligonucleotide in each droplet is at least 100 nM.

In one embodiment, the second fluid further comprises a plurality of particles, each particle comprising at least one type of an oligonucleotide bonded thereto, wherein the term “bonded” is as defined herein above.

In the same embodiment, the concentration of the cells in the first fluid, the concentration of particles in the second fluid, the number of oligonucleotides bonded to each particle, the geometry of the main channel and the secondary channel, the injection parameters of the first fluid, of the second fluid and of the carrier fluid are adapted such that each droplet comprises only a single cell and a particle and presents a volume of less than 3 nL, and, preferably, such that the concentration of each type of oligonucleotide in each droplet is at least 100 nM.

In one embodiment, the second fluid further comprises the reverse transcriptase, wherein the reverse transcriptase is as defined herein above.

In one embodiment, the second fluid further comprises a lysis composition.

In one embodiment, the second fluid further comprises the reverse transcription composition as defined herein above.

In an alternative embodiment, the second fluid does not comprise the reverse transcription composition and does not comprise the reverse transcriptase, in the same embodiment, the method for preparing a plurality of microfluidic droplets further comprises providing a third fluid source, the third fluid comprising the reverse transcriptase.

In a related embodiment, the third fluid further comprises a reverse transcriptase composition.

Accordingly, in a further embodiment the invention refers to a method for preparing a plurality of microfluidic droplets comprising the following steps:
providing a first fluid source, the first fluid comprising a suspension of cells,
providing a second fluid source, the second fluid comprising at least one type of an oligonucleotide, wherein the at least one type of oligonucleotide comprises one barcode sequence and a primer sequence, wherein each different primer sequence defines a different oligonucleotide type, and wherein the droplets of the plurality of the microfluidic droplets contain one or more barcode sequences distinguishable from barcode sequences contained in other droplets of the plurality of microfluidic droplets;
providing a third fluid source, the third fluid comprising the reverse transcriptase,
providing a carrier fluid, the carrier fluid being immiscible with the first fluid, the second fluid and the third fluid,
injecting the carrier fluid in a main channel of a chip,
generating a flow of droplets in the carrier fluid by injecting the first fluid, the second fluid and the third fluid in at least a secondary channel of the chip, the secondary channel opening in the main channel, each generated droplet comprising a mix of the first fluid, the second and the third fluid,
wherein the concentration of the cells in the first fluid, the concentration of oligonucleotides in the second fluid, the geometry of the main channel and the secondary channel, the injection parameters of the first fluid, the second fluid, the third fluid and of the carrier fluid are adapted such that each droplet comprises only a single cell and a particle and presents a volume of less than 3 nL, and, preferably, such that the concentration of each type of oligonucleotide in each droplet is at least 100 nM.

In the same embodiment, the second fluid may further comprise a plurality of particles; each particle comprising at least one type of an oligonucleotide bonded thereto, wherein the term “bonded” is as defined herein above.

In the same embodiment, the concentration of the cells in the first fluid, the concentration of particles in the second fluid, the number of oligonucleotides bonded to each particle, the geometry of the main channel and the secondary channel, the injection parameters of the first fluid, the second fluid and the third fluid and of the carrier fluid are adapted such that each droplet comprises only a single cell and a particle and presents a volume of less than 3 nL, and, preferably, such that the concentration of each type of oligonucleotide in each droplet is at least 100 nM.

In a related embodiment, the second fluid or the third fluid may further comprise a lysis composition.

In a further related embodiment, the third fluid further comprises a reverse transcriptase composition.

In a further embodiment, the invention refers to a method for preparing a plurality of microfluidic droplets comprising the following steps:
providing a first fluid source, the first fluid comprising a suspension of cells,
providing a second fluid source, the second fluid comprising at least one type of an oligonucleotide, wherein the at least one type of oligonucleotide comprises one barcode sequence and a primer sequence, wherein each different primer sequence defines a different oligonucleotide type, and wherein the droplets of the plurality of the microfluidic droplets contain one or more barcode sequences distinguishable from barcode sequences contained in other droplets of the plurality of microfluidic droplets;
providing a third fluid source, the third fluid comprising the reverse transcriptase,
providing a fourth fluid source, the fourth fluid comprising the lysis composition,
providing a carrier fluid, the carrier fluid being immiscible with the first fluid, the second fluid, the third fluid and the fourth fluid,
injecting the carrier fluid in a main channel of a chip,
generating a flow of droplets in the carrier fluid by injecting the first fluid, the second fluid, the third fluid and the fourth fluid in at least a secondary channel of the chip, the secondary channel opening in the main channel, each generated droplet comprising a mix of the first fluid, the second, the third fluid and the fourth fluid,
wherein the concentration of the cells in the first fluid, the concentration of oligonucleotides, the geometry of the main channel and the secondary channel, the injection parameters of the first fluid, the second fluid, the third fluid, the fourth fluid and of the carrier fluid are adapted such that each droplet comprises only a single cell and a particle and presents a volume of less than 3 nL, and, preferably, such that the concentration of each type of oligonucleotide in each droplet is at least 100 nM.

In a related embodiment, the third fluid further comprises a reverse transcriptase composition.

In one embodiment, the second fluid further comprises a plurality of particles; each particle comprising at least one type of an oligonucleotide bonded thereto, wherein the term “bonded” is as defined herein above.

In the same embodiment, the concentration of the cells in the first fluid, the concentration of particles in the second fluid, the number of oligonucleotides bonded to each particle, the geometry of the main channel and the secondary channel, the injection parameters of the first fluid, the second fluid, the third fluid, the fourth fluid and of the carrier fluid are adapted such that each droplet comprises only a single cell and a particle and presents a volume of less than 3 nL, and, preferably, such that the concentration of each type of oligonucleotide in each droplet is at least 100 nM.

It will be understood that in the methods described herein above, in one embodiment, the particles and the cells may be encapsulated within the droplets simultaneously or sequentially, in any suitable order.

The “lysis composition” comprises the same ingredients as defined herein above in the section “Method for capturing and barcoding single cell nucleic acid”. However, as it will be understood by the skilled in the art, the concentration of the ingredients present in the lysis composition of the second fluid will be increased in comparison to the concentration of the ingredients present in the plurality of microfluidic droplets that are obtained by set method. In other words, the concentration of the ingredients of the lysis composition present in the plurality of microfluidic droplets will be decreased in comparison to the concentration of the ingredients present in the second fluid.

For example, the concentrations of the ingredients of the lysis composition present in the second fluid may be 3 to 7 times higher, preferably, 3 to 5 times higher, for example 4 times higher than the concentrations present in the final microfluidic droplet or the plurality of microfluidic droplets.

The “reverse transcriptase composition”, in context of the method for preparing a plurality of microfluidic droplets, comprises the same ingredients as defined herein above.

However, as it will be further understood by the skilled in the art, the concentration of the ingredients contained in the reverse transcriptase composition of the second or third fluid will be increased in comparison to the concentration of the ingredients contained in the reverse transcriptase composition present in the plurality of microfluidic droplets that are obtained by said method.

For example, the concentrations of the ingredients of the reverse transcriptase composition present in the second or third fluid may be 2 to 7 times higher, preferably, 2 to 5 times higher, for example 2.6 times higher than the concentrations present in the plurality of microfluidic droplets, which is as defined herein above.

In one embodiment, the first and second fluid sources are organized in the form of a junction.

In a further embodiment, the first, second and third fluid sources are organized in the form of a junction.

In a further embodiment, the first, second, third fluid and fourth fluid sources are organized in the form of a junction.

The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et ah, published as WO 2004/091763 on Oct. 28, 2004, or International Patent Application No. PCT/US2003/020542, filed Jun. 30, 2003, entitled “Method and Apparatus for Fluid Dispersion,” by Stone, et ah, published as WO 2004/002627 on Jan. 8, 2004.

In some embodiments, the junction may be configured and arranged to produce substantially monodisperse droplets.

In one embodiment, the plurality of microfluidic droplets are prepared in such a way that there is no more than about 2 particle/droplet, preferably, the plurality of microfluidic droplets are prepared in such a way that there is no more than about 1 particle/droplet or microfluidic droplets are prepared preferentially with 1 particle/droplet, or the microfluidic droplets are prepared with an average of 1 particle/droplet.

The amount of particles/droplet may also be referred to as loading rate. For example, the average loading rate may be less than about 1 particle/droplet, less than about 0.9 particles/droplet, less than about 0.8 particles/droplet, less than about 0.7 particles/droplet, less than about 0.6 particles/droplet, less than about 0.5 particles/droplet, less than about 0.4 particles/droplet, less than about 0.3 particles/droplet, less than about 0.2 particles/droplet, less than about 0.1 particles/droplet, less than about 0.05 particles/droplet, less than about 0.03 particles/droplet, less than about 0.02 particles/droplet, or less than about 0.01 particles/droplet. In some cases, lower particle loading rates may be chosen to minimize the probability that a droplet will be produced having two or more particles in it. Thus, for example, at least about 50%, at least about 60%, at least about 70%, at least about 80%, at least about 90%, at least about 95%, at least about 97%, at least about 98%, or at least about 99% of the droplets may contain either no particle or only one particle.

Similarly, in some embodiments, the plurality of microfluidic droplets are prepared in such a way that, on the average, each droplet has less than 1 cell in it. In the same embodiments the amounts of cells/droplet may be referred to as loading rate.

For example, the average loading rate may be less than about 1 cell/droplet, less than about 0.9 cells/droplet, less than about 0.8 cells/droplet, less than about 0.7 cells/droplet, less than about 0.6 cells/droplet, less than about 0.5 cells/droplet, less than about 0.4 cells/droplet, less than about 0.3 cells/droplet, less than about 0.2 cells/droplet, less than about 0.1 cells/droplet, less than about 0.05 cells/droplet, less than about 0.03 cells/droplet, less than about 0.02 cells/droplet, or less than about 0.01 cells/droplet. In some cases, lower cell loading rates may be chosen to minimize the probability that a droplet will be produced having two or more cells in it. Thus, for example, at least about 50%, at least about 60%, at least about 70%, at least about 80%, at least about 90%, at least about 95%, at least about 97%, at least about 98%, or at least about 99% of the droplets may contain either no cell or only one cell. In addition, it should be noted that the average rate of particle loading and the average rate of cell loading within the droplets may the same or different.

In some cases, a relatively large number of droplets may be prepared per second, e.g., at least about 10 per second, at least about 30 per second, at least about 50 per second, at least about 100 per second, at least about 300 per second, at least about 500 per second, at least about 1,000 per second, at least about 3,000 per second, at least about 5,000 per second, at least about 10,000 per second, at least about 30,000 per second, at least about 50,000 per second, at least about 100,000 droplets per second, etc. In particular, 1 to 2000 droplets may be prepared per second, such as 1 to 800 droplets per second, 1 to 700 droplets per second, 1 to 600 droplets per second, 1 to 500 droplets per second, 1 to 400 droplets per second, 1 to 300 droplets per second, 1 to 200 droplets per second, 1 to 100 droplets per second, 1 to 80 droplets per second, 1 to 70 droplets per second, 1 to 50 droplets per second, for example 10 to 300 microfluidic droplets per second, 50 to 300 droplets per second, 100 to 300 droplets per second, 150 to 300 droplets per second, 150 to 250 droplets per second, 175 to 250 microfluidic droplets per second, typically, 1 to 1000 droplets per second, preferably 175 to 250 microfluidic droplets per second.

In some cases, as previously discussed, some or all of the droplets may be distinguishable, e.g., on the basis of the type of oligonucleotides present in at least some of the droplets (e.g., which preferably comprises one barcode sequence as defined herein above). In some cases, at least about 50%, at least about 60%, at least about 70%, at least about.

The volume of the plurality of droplets is as defined herein above in the section “Method for capturing and barcoding single cell nucleic acid”.

In one embodiment, after preparing a plurality of microfluidic droplets according to the method herein above, the method may further comprise the steps b) to d) of the Method for capturing and barcoding single cell nucleic acid as defined herein above and other further steps of the Method for capturing and barcoding single cell nucleic acid as defined herein above.

The invention further relates to items as described in the priority application U.S. 62/264,414, the content of which is incorporated herein by reference.

Accordingly, the invention further refers to a method for identifying at least two gene sequences from at least one cell comprising,
(a) encapsulating at least a first cell, a first carrier linked to a plurality of a first unique DNA barcode, and a reverse transcriptase enzyme to form a first aqueous composition, under conditions such that the first cell releases at least two RNA molecules within the first aqueous composition;
   under conditions such that the first unique DNA barcode hybridizes to each of the at least two RNA molecules to form at least two DNA/RNA duplexes, and under conditions such that at least two cDNA transcripts comprising the first unique DNA barcode are generated within the first aqueous composition,
   wherein the volume of the first aqueous composition is less than 5 nanoliters;
(b) removing unincorporated DNA barcode sequences; and
(c) sequencing each of the at least two cDNA transcripts;
   thereby identifying at least two gene sequences from at least one cell.

According to a further item, the invention refers to a method for identifying at least two gene sequences from at least one cell comprising,
(a) encapsulating at least a first cell and a first carrier linked to a plurality of a first unique DNA barcode to form a first aqueous composition,
   under conditions such that the first cell releases at least two RNA molecules within the first aqueous composition; and
   under conditions such that the first unique DNA barcode hybridizes to each of the at least two RNA molecules to form at least two DNA/RNA duplexes,
(b) contacting the first aqueous composition with a reverse transcriptase enzyme under conditions such that at least two cDNA transcripts comprising the first unique DNA barcode are generated within the first aqueous composition,
   wherein the volume of the first aqueous composition is less than 5 nanoliters;
(c) removing unincorporated DNA barcode sequences; and
(d) sequencing each of the at least two cDNA transcripts;
   thereby identifying at least two gene sequences from at least one cell.

According to one item, the methods of the invention further comprise the step of amplifying each of the at least two cDNA transcripts prior to sequencing each of the at least two cDNA transcripts.

According to one item, the amplifying step is performed in a multiplex reaction, a separated polymerase chain reaction (PCR), or a linear amplification.

According to one item, the linear amplification is an in vitro transcription.

According to a further item, the methods of the invention further comprise the step of releasing each of the at least two DNA/RNA duplexes from the first carrier within the first aqueous composition prior to amplifying each of the at least two cDNA transcripts.

According to another item, the methods of the invention further comprise the step of releasing each of the at least two DNA/RNA duplexes from the first carrier within the first aqueous composition after amplifying each of the at least two cDNA transcripts.

According to one item, the methods of the invention further comprise the step of releasing each of the at least two DNA/RNA duplexes from the first carrier within the first aqueous composition prior to contacting the first aqueous composition with the reverse transcriptase enzyme.

According to a further item, the first aqueous composition further comprises a first lysing composition.

According to a further item, the first aqueous composition further comprises a first reverse transcriptase composition.

According to a further item, the first cell is an immune cell.

According to a further item, the immune cell is a B cell, a T cell, or a hybridoma, preferably a B cell.

According to another item, the first cell is a mammalian cell.

According to another item, the first cell is a non-mammalian cell.

According to one item, the non-mammalian cell is a yeast cell.

According to another item, the non-mammalian cell is an avian cell.

According to another item, the non-mammalian cell is a shark cell.

According to a further item, the at least two RNA molecules comprise a first RNA molecule comprising a first target sequence and a second RNA molecule comprising a second target sequence.

According to a particular item, the first cell is an immune cell; the at least two RNA molecules comprise a first RNA molecule comprising a sequence that encodes a heavy chain variable region (VH) and a second RNA molecule comprising a sequence that encodes a light chain variable region (VL);
the at least two DNA/RNA duplexes comprisea first DNA/RNA duplex comprising a sequence that encodes a heavy chain variable region (VH) and a second DNA/RNA duplex comprising a sequence that encodes a light chain variable region (VL); and
the at least two cDNA transcripts comprising the first unique DNA barcode comprise a first cDNA transcript comprises a sequence that encodes a VH and the first unique DNA barcode sequence and a second cDNA transcript comprises a sequence that encodes a light chain variable region (VL) and the first unique DNA barcode sequence.

According to a particular item, the first cell is an immune cell; the at least two RNA molecules comprise a first RNA molecule comprising a sequence that encodes a T-cell Receptor α-chain (TCR-α) and a second RNA molecule comprising a sequence that encodes a T-cell Receptor β-chain (TCR-β);
the at least two DNA/RNA duplexes comprise a first DNA/RNA duplex comprising a sequence that encodes a TCR-α and a second DNA/RNA duplex comprising a sequence that encodes a TCR-β; and
the at least two cDNA transcripts comprising the first unique DNA barcode comprise a first cDNA transcript comprises a sequence that encodes a TCR-α and the first unique DNA barcode sequence and a second cDNA transcript comprises a sequence that encodes TCR-β and the first unique DNA barcode sequence.

According to one item, the at least two RNA molecules comprise a first RNA molecule comprising a polyA sequence and a second RNA molecule comprising a polyA sequence.

According to a further item, the first unique DNA barcode sequence comprises a DNA sequence comprising, from 5′ to 3′, a first unique barcode sequence, wherein the barcode sequence is single- or double-stranded, and a first priming sequence for reverse-transcription, wherein the priming sequence is single-stranded.

According to a further item, the first unique DNA barcode sequence further comprises a linker sequence 5′ to the first unique barcode sequence and the first priming sequence.

According to a further item, the method further comprises,
(a) encapsulating at least a second cell, a second carrier linked to a plurality of a second unique DNA barcode, and a reverse transcriptase enzyme to form a second aqueous composition,
   under conditions such that the second cell releases at least two RNA molecules within the second aqueous composition;
   under conditions such that the second unique DNA barcode hybridizes to each of the at least two RNA molecules to form at least two DNA/RNA duplexes, and
   under conditions such that at least two cDNA transcripts comprising the second unique DNA barcode are generated within the second aqueous composition,
   wherein the volume of the second aqueous composition is less than 5 nanoliters;
(b) removing unincorporated DNA barcode sequences; and
(c) sequencing each of the at least two cDNA transcripts.

According to another item, the method further comprises,
(a) encapsulating at least a second cell and a second carrier linked to a plurality of a second unique DNA barcode to form a second aqueous composition, under conditions such that the second cell releases at least two RNA molecules within the second aqueous composition; and
   under conditions such that the second unique DNA barcode hybridizes to each of the at least two RNA molecules to form at least two DNA/RNA duplexes,
(b) contacting the second aqueous composition with a reverse transcriptase enzyme under conditions such that at least two cDNA transcripts comprising the second unique DNA barcode are generated within the second aqueous composition, wherein the volume of the second aqueous composition is less than 5 nanoliters;
(b) removing unincorporated DNA barcode sequences; and
(c) sequencing each of the at least two cDNA transcripts.

According to one item, the methods of the invention further comprise the step of amplifying each of the at least two cDNA transcripts prior to sequencing each of the at least two cDNA transcripts.

According to one item, said amplifying step is performed in a multiplex reaction, a separated polymerase chain reaction (PCR), or a linear amplification.

According to a further item, the linear amplification is an in vitro transcription.

According to another item, the methods of the invention further comprise the step of releasing each of the at least two DNA/RNA duplexes from the second carrier within the second aqueous composition prior to amplifying each of the at least two cDNA transcripts.

According to another item, the methods of the invention further comprise the step of releasing each of the at least two DNA/RNA duplexes from the second carrier within the second aqueous composition after amplifying each of the at least two cDNA transcripts.

According to further item, the methods of the invention further comprise the step of releasing each of the at least two DNA/RNA duplexes from the second carrier within the second aqueous composition prior to contacting the second aqueous composition with the reverse transcriptase enzyme.

According to one item, the second aqueous composition further comprises a second lysing composition.

According to another item, the second aqueous composition further comprises a second reverse transcriptase composition.

According to a further item, the second cell is an immune cell.

According to a further item, the immune cell is a B cell, a T cell, or a hybridoma, preferably a B cell.

According to another item, the second cell is a mammalian cell.

According to another item, the second cell is a non-mammalian cell as defined according to one of the items herein above.

According to one particular item, the second cell is an immune cell; the at least two RNA molecules comprise a first RNA molecule comprising a sequence that encodes a heavy chain variable region (VH) and a second RNA molecule comprising a sequence that encodes a light chain variable region (VL);
the at least two DNA/RNA duplexes comprise a first DNA/RNA duplex comprising a sequence that encodes a heavy chain variable region (VH) and a second DNA/RNA duplex comprising a sequence that encodes a light chain variable region (VL); and the at least two cDNA transcripts comprising the second unique DNA barcode comprise a first cDNA transcript comprises a sequence that encodes a VH and the second unique DNA barcode sequence and a second cDNA transcript comprises a sequence that encodes a light chain variable region (VL) and the second unique DNA barcode sequence.

According to one further particular item, the second cell is an immune cell; the at least two RNA molecules comprise a first RNA molecule comprising a sequence that encodes a T-cell Receptor α-chain (TCR-α) and a second RNA molecule comprising a sequence that encodes a T-cell Receptor β-chain (TCR-β);
the at least two DNA/RNA duplexes comprise a first DNA/RNA duplex comprising a sequence that encodes a TCR-α and a second DNA/RNA duplex comprising a sequence that encodes a TCR-β; and
the at least two cDNA transcripts comprising the first unique DNA barcode comprise a first cDNA transcript comprises a sequence that encodes a TCR-α and the first unique DNA barcode sequence and a second cDNA transcript comprises a sequence that encodes TCR-β and the first unique DNA barcode sequence.

According to one further item, the second unique DNA barcode sequence comprises a DNA sequence comprising, from 5′ to 3′, a second unique barcode sequence, wherein the barcode sequence is single- or double-stranded and a second priming sequence for reverse-transcription, wherein the priming sequence is single-stranded.

According to one item, the first lysing composition and the second lysing composition are identical.

According to one item, the first unique DNA barcode sequence and the second unique DNA barcode sequence are different.

According to one item, the volume of the first aqueous composition is between about 10 and 5000 picoliters, inclusive of the endpoints. For example, the volume of the first aqueous composition may be 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0 nanoliters or any amount in between.

According to one particular item, the volume of the first aqueous composition has has a volume of less than 3 nL. According to a more particular item, the volume of the first aqueous composition has a volume of less than 2.5 nL, less than 2 nL, less than 1.5 nL, less than 1 nL, less than 0.5 nL, for example 0.1 nL to 3 nL, 0.5 nL to 3 nL, 1 nL to 3 nL, typically, 0.1 nL, 0.5 nL, 1 nL, 1.2 nL, 1.4 nL, 1.6 nL, 1.8 nL, 2.0 nL, 2.2 nL, 2.4 nL, 2.6 nL, 2.8 nL, 3 nL.

According to one item, the volume of the second aqueous composition is between about 10 and 5000 picoliters, inclusive of the endpoints. For example, the volume of the second aqueous composition of the disclosure may be 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0 nanoliters or any amount in between.

According to one particular item, the volume of the second aqueous composition has a volume of less than 3 nL. According to a more particular item, the volume of the second aqueous composition has a volume of less than 2.5 nL, less than 2 nL, less than 1.5 nL, less than 1 nL, less than 0.5 nL, for example 0.1 nL to 3 nL, 0.5 nL to 3 nL, 1 nL to 3 nL, typically, 0.1 nL, 0.5 nL, 1 nL, 1.2 nL, 1.4 nL, 1.6 nL, 1.8 nL, 2.0 nL, 2.2 nL, 2.4 nL, 2.6 nL, 2.8 nL, 3 nL.

According to one item, the concentration of the first unique DNA barcode sequence in the first aqueous composition is at least or preferably more than 100 nM.

According to one item, the concentration of the second unique DNA barcode sequence in the second aqueous composition is at least or preferably more than 100 nM.

According to one item, the first lysing composition and/or the second lysing composition comprise magnesium chloride, a detergent, a buffered solution, and an RNAse inhibitor.

According to one item, the magnesium chloride is at a concentration of between 1 mM to 20 mM, inclusive of the endpoints.

According to one item, the detergent is selected from the group consisting of Triton-X-100, Nonidet P40, and Tween-20.

According to one item, the detergent is at a concentration of 0.1% to 10%, inclusive of the endpoints.

According to one item, the buffered solution is selected form the group consisting of Tris-HCl, Hepes-KOH and Pipes-NaOH.

According to one further item, the RNase inhibitor is selected from the group consisting of RNase OUT, IN, and SuperlN RNase.

According to one item, the first reverse transcriptase composition and/or the second reverse transcriptase composition comprise(s) a protease inhibitor, dNTPs and DTT.

According to one item, the protease inhibitor comprises a plurality of protease inhibitors.

According to one item, the protease inhibitor comprises one or more of Leupeptin hemisulfate salt, pepstatin A, AEBSF, Aprotinin, Bestatin hydrochloride, E-64 and PMSF.

According to one item, the reverse transcriptase enzyme is Superscriptase I, Superscriptase II, Superscriptase Ill, Superscriptase IV, Moloney Leukemia RT, SmartScribe RT or MultiScribe RT.

According to one item, removing unincorporated DNA barcode sequences comprises contacting the first aqueous composition to a purification substrate wherein the purification substrate removes the unincorporated DNA barcode sequences.

According to another item, removing unincorporated DNA barcode sequences comprises contacting the second aqueous composition to a purification substrate wherein the purification substrate removes the unincorporated DNA barcode sequences.

According to one item, the purification substrate comprises beads or particles.

According to one item, the beads or particles form a column.

According to one item, sequencing each of the at least two cDNA transcripts comprises performing a next generation sequencing (NGS) protocol on a sequencing library.

According to one item, the NGS protocol comprises loading an amount of the sequencing library between 8 μM to 14 μM per flow cell of a reagent kit.

According to one item, the NGS sequencing protocol further comprises the step of adding 10-25% PhiX to the amount of the sequencing library or to the flow cell of the reagent kit.

According to one item, the methods of the invention further comprise inserting the sequence of the first cDNA transcript and the sequence of the second cDNA transcript into an expression vector.

The term “Vector” as used in context of these items includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells.

According to one item, the methods of the invention further comprise the steps of contacting the first cDNA transcript and the second cDNA transcript to a sequencing library and amplifying at least two sequences of interest from the sequencing library that correspond to the first cDNA transcript and the second cDNA transcript, respectively.

According to one item, the methods of the invention further comprise inserting the first sequence of interest corresponding to the first cDNA transcript and the sequence of interest corresponding to the second cDNA transcript into an expression vector.

According to one item, the methods of the invention further comprise expressing the expression vector in a host cell, thereby producing a recombinant polypeptide.

According to one item, the host cell is a mammalian cell.

According to one item, the mammalian cell s a 293, 293T, HeLa, CHO or U2OS cell.

According to one item, the first aqueous composition is a droplet.

According to one item, the first aqueous composition is contained in a first container or any compartment thereof.

According to one item, the first container or any compartment thereof is a plate, well, tube, channel, nano well, nano tube, or nano channel.

According to one item, the second aqueous composition is a droplet.

According to one item, the second aqueous composition is contained in a second container or any compartment thereof.

According to one item, the second container or any compartment thereof is a plate, well, tube, channel, nano well, nano tube, or nano channel.

According to one item, the first container has a maximal volume of between 3 to 5 nanoliters, inclusive of the endpoints.

According to one item, the first container and/or the second container has a maximal volume of between 3 to 5 nanoliters, inclusive of the endpoints.

According to one item, the first carrier is a solid support or a liquid carrier.

According to one item, the solid support is a bead, a hydrogel bead, a polystyrene bead, a magnetic particle, or a surface.

According to one item, the liquid carrier is an emulsion, a solution, a buffer, an aqueous solution, a non-aqueous solution.

According to one item, the second carrier is a solid support or a liquid carrier.

According to one item, the solid support is a bead, a hydrogel bead, a polystyrene bead, a magnetic particle, or a surface.

According to one item, the liquid carrier is an emulsion, a solution, a buffer, an aqueous solution, a non-aqueous solution.

Throughout the instant application, the term “and/or” is a grammatical conjunction that is to be interpreted as encompassing that one or more of the cases it connects may occur.

For example, the wording “error detection and/or correction” in the phrase “the sequences allow for error detection and/or correction” indicates that the sequences may allow for error detection and the sequence may allow for error correction or the sequences may allow for error detection or the sequence may allow for error correction.

As used in this specification and the appended claims, the singular forms “a,” “an” and “the” include plural references, such as a plurality of the object referred to, unless the content clearly dictates otherwise.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

In the entire description, features described in one section are entirely applicable to other sections of the instant description. For instance, the description referring to “primers” as given in the section “Method for capturing and barcoding single cell nucleic acid” is entirely applicable to the section called “Plurality of microfluidic droplets” and the section called “A method for preparing a plurality of microfluidic droplets” or, for example, the description referring to “at least one type of oligonucleotide” as given in the section “Method for capturing and barcoding single cell nucleic acid” is entirely applicable to the section called “Plurality of microfluidic droplets” and the section called “A method for preparing a plurality of microfluidic droplets”.

The invention will now be described in more detail with reference to the following examples. All literature and patent documents cited herein are hereby incorporated by reference. While the invention has been illustrated and described in detail in the the foregoing description, the examples are to be considered illustrative or exemplary and not restrictive.

### FIGURES

FIG. 1A is a graph demonstrating the Cp value (i.e. the crossing point value determined by the 2^{nd} derivative max calculation method) of three human genes (RPS29, ATP5G3 and ZNF480A) across three different conditions (purified RNA, cells lysed in bulk, and cells lysed in a drop) using a non-optimized protocol.

FIG. 1B is a graph depicting the percentage of the mean efficiency (of the genes shown in FIG. 1A) using a non-optimized protocol of reverse transcription across three different conditions (purified RNA, Cells lysed in bulk, and Cells lysed in a drop).

FIG. 2 is a graph depicting the percentage of the mean efficiency (of the genes shown in FIG. 1A) when compared to purified RNA in three conditions varying the total volume of the reaction used per cell using a non optimized protocol.

FIG. 3A is a graph depicting the changes in Cp value by increasing the reverse transcriptase (RT) concentration in a droplet-based experiment across three different genes (RPS29, ATP5G3 and ZNF480A).

FIG. 3B is a graph depicting the impact of lysis buffer composition for efficient cDNA synthesis in a droplet-based experiment across two different genes (RPS29, ATP5G3).

FIG. 3C is a graph recapitulating the impact of the changes in RT amount (FIG. 3A) and lysis conditions (FIG. 3B) in the efficiency of cDNA synthesis for the ATP5G3 gene.

FIG. 3D is a graph depicting the further impact of both increased drop volume and changes in the reverse transcription (RT) conditions in the efficiency of reverse transcription, measured by the Cp value for ATP5G3 gene. In this experiment, the amount of RT enzyme and RNase inhibitor were increased and the lysis buffer was altered.

FIG. 3E is a graph depicting the percentage of RT efficiency of droplet-based approaches compared to purified RNA. The percentage of RT efficiency is above 100% for the immunoglobulin light chain variable genes and approximately 30% for the immunoglobulin heavy chain variable genes. The lower value for VH may be due to lower PCR efficiency on barcoded sample.

FIG. 4A is a series of gels depicting the results of a nested polymerase chain reaction (PCR) amplification of barcoded single cell transcripts encoding immunoglobulin variable heavy (VH) and variable light (VL) chains. The top gel is the first PCR reaction (VH amplicon on the left and VL amplicon on the right), the gel in the middle is the second PCR (VH amplicon on the left and VL amplicon on the right) and the bottom gel is the QC of the final PCR sequencing library product confirming the presence of Illumina NGS P5/P7 sequences and the presence of Light and Heavy chain associated barcode in the corresponding sequencing libraries.

FIG. 4B is a graph depicting paired antibody sequence reads sharing the same barcode (i.e. coming from the same cells) plotted according to the number of VH reads (x axis) and VL reads (y axis).

FIG. 4C is a graph depicting the rate of correct hybridoma VH/VL pairing as a function of read threshold for each VH/VL chain.

FIG. 4D is a chart depicting the number of hybridoma VH/VL pairs as a function of read threshold (for both chain) FIG. 5A is a pair of gels depicting the results of a nested polymerase chain reaction (PCR) amplification of barcoded single cell VH/VL.

FIG. 5B is a graph depicting the number of VH and VL reads in the paired sequences.

FIG. 5C is a graph depicting the number of hybridoma VH/VL pairings as a function of read threshold (for both VH and VL chains).

FIG. 5D is a graph depicting the number of correct hybridoma VH/VL pairings as a function of read threshold (for both VH and VL chains) and droplet volume and cDNA purification method.

FIG. 5E is a graph depicting the distribution of recovered hybridoma pairs at a threshold of 10 reads per chain depending on droplet volume and purification method.

FIG. 6A is a graph depicting the number of VH/VL pairings from sorted B cells as a function of read threshold (for both VH and VL chains).

FIG. 6B is a graph depicting the number of distinct CDR3 sequences identified as a function of read threshold (for both VH and VL chains).

FIG. 6C is a graph depicting paired sequence reads sharing the same barcode (i.e., coming from the same cells) plotted according to the number of VH and VL reads.

FIG. 6D is a graph depicting Heavy Variable gene usage in the paired sequences identified at a threshold of 40 reads per chain.

FIG. 6E is a graph depicting Light Kappa Variable gene usage in the paired sequences identified at a threshold of 40 reads per chain.

FIG. 6F is a graph depicting gamma J-germline gene usage in the paired sequences identified at a threshold of 40 reads per chain.

FIG. 6G is a graph depicting kappa J-germline gene usage in the paired sequences identified at a threshold of 40 reads per chain.

FIG. 7 is a graph depicting capture efficiency of mouse immonuglobulin variable heavy (VH) and variable light (VL) chains reverse transcription for different primer concentrations.

FIG. 8 A) is a graph depicting capture efficiency of mouse immonuglobulin variable heavy (VH) and variable light kappa (VLk) and lambda (VLI) chains reverse transcription for different primer concentrations (10 nM and 100 nM) B) is the picture of the agarose gel loaded with samples of the reverse transcription performed in A)

FIG. 9 is a graph depicting capture efficiency of human housekeeping gene RPS29 and TRAT1 reverse transcription for different primer concentrations (1 μM, 100 nM, 10 nM, 1 nM).

FIG. 10 is a graph depicting the capture efficiency of human housekeeping gene RPS29 and TRAT1 using polydT primer at different concentrations (either 3.3 μM or 100 or 33 nM

### EXAMPLES

In order that the disclosure may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Example 1: Bulk Vs. Drop and RT Efficiency Measurement

Optimization of RT reaction starting from cell has been performed in bulk reaction. This experiment mimics a drop condition (especially the volume of reaction buffer per cell, the amount of reagent per cell). To confirm the bulk conditions mimic the drop conditions, a side-by-side RT reaction was performed in bulk and in drop. To assess the efficiency of the RT reaction, the results were compared with purified RNA extracted from the same cell population.

Comparisons are based on qPCR data. The Cp value (crossing point value determined by the 2^{nd} derivative max calculation method) of the droplet experiment was compared to the bulk experiment as well as to purified RNA samples cDNA synthesis. Droplet based experiments were performed by co-flowing the lysis buffer containing mix (50 μL Lysis buffer 4×; 17.6 μL RT buffer 5×; 22.4 μL H2O; 2 μL polydT oligo 100 μM; 4 μL Protease Inhibitor; 4 μL DY-647 100 μM) with the RT mix buffer (22.5 μL First strand Reaction buffer 5×; 10 μL 0.1M DTT; 10 μL 10 mM each dNTP; 2.5 μL RNAse inhibitor; 2.5 μL Superscript Ill RT enzyme; 2.5 μL DY-647 100 μM solution) with 200,000 human Ramos cells pre-washed in PBS, pelleted and resuspended in 1 μL of DY-647 100 μM solution+49 μL of cell suspension solution (300 μL Percoll; 33.3 μL NaCl 1.5M; 10 μL Pluronic 10%; 25 μL Hepes 1M; 50 μL Serum low IgG 5%; 581.7 μL Media DMEM). Lysis buffer 1× is 50 mM Tris HCl pH7.5, 75 mM NaCl, 3 mM MgCl₂, 0.2% Triton X-100.

Using the feedback module with Fluigent pumps, ensure that the following flow rates are obtained (Table 2).

TABLE 2

| Flow rates (μL/hr) | Reagent |
|---|---|
| Variable, depends on drop volume | 5% (w/w) surfactant in HFE-7500 |
| 75 | RT Mix |
| 75 | Cells |
| 150 | Lysis |
| Flow rates (μL/hr) | Reagent |
| Variable, depends on drop volume | 5% (w/w) surfactant in HFE-7500 |

100 μL drops were generated (with a CV in width of <5%) and emulsion collected on ice in tube filled with HFE-7500. Once the production of the emulsion is finished, a syringe with HFE-7500 was used to attach to the longer tubing of the reservoir. The needle connected at the outlet was filled with oil and the syringe was plugged to prevent formation of any air bubbles. Disconnect immediately the tubing from the chip and slowly pull the plunger out to completely aspirate the emulsion still presents in the tubing. Avoid air bubbles inside the collection tube. The emulsion containing tube was placed into the Thermomixer at 55° C. for 60 min. RT was inactivated at 70° C. for 15 min and then 1 min at 4° C. Emulsion was collected and broken using perfluoro-octanol (v/v of emulsion), incubated until the oil and aqueous phases are separated. Centrifuge aqueous phase for 10 min at 10,000 g at 4° C. and recover the supernatant. Digest RNA and Protein by incubating with 2.5 μL of RNase A for 15 min at 37° C. and 4 μL of Proteinase K overnight at 37° C.

Bulk experiments were performed by mixing the 200,000 human Ramos cells (pre-washed in PBS as above) or RNA extracted (Machery Nagel extraction kit) from 200,000 human Ramos cells in reaction volume mimicking 100 μL per cell (20 μL final reaction). The mix is shown in Table 3.

TABLE 3

| | 100 pl | |
|---|---|---|
| | per cell | |
| H₂O RNase/DNase free | 5.9 | μL |
| Cell Suspension Buffer | 5 | μL |
| (Containing Cells) | | |
| 10x Lysis Buffer | 2 | μL |
| 5x RT Buffer | 4 | μL |
| dNTP 10 mM | 1 | μL |
| PolydT primer 100 μM stock | 0.2 | μL |
| 0.1M DTT | 1 | μL |
| SS III RT enzyme (200 U/μL) | 0.25 | μL |
| RNase Inhibitor | 0.25 | μL |
| Protease Inhibitor | 0.4 | μL |
| Final volume | 20 | μL |

The mixed cells and RNA were incubated for 10 min on ice and then RT was performed for 1 h at 55° C. RT was inactivated at 70° C. for 15 min. cDNA were processed as above.

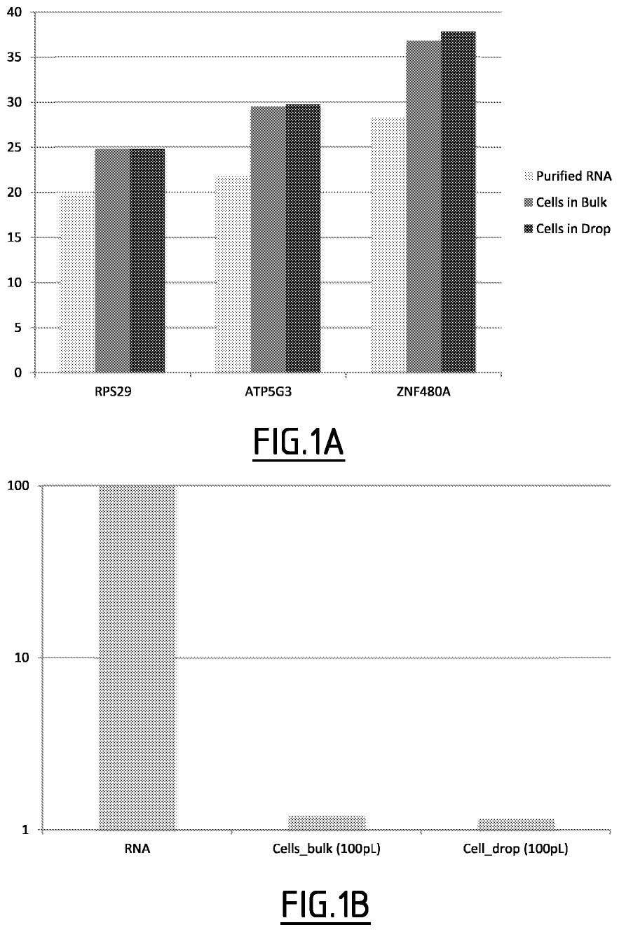

All cDNAs were purified using RNAClean XP Beckman (at 0.8× ratio) and eluted in 20 μL H₂O. cDNAs were diluted 3 times and used as template for qPCR reaction (Roche LightCycler 480 master mix) for 3 sets of housekeeping genes known to be expressed at different level in Ramos cells. Cp values from pure RNA conditions and cells reactions were compared (FIG. 1A) and then used to estimate RT efficiency (RNA samples were considered as 100% efficient; FIG. 1B).

Altogether this experiment shows droplet and bulk based experiment mimicking the same volume/cell give comparable results for the 3 genes tested (FIG. 1A). Second, using standard conditions, mean of RT efficiency is approximately 2% for the 3 genes tested (FIG. 1 B). This value is in agreement with previous reports.

Antisense primer sequence for human genes qPCR
Top_SBS12-ATP5G3:
(SEQ ID NO: 1)
/5Phos/CAACGTGACTGGAGTTCAGACGTGTGCTCTTCCGATCTCTGCT
TCAGCGAAGGGTTTC
Top_SBS12-RPS29:
(SEQ ID NO: 2)
/5Phos/CAACGTGACTGGAGTTCAGACGTGTGCTCTTCCGATCTACAGA
CACGACAAGAGCGA
Top_SBS12-ZNF780A
(SEQ ID NO: 3)
/5Phos/CAACGTGACTGGAGTTCAGACGTGTGCTCTTCCGATCTTGATC
CATGGACCATGTTGCT
Sense primer sequence for human genes qPCR
SBS3-ATP5G3:
(SEQ ID NO: 4)
ACACTCTTTCCCTACACGACGCTCTTCCGATCTCAGGTGCTGCAACAGTA
GGA
SBS3-RPS29:
(SEQ ID NO: 5)
ACACTCTTTCCCTACACGACGCTCTTCCGATCTTTACCTCGTTGCACTGC
TGA
SBS3-ZNF780A:
(SEQ ID NO: 6)
ACACTCTTTCCCTACACGACGCTCTTCCGATCTAGAGCGTTACTGCTGCA
CA

### Example 2: Impact of Drop Size in RT Efficiency

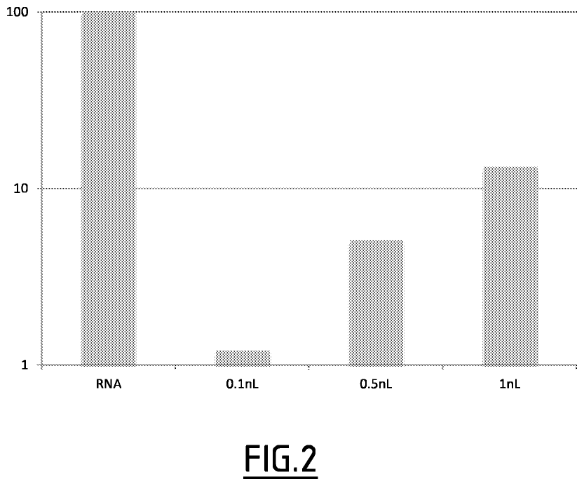

The impact of buffer volume per cell in RT efficiency was assessed by increasing the reagent/volume accordingly to Example 1. The Cp value was compared to purified RNA conditions (FIG. 2). A 1% RT efficiency in 100 μL volume was achieved, which is higher than reported and increased to >10% efficiency in 1 nL reaction/cell.

### Example 3: Impact of Amount of RT Enzyme, Lysis Buffer in RT Efficiency

To further improve the RT efficiency in sub-nanoliter volumes, the impact of increased RT concentration, lysis buffers and reagents of the RT reaction were assessed. We compared the impact of these modifications on the RT efficiency, based on purified RNA conditions in a standard cDNA synthesis (as described by supplier).

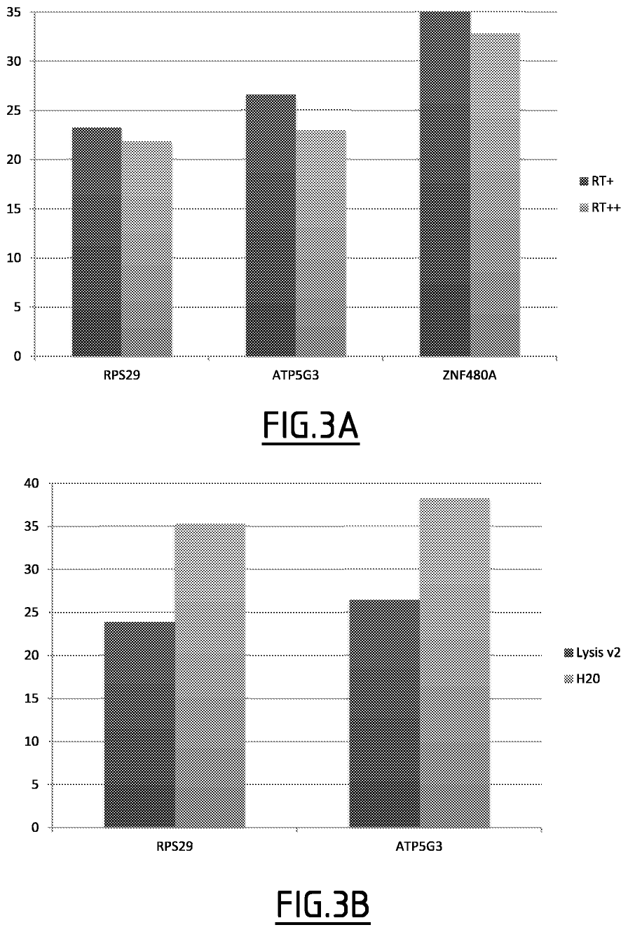

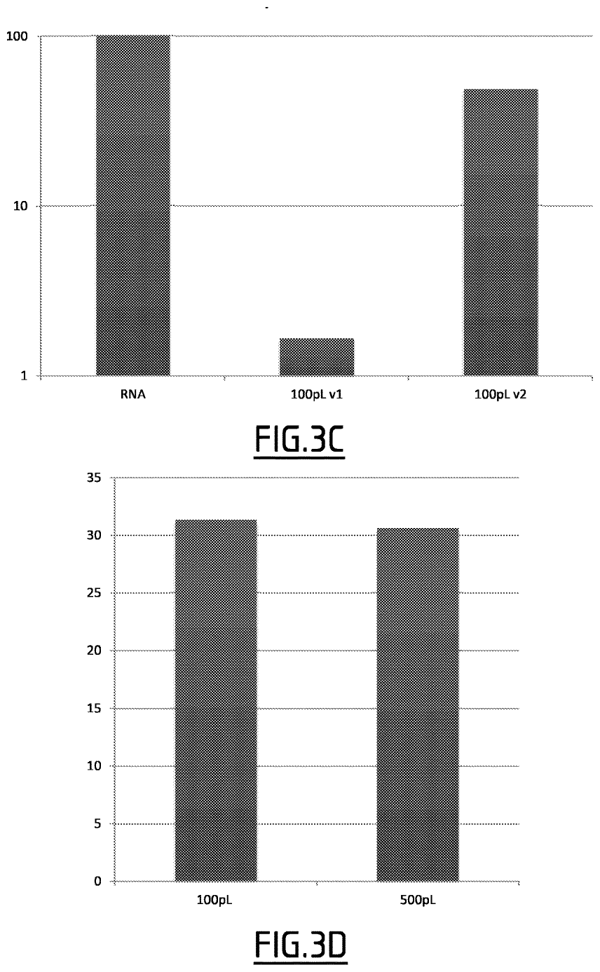

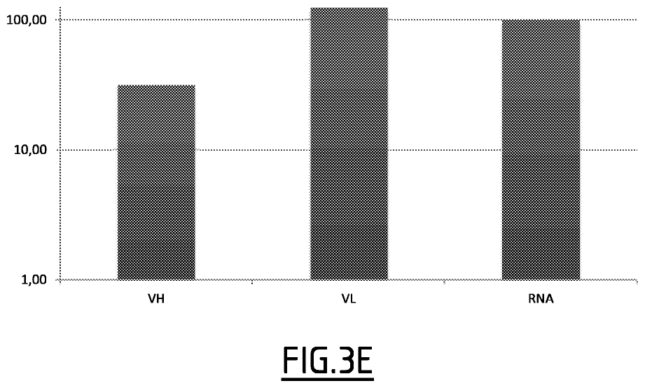

FIG. 3A shows the impact of increasing the RT concentration in droplet-based experiments. Increasing the RT enzyme by a factor of 5 increased the cDNA synthesis by a mean of 10 times (based on 3 different genes Cp values). The biggest impact is achieved for low expressed genes, arguing for a genuine effect of RT synthesis efficiency.

The microfluidic encapsulation of cells together with RT primers and RT mix was performed as described in Example 1. Droplet based experiment were performed by co-flowing the lysis buffer containing mix (50 μL Lysis buffer 4×; 17,6 μL RT buffer 5×; 22.4 μL H₂O; 2 μL polydT oligo 100 μM; 4 μL Protease Inhibitor; 4 μL DY-647 100 μM) with the RT mix buffer (22.5 μL First strand Reaction buffer 5×; 10 μL O. M DTT; 10 μL 10 mM each dNTP; 2.5 μL RNAse inhibitor; 2.5 μL Superscript Ill RT enzyme; 2.5 μL DY-647 100 μM solution) with 200,000 human Ramos cells pre-washed in PBS, pelleted and resuspended in 1 μL of DY-647 100 μM solution+49 μL of cell suspension solution (300 μL Percoll; 33.3 μL NaCl 1.5M; 10 μL Pluronic 10%; 25 μL Hepes 1M; 50 μL Serum low IgG 5%; 581.7 μL Media DMEM). Lysis buffer 1× is 50 mM Tris HCl pH7.5, 75 mM NaCl, 3 mM MgCl₂, 0.2% Triton X-100.

In the emulsion where the concentration of RT enzyme is 5× higher than in the example above, we co-flowed the lysis buffer containing mix (50 μL Lysis buffer 4×; 27.6 μL RT buffer 5×; 12.4 μL H2O; 2 μL polydT oligo 100 μM; 4 μL Protease Inhibitor; 4 μL DY-647 100 μM) with the RT mix buffer (12.5 μL First strand Reaction buffer 5×; 10 μL 0.1M OTT; 10 μL 10 mM each dNTP; 2.5 μL RNAse inhibitor; 12.5 μL Superscript IL RT enzyme; 2.5 μL DY-647 100 μM solution) and proceeded to cell encapsulation as above and as Example 1. cDNA purification and qPCR was done as Example 1. FIG. 3B shows the impact of lysis buffer for efficient cDNA synthesis. MgCl₂ complemented (which is required for efficient RT enzyme activity) hypotonic lysis buffer was compared with hypotonic water solution. MgCl_{z} complemented hypotonic lysis buffer is required for efficient cDNA synthesis in sub nanoliter reaction volumes per cell. As described in Example 1, the impact of lysis condition was analyzed starting from cells in a bulk reaction and in a volume mimicking 100 μL per cell. Bulk experiments were performed by mixing the 200,000 human Ramos cells (pre-washed in PBS as Example 1) in a reaction volume mimicking 100 μL per cell (20 μL final reaction). The mix is described below in Table 4.

TABLE 4

| | | | Lysis in MgCl₂ | | Lysis in | |
|---|---|---|---|---|---|---|
| | Reagent final | | supplemented | | hypotonic | |
| | concentration | | hypotonic buffer | | H₂O solution | |
| H₂O RNase/DNase | | | 1.7 | μL | 3.7 | μL |
| free | | | | | | |
| Cell Suspension | | | 5 | μL | 5 | μL |
| Buffer (Containing | | | | | | |
| 200,000 Cells) | | | | | | |
| 10x Lysis Buffer | 1x | | 2 | μL | — | |
| (100 mM TrisHCl | | | | | | |
| pH 7.5, 33.3 mM | | | | | | |
| MgCl₂, | | | | | | |
| 2% TritonX100) | | | | | | |
| 5x RT Buffer | 1x | | 4 | μL | 4 | μL |
| dNTP 10 mM | 0.5 | mM | 1 | μL | 1 | μL |
| 0.1M DTT | 5 | mM | 1 | μL | 1 | μL |
| SS III RT enzyme | 12.5 | U/μL | 1.25 | μL | 1.25 | μL |
| (200 U/μL) | | | | | | |
| RNase Inhibitor | 1.25 | U/μL | 1.25 | μL | 1.25 | μL |
| Protease Inhibitor | 2x | | 0.8 | μL | 0.8 | μL |
| 10 μM polydT oligo | 1 | μM | 2 | μL | 2 | μL |
| Final volume | | | 20 | μL | 20 | μL |

Cels in the reverse transcriptase (RT) reaction mix were first incubated for 10 min on ice in order to lyse the cells. The RT was then triggered by incubating the reaction for 1 h at 55° C. (shaking at 750 rpm). RT enzyme were inactivated at 70° C. for 15 min. Cell debris was pelleted by centrifuging the RT reaction for 10 min at 10,000 g 4° C. cDNA in suspension were recovered in the supernatant, treated sequentially with 2.5 μL RNase A for 15 min at 37° C. (shaking at 750 rpm) and with 4 μL Proteinase K for ON at 37° C. (shaking at 750 rpm). cDNA was purified using RNA clean beads (Beckman) at 0.8× beads/solution ratio and eluted in 20 μL of DNase and RNase free water. Purified cDNA was used for assessing cDNA synthesis efficiency by qPCR.

FIG. 3C recapitulates the impact of the changes in RT amount/lysis conditions in the efficiency of cDNA synthesis. The overall RT efficiency was improved by approximately 40 times (from 1.2% to above 49%; purified RNA was set at 100%).

FIG. 3D shows the further impact of both increase drop volume and changes in the RT conditions (increase amount of RT enzyme and of RNase inhibitor and changes in the lysis buffer) in the RT efficiency. A side-by-side droplet based single cell RNAseq experiment was performed encapsulating mouse hybridoma cells either in 100 μL or 500 μL drops, as described in Examples 5. RT efficiency analysis of captured ATP5G3 housekeeping gene barcoded cDNA showed a 2-fold increase in the amount of cDNA generated by the 500 μL droplet based RNAseq compared to the 100 μL. This result has been consistently repeated with human cells for other genes and in the mouse hybridoma system.

Antisense primer sequence for mouse ATP5G3
gene qPCR
(SEQ ID NO: 7)
/5Phos/CAACTGCAACTCTGGATCCAGCTC
Sense primer sequence for mouse ATP5G3
gene qPCR
(SEQ ID NO: 8)
ACACTCTTTCCCTACACGACGCTCTTCCGATCTTCGCCTGTCACCTAGAT
CCA

To reach 100% RT efficiency compared to purified RNA conditions, the RT reaction conditions were further optimized (Percoll removal during cell encapsulation, selected broad spectrum RNAse inhibitor, optimization of cDNA recovery and purification). Mouse hybridoma cells expressing immunoglobulins were encapsulated in 500 μL drops and performed droplet based single cell VH\NL mRNA capture and cDNA synthesis in the drop. In parallel, RNA were extracted and purified from the same cells population and reverse transcription of VH and VL mRNA was performed in tube based assay, as described by supplier recommendation. The VH\NL cDNA synthesis efficiency was assessed by real time quantitative PCR. FIG. 3E shows the % of RT efficiency of droplet based approaches compared to purified RNA, which is above 100% for the light chain immunoglobulin variable genes and approximately 30% for the heavy chain immunoglobulin variable genes. The lower value for VH may be due to lower PCR efficiency on barcoded sample.

Cells were washed in Cell Wash Buffer (CWB; DMEM F12 media, 0.1% Pluronic, 25 mM Hepes, 5% low IgG serum) and co-flowed in the microfluidic design in 47.4 μL CWB buffer containing 2.6 μL 100 μM DY-647 solution. The hydrogel beads library carrying the single cell barcodes and the primers for VH and VL mRNA capture and cDNA synthesis in the drop where washed 10 times in 1×BW buffer, spin at 2500 g for 2 min at 4° C. The double stranded barcodes were denatured in 1 mL denaturation solution (700 μL H₂O+300 μL 1M NaOH) at 22° C. for 2 min. The beads were washed 3 times in BW buffer, and labeled with 10 μL of FITC biotin 100 μM and incubated at room temperature on a rotating platform for 10 min. Following 3 washes in BW buffer, the beads were heated for 2 min at 70° C. and the supernatant removed. Finally the beads were washed in 200 μL of the lysis/Pi/DTT/Dye containing buffer (80 μL Lysis buffer 10×; 8 μL Protease inhibitor, 8 μL Dye 647 100 μM and 4 μL 1M DTT). The beads were spun at 2500 g for 2 min at 4° C. and the supernatant was removed leaving 50 μL of buffer/HgB mix.

Droplet based experiments were performed by co-flowing the hydrogel beads carrying single cell barcode and primers in lysis buffer with the RT mix buffer (52 μL First strand Reaction buffer 5×; 10 μL 0.1M DTT; 13 μL 10 mM each dNTP; 16.25 μL SUPERin RNAse inhibitor; 16.25 μL Superscript Ill RT enzyme; 2.5 μL DY-647 100 μM solution) with 15,000 mouse hybridoma cells.

The encapsulation of cells together with RT primers and RT mix was performed as shown in Table 5.

TABLE 5

| Flow rates (μL/hr) | Reagent |
|---|---|
| 200-250 | 5% (w/w) surfactant in HFE-7500 |
| 74 (200 for a Syringe pump) | RT Mix |
| 50 (200 for a syringe pump) | Hydrogel beads in lysis buffer |
| 74 (100 for a syringe pump) | Cells |

### Example 4: Hybridoma Mixture

To assess VH/VL gene pairing accuracy in sub-nanoliter droplet, mouse hybridoma cell lines expressing known antibody sequences were mixed in equal proportions. The cells were washed in PBS and resuspended in 47.5 μL of Cell Suspension Buffer (300 μL Percoll; 33.3 μL NaCl 1.5M; 10 μL Pluronic 10%; 25 μL Hepes 1M; 50 μL Serum low IgG 5%; 581.7 μL Media DMEM) plus 2.5 μL of 100 μM DY-647.

Cells were co-encapsulated with hydrogel beads library carrying the single cell barcodes and the primers for VH and VL mRNA capture and cDNA synthesis in the drop.

| | RT primer sequence: |
|---|---|
| | VH_1: |
| (SEQ ID NO: 9) | |
| | GGCCAGTGGATAGACAGATGGGGG |
| | VH_2: |
| (SEQ ID NO: 10) | |
| | GGCCAGTGGATAGACCGATGGGGC |
| | VH_3: |
| (SEQ ID NO: 11) | |
| | GGCCAGTGGATAGACTGATGGGGG |
| | VH_4: |
| (SEQ ID NO: 12) | |
| | GTCACCGCAGCCAGGGACCAAGGG |
| | VLk_1: |
| (SEQ ID NO: 13) | |
| | GCGTTTCATTTCCAGCTTGG |
| | VLk_2: |
| (SEQ ID NO: 14) | |
| | GCGTTTGATTTCCAGCTTGG |
| | VLk_3: |
| (SEQ ID NO: 15) | |
| | GCGTTTTATTTCCAATTTTG |

The hydrogel beads where washed 10 times in 1×BW buffer and spun at 2500 g for 2 min at 4° C. The double stranded barcodes were denatured in 1 mL denaturation solution (700 μL H₂O+300 μL 1M NaOH) at 22° C. for 2 min. The beads were washed 3 times in BW buffer, and labeled with 10 μL of FITC biotin 20 μM and incubate at room temperature on a rotating platform for 1 h. Following 3 washes in 4× lysis buffer (0.8% TritonX100, 12 mM MgCl₂, 200 mM Tris-HCl pH7.4), the beads were heated for 2 min at 70° C. and the supernatant removed. The beads were solubilized in 50 μL lysis buffer 4×, 27.6 μL of 5× first strand RT buffer, 4 μL Protease Inhibitor, 4 μL DY-647 100 μM, 4.4 μL H₂O and 10 μL 0.1M DTT.

The beads and the cells were co-flowed with RT mix described in Table 6.

TABLE 6

| | Volume |
|---|---|
| Mix | [μL] |
| 5x SuperScript III RT buffer | 12.5 |
| dNTP 10 mM | 10 |
| RNase inhibitor (20 U/μL) | 12.5 |
| SuperScript III RT (200 U/μL) | 12.5 |
| 100 μM DY-647 solution | 2.5 |

Using the feedback module with Fluigent pumps, ensures that the following flow rates shown in Table 7 were obtained.

TABLE 7

| Flow rates | |
|---|---|
| (μl/hr) | Reagent |
| 150 | 5% (w/w) surfactant in HFE-7500 |
| 75 | RT Mix |
| 75 | Cells |
| 150 | Hydrogel beads in lysis buffer |

Ensure producing drops of 100-110 μL. Once the emulsion was produced, oligo photocleavage proceeded for 30 s at 200 mW/cm². The emulsion was incubated at 55° C. for 60 min and the RT was inactivated at 70° C. for 15 min. The tube was allowed to cool down for 1 min at 4° C. The Emulsion was transferred by pushing the syringe plunger in a new 1.5 mL DNA LoBind tube and vortex 30 s the emulsion to allow oligo diffusion from beads. To efficiently break the emulsion and recover the cDNA, as much oil was removed as possible with a syringe without taking any drops. Perfluorooctanol (v/v of emulsion) was added and allowed to mix for 5-10 min at room temp with occasional soft vortex (5 seconds) and soft spin (5 seconds) to separate phases.

A second photocleavage proceeded for 60 s at 200 mW/cm². The emulsion was vortexed for 30 s and incubated for 5 min at room temperature to allow oligo diffusion from beads.

The oil phase was removed with a syringe. The emulsion was vortexed 30 s and incubated for 5 min at room temperature to allow oligo diffusion from beads. cDNA was treated with 2.5 μL of RNase A to the Eppendorf and placed in a DNase/RNase free DNA LoBind tube for 15 min at 37° C. The cDNA solution was treated with 4 μL of Proteinase K and placed in a 1.5 ml DNase/RNase free DNA LoBind tube for 1 h at 50° C. or overnight at 37° C.

The aqueous phase containing cDNA and hydrogel beads was applied to a 30 μM filter (Pierce Spin column) and washed with H₂O. 25% of the filtered cDNA was purified using RNA Clean UP beads with a 1× ratio and eluted into 40 μL of DNase and RNase free water. The VH\NL PCR reaction was performed as follows:

Use 4*10 μL of purified cDNA to perform PCR1 as described in Tables 8A and 8B.

TABLE 8A

| Mix | 1 tube | Final conc. | | N tubes |
|---|---|---|---|---|
| H₂O | 14.92 | — | | 119.36 |
| 5X Go Taq Buffer | 10 | 1x | | 80 |
| MgCl2 | 3 | 1.25 | mM | 24 |
| dNTP 10 mM | 1 | 0.2 | mM | 8 |
| Primer as - 5 μM | 2.5 | 0.25 | μM | |
| Primer s - 1.5 μM | 8.33 | 0.25 | μM | 66.64 |
| Go Taq Polymerase | 0.25 | | | 2 |

| | | | |
|---|---|---|---|
| DNA template | 10 | — | |
| Total | 50 | — | 300 |

TABLE 8B
VH and VL
Thermo-cycling

| | | | Temps | | |
|---|---|---|---|---|---|
| | Time | | (°C) | | |
| Activate | 2 | min | 95 | | |
| Taq Polymerase: | | | | | |
| Denaturation: | 1 | min | 95 | | |
| Annealing: | 1 | min | 55 | {close oversize bracket} | 25 cycles |
| Extension: | 1 | min | 72 | | |
| Finishing: | 10 | min | 72 | | |

| | |
|---|---|
| Keep | 4 °C |

The PCR was purified using AMPure XP beads at a ratio of 1× and eluted in 20 μL water. 10% of the PCR was run a 2% agarose gel. 50% of the purified PCR1 was run the PCR2 as follow shown in Tables 9A and 91B.

TABLE 9A

| Mix | 1 tube | Final conc. | | N tubes |
|---|---|---|---|---|
| H₂O | 17 | — | | 136 |
| 5X Go Taq Buffer | 10 | 1x | | 80 |
| MgCl2 | 3 | 1.25 | mM | 24 |
| dNTP 10 mM | 1 | 0.2 | mM | 8 |
| Primer as - 5 μM | 2.5 | 0.25 | μM | |
| Primer s - 1.5 μM | 6.25 | 0.25 | μM | 50 |
| Go Taq Polymerase | 0.25 | | | 2 |

| | | | |
|---|---|---|---|
| DNA template | 10 | — | |
| Total | 50 | — | 300 |

TABLE 9B
VH and VL
Thermo-cycling

| | | | Temps | | |
|---|---|---|---|---|---|
| | Time | | (°C) | | |
| Activate | 2 | min | 95 | | |
| Taq Polymerase: | | | | | |
| Denaturation: | 1 | min | 95 | | |
| Annealing: | 1 | min | 55 | {close oversize bracket} | 10 cycles |
| Extension: | 1 | min | 72 | | |
| Finishing: | 10 | min | 72 | | |

| | |
|---|---|
| Keep | 4 °C |

The PCR was purified using AMPure XP beads at a ratio of 1× and eluted in 20 μL water. 10% of the PCR was run a 2% agarose gel. The gel was cut and the bands were weighed. Bands were purified using a Gel Extraction kit (QIAGEN™) and eluted in 10 μL water.

### PCR Primer Set

### First PCR Reaction

| | T7 antisense PCR1 primer sequence |
|---|---|
| (SEQ ID NO: 16) | |
| | GAATTTAATACGACTCACTATAGGGAGA |

TABLE 10
VH sense PCR_1 (v2) primer sequence

| MmLH_AG1 | TAACTGCAGGTGTCCACTCC (SEQ ID NO: 17) |
|---|---|
| MmLH_AG2 | CAGCTACAGGTGTCCACTCC (SEQ ID NO: 18) |
| MmLH_AG3 | TTTATCAAGGTGTGCATTGT (SEQ ID NO: 19) |
| MmLH_AG4 | GAACTGCAGGCGTCCACTCT (SEQ ID NO: 20) |
| MmLH_AG5 | TAACTGCAGGTGTTCACTCC (SEQ ID NO: 21) |
| MmLH_AG6 | TCCCAAGCTGTGTCCTATCC (SEQ ID NO: 22) |
| MmLH_AG7 | TTCCAAGCTGTGTCCTGTCC (SEQ ID NO: 23) |
| MmLH_AG8 | CTTTTAAAGGTATTCACTGT (SEQ ID NO: 24) |
| MmLH_AG9 | TTTTAAAAGGGGTCCAGTGT (SEQ ID NO: 25) |
| MmLH_AG10 | TTTTAAAAGGTGTCCAGTGT (SEQ ID NO: 26) |
| MmLH_AG11 | TTTTAAATGGTATCCAGTGT (SEQ ID NO: 27) |
| MmLH_AG12 | CTGCCCAAAGTGCCCAAGCA (SEQ ID NO: 28) |
| MmLH_AG13 | CTGCCCAAAGTATCCAAGCA (SEQ ID NO: 29) |
| MmLHa | ATGRASTTSKGGYTMARCTKGRTTT (SEQ ID NO: 30) |
| MmLHb | ATGRAATGSASCTGGGTYWTYCTCTT (SEQ ID NO: |
| | 31) |
| MmLHc1 | ATGGACTCCAGGCTCAATTTAGTTTTCCT (SEQ ID NO: |
| | 32) |
| MmLHc2 | ATGGCTGTCYTRGBGCTGYTCYTCTG (SEQ ID NO: |
| | 33) |
| MmLHc3 | ATGGVTTGGSTGTGGAMCTTGCYATTCCT (SEQ ID NO: |
| | 34) |
| MmLHd1 | ATGAAATGCAGCTGGRTYATSTTCTT (SEQ ID NO: |
| | 35) |
| MmLHd2 | ATGGRCAGRCTTACWTYYTCATTCCT (SEQ ID NO: |
| | 36) |
| MmLHd3 | ATGATGGTGTTAAGTCTTCTGTACCT (SEQ ID NO: |
| | 37) |
| MmLHe1 | ATGGGATGGAGCTRTATCATSYTCTT (SEQ ID NO: |
| | 38) |
| MmLHe2 | ATGAAGWTGTGGBTRAACTGGRT (SEQ ID NO: 39) |
| MmLHe3 | ATGGRATGGASCKKIRTCTTTMTCT (SEQ ID NO: 40) |
| MmLHf1 | ATGAACTTYGGGYTSAGMTTGRTTT (SEQ ID NO: 41) |
| MmLHf2 | ATGTACTTGGGACTGAGCTGTGTAT (SEQ ID NO: 42) |
| MmLHf3 | ATGAGAGTGCTGATTCTTTTGTG (SEQ ID NO: 43) |
| MmLHf4 | ATGGATTTTGGGCTGATTTTTTTTATTG (SEQ ID NO: |
| | 44) |

TABLE 11
VL sense PCR_1 (v2) primer sequence

| | |
|---|---|
| MmLKa | ATGRAGWCACAKWCYCAGGTCTTT (SEQ ID NO: 45) |
| MmLKb | ATGGAGACAGACACACTCCTGCTAT (SEQ ID NO: 46) |
| MmLKc | ATGGAGWCAGACACACTSCTGYTATGGGT (SEQ ID NO: |
| | 47) |
| MmLKd1 | ATGAGGRCCCCTGCTCAGWTTYTTGGIWTCTT (SEQ ID |
| | NO: 48) |
| MmLKd2 | ATGGGCWTCAAGATGRAGTCACAKWYYCWGG (SEQ ID |
| | NO: 49) |
| MmLKe1 | ATGAGTGTGCYCACTCAGGTCCTGGSGTT (SEQ ID NO: |
| | 50) |
| MmLKe2 | ATGTTGGGAYCGKTTTYAMMCTTTTCAATTG (SEQ ID |
| | NO: 51) |
| MmLKe3 | ATGGAAGCCCCAGCTCAGCTTCTCTTCC (SEQ ID NO: |
| | 52) |
| MmLKf1 | ATGAGIMMKTCIMTTCAITTCYTGGG (SEQ ID NO: |
| | 53) |
| MmLKf2 | ATGAKGTHCYCIGCTCAGYTYCTIRG (SEQ ID NO: |
| | 54) |
| MmLKf3 | ATGGTRTCCWCASCTCAGTTCCTTG (SEQ ID NO: |
| | 55) |
| MmLKf4 | ATGTATATATGTTTGTTGTCTATTTCT (SEQ ID NO: |
| | 56) |
| MmLKg1 | ATGAAGTTGCCTGTTAGGCTGTTGGTGCT (SEQ ID |
| | NO: 57) |
| MmLKg2 | ATGGATTTWCARGTGCAGATTWTCAGCTT (SEQ ID |
| | NO: 58) |
| MmLKg3 | ATGGTYCTYATVTCCTTGCTGTTCTGG (SEQ ID |
| | NO: 59) |
| MmLKg4 | ATGGTYCTYATVTTRCTGCTGCTATGG (SEQ ID |
| | NO: 60) |
| LL_AG_1 | attgctcaggttctttctcc (SEQ ID NO: 61) |
| LL_AG_2 | cagtcataatgtccagagga (SEQ ID NO: 62) |
| LL_AG_3 | cagtcataatgtccagggga (SEQ ID NO: 63) |
| LL_AG_4 | cagtcatactattcagagga (SEQ ID NO: 64) |
| LL_AG_5 | cagtcatagtgtctaatgga (SEQ ID NO: 65) |
| LL_AG_6 | cagtcatattgaccaatgga (SEQ ID NO: 66) |
| LL_AG_7 | cagtcatattgtccagtgga (SEQ ID NO: 67) |
| LL_AG_8 | cggtatctggtacctgtgga (SEQ ID NO: 68) |
| LL_AG_9 | cgagtccagcctcaagcagt (SEQ ID NO: 69) |
| LL_AG_10 | gaatcacaggcataatatgt (SEQ ID NO: 70) |
| LL_AG_11 | gaatcccaggcatgatatgt (SEQ ID NO: 71) |
| LL_AG_12 | gcatgtctggtgcctgtgca (SEQ ID NO: 72) |
| LL_AG_13 | ggaccacggtctcagctgtc (SEQ ID NO: 73) |
| LL_AG_14 | ggacttcagcctccagatgt (SEQ ID NO: 74) |
| LL_AG_15 | ggatatcaggtgcccagtgt (SEQ ID NO: 75) |
| LL_AG_16 | ggatccctggagccactggg (SEQ ID NO: 76) |
| LL_AG_17 | ggatccctggatccactgca (SEQ ID NO: 77) |
| LL_AG_18 | ggatctctggagtcagtggg (SEQ ID NO: 78) |
| LL_AG_19 | ggattcaggaaaccaacggt (SEQ ID NO: 79) |
| LL_AG_20 | ggattccagcctccagaggt (SEQ ID NO: 80) |
| LL_AG_21 | ggattcctgcttccagcagt (SEQ ID NO: 81) |
| LL_AG_22 | ggattcgggaaaccaacggt (SEQ ID NO: 82) |
| LL_AG_23 | ggatttcagcctccacaggt (SEQ ID NO: 83) |
| LL_AG_24 | ggctccaaggcatgagctgt (SEQ ID NO: 84) |
| LL_AG_25 | ggcttcatggtgctcagtgt (SEQ ID NO: 85) |
| LL_AG_26 | ggcttacagacgcaggatgt (SEQ ID NO: 86) |
| LL_AG_27 | ggcttacagatgccagatgt (SEQ ID NO: 87) |
| LL_AG_28 | gggtatctggtacctgtggg (SEQ ID NO: 88) |
| LL_AG_29 | gggtatctggtgcctgtgca (SEQ ID NO: 89) |
| LL_AG_30 | gggttccaggttccactggt (SEQ ID NO: 90) |
| LL_AG_31 | ggttatatggtgctgatggg (SEQ ID NO: 91) |
| LL_AG_32 | ggttcccaggtgccagatgt (SEQ ID NO: 92) |
| LL_AG_33 | ggttgtctggtgttgaagga (SEQ ID NO: 93) |
| LL_AG_34 | ggtttccaggtatcagatgt (SEQ ID NO: 94) |
| LL_AG_35 | ggtttccaggtgcaagatgt (SEQ ID NO: 95) |
| LL_AG_36 | ggtttgcaggtggtaaatgt (SEQ ID NO: 96) |
| LL_AG_37 | ggtttttaggtgccagatgt (SEQ ID NO: 97) |
| LL_AG_38 | gtgtcacagtgtcaaaggga (SEQ ID NO: 98) |
| LL_AG_39 | gtgtctctgattctagggca (SEQ ID NO: 99) |
| LL_AG_40 | gtgtgtctggtgctcatggg (SEQ ID NO: 100) |
| LL_AG_41 | gttttcaaggtaccagatat (SEQ ID NO: 101) |
| LL_AG_42 | gttttcaaggtaccagatgt (SEQ ID NO: 102) |

### Second Nested PCR Reaction

Illumina Index Antisense primer
(SEQ ID NO: 103)
CAAGCAGAAGACGGCATACGAGATINDEXGTGACTGGAGTTCAGACGTGT
GCTCTTCCGATCT

TABLE 12
VH sense PCR_2 (v2) primer sequence

| | |
|---|---|
| VHs1 | AATGATACGGCGACCACCGAGATCTACACTCTTTCCCTACACGACGCTCTTCCGAT |
| | CTGATGTGAAGCTTCAGGAGTC (SEQ ID NO: 104) |
| VHs2 | AATGATACGGCGACCACCGAGATCTACACTCTTTCCCTACACGACGCTCTTCCGAT |
| | CTCAGGTGCAGCTGAAGGAGTC (SEQ ID NO: 105) |
| VHs3 | AATGATACGGCGACCACCGAGATCTACACTCTTTCCCTACACGACGCTCTTCCGAT |
| | CTCAGGTGCAGCTGAAGCAGTC (SEQ ID NO: 106) |
| VHs4 | AATGATACGGCGACCACCGAGATCTACACTCTTTCCCTACACGACGCTCTTCCGAT |
| | CTCAGGTTACTCTGAAAGAGTC (SEQ ID NO: 107) |
| VHs5 | AATGATACGGCGACCACCGAGATCTACACTCTTTCCCTACACGACGCTCTTCCGAT |
| | CTGAGGTCCAGCTGCAACAATCT (SEQ ID NO: 108) |
| VHs6 | AATGATACGGCGACCACCGAGATCTACACTCTTTCCCTACACGACGCTCTTCCGAT |
| | CTGAGGTCCAGCTGCAGCAGTC (SEQ ID NO: 109) |
| VHs7 | AATGATACGGCGACCACCGAGATCTACACTCTTTCCCTACACGACGCTCTTCCGAT |
| | CTCAGGTCCAACTGCAGCAGCCT (SEQ ID NO: 110) |
| VHs8 | AATGATACGGCGACCACCGAGATCTACACTCTTTCCCTACACGACGCTCTTCCGAT |
| | CTGAGGTGAAGCTGGTGGAGTC (SEQ ID NO: 111) |
| VHs9 | AATGATACGGCGACCACCGAGATCTACACTCTTTCCCTACACGACGCTCTTCCGAT |
| | CTGAGGTGAAGCTGGTGGAATC (SEQ ID NO: 112) |
| VHs10 | AATGATACGGCGACCACCGAGATCTACACTCTTTCCCTACACGACGCTCTTCCGAT |
| | CTGATGTGAACTTGGAAGTGTC (SEQ ID NO: 113) |
| VHs11 | AATGATACGGCGACCACCGAGATCTACACTCTTTCCCTACACGACGCTCTTCCGAT |
| | CTGAGGTCCAGCTGCAACAGTC (SEQ ID NO: 114) |
| VHs12 | AATGATACGGCGACCACCGAGATCTACACTCTTTCCCTACACGACGCTCTTCCGAT |
| | CTGAGGTGCAGCTGGAGGAGTC (SEQ ID NO: 115) |

TABLE 13
VL sens PCR_2 (v2) primer sequence

| | |
|---|---|
| VKs1 | AATGATACGGCGACCACCGAGATCTACACTCTTTCCCTACACGACGCTCTTCCGATC |
| | TGATATTGTGATGACGCAGGCT (SEQ ID NO: 116) |
| VKs2 | AATGATACGGCGACCACCGAGATCTACACTCTTTCCCTACACGACGCTCTTCCGATC |
| | TGATATTGTGATAACCCAG (SEQ ID NO: 117) |
| VKs3 | AATGATACGGCGACCACCGAGATCTACACTCTTTCCCTACACGACGCTCTTCCGATC |
| | TGACATTGTGCTGACCCAATCT (SEQ ID NO: 118) |
| VKs4 | AATGATACGGCGACCACCGAGATCTACACTCTTTCCCTACACGACGCTCTTCCGATC |
| | TGACATTGTGATGACCCAGTCT (SEQ ID NO: 119) |
| VKs5 | AATGATACGGCGACCACCGAGATCTACACTCTTTCCCTACACGACGCTCTTCCGATC |
| | TGATATTGTGCTAACTCAGTCT (SEQ ID NO: 120) |
| VKs6 | AATGATACGGCGACCACCGAGATCTACACTCTTTCCCTACACGACGCTCTTCCGATC |
| | TGATATCCAGATGACACAG ACT (SEQ ID NO: 121) |
| VKs7 | AATGATACGGCGACCACCGAGATCTACACTCTTTCCCTACACGACGCTCTTCCGATC |
| | TGACATCCAGCTGACTCAGTCT (SEQ ID NO: 122) |
| VKs8 | AATGATACGGCGACCACCGAGATCTACACTCTTTCCCTACACGACGCTCTTCCGATC |
| | TCAAATTGTTCTCACCCAGTCT (SEQ ID NO: 123) |
| VKs9 | AATGATACGGCGACCACCGAGATCTACACTCTTTCCCTACACGACGCTCTTCCGATC |
| | TGATGTTTTGATGACCCAAACT (SEQ ID NO: 124) |

The amplified and quality controlled VH and VL sequencing library was multiplexed (50% each) and sequenced on a MiSeq using 2*300 PE reads.

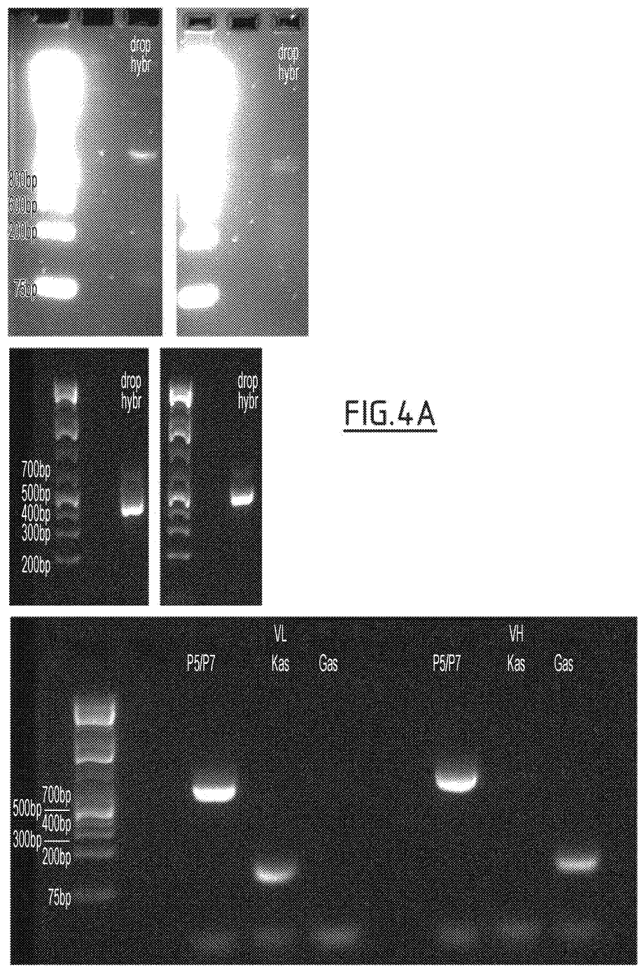

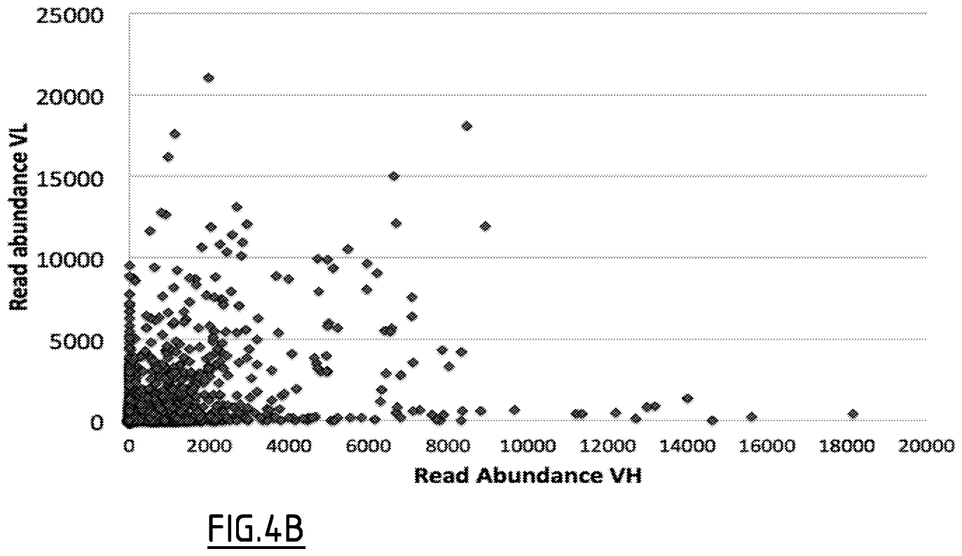

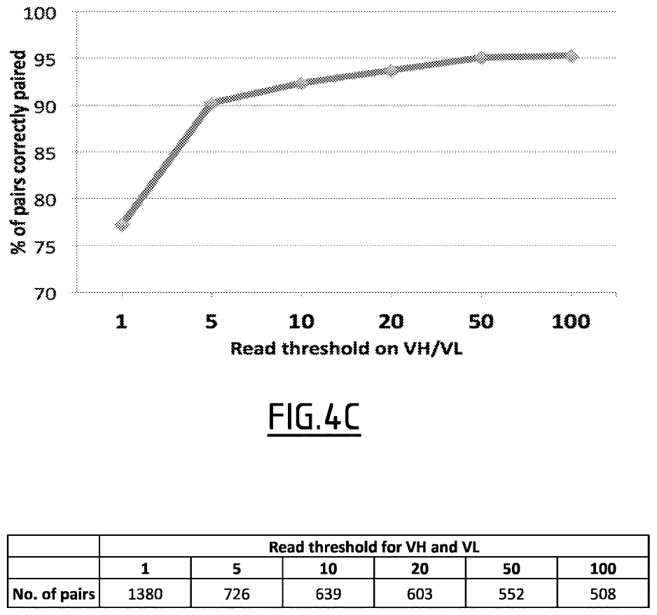

FIG. 4A depicts a nested PCR amplification of barcoded single cell VH/VL FIG. 4B depicts barcodes that were deconvoluted and plotted based on their respective number of reads.

FIG. 40 depicts paired sequence reads sharing the same barcode (coming from the same cells) that were plotted based on the number of VH and VL reads.

FIG. 4D depicts the correct hybridoma VH\NL pairing rate as a function of read threshold for each chain.

FIG. 4E depicts the number of hybridoma pairs pairing rate as a function of read threshold (for both chains).

### Example 5: Hybridoma Dilution

To assess VH\NL gene capture efficiency in a sub-nanoliter droplet, 3 mouse hybridoma cell line expressing known antibody sequences were mixed at a ratio of 60%-30%-10%. A total of 10,000 cells (plus 240,000 human non Ig expressing cells) were washed in PBS and resuspended in 97.4 μL of Cell Suspension Buffer (300 μL Percoll; 33.3 μL NaCl 1.5M; 10 μL Pluronic 10%; 25 μL Hepes 1M; 50 μL Serum low IgG 5%; 581.7 μL Media DMEM) plus 2.6 μL of 100 μM DY-647.

Cells were co-encapsulated with hydrogel beads library carrying the single cell barcodes and the primers for VH and VL mRNA capture and cDNA synthesis in the drop.

| | RT primer sequence: |
|---|---|
| | VH_1: |
| (SEQ ID NO: 9) | |
| | GGCCAGTGGATAGACAGATGGGGG |
| | VH_2: |
| (SEQ ID NO: 10) | |
| | GGCCAGTGGATAGACCGATGGGGC |
| | VH_3: |
| (SEQ ID NO: 11) | |
| | GGCCAGTGGATAGACTGATGGGGG |
| | VH_4: |
| (SEQ ID NO: 12) | |
| | GTCACCGCAGCCAGGGACCAAGGG |
| | RT_VLk: |
| (SEQ ID NO: 125) | |
| | GATGGTGGGAAGATGGATAC |

The hydrogel beads were washed 10 times in 1×BW buffer, spin at 2500 g for 2 min at 4° C. The double stranded barcodes were denatured in 1 mL denaturation solution (700 μL H₂O+300 μl 1M NaOH) at 22° C. for 2 min. The beads were washed 3 times in BW buffer, and labeled with 10 μL of FITC biotin 20 μM and incubate at room temperature on rotating platform for 10 min. Following 3 washes in BW buffer, the beads were heated for 2 min at 70° C. and supernatant were removed. The beads were washed with 200 μL lysis* buffer (80 μL 10× Lysis buffer {2% Triton, 30 mM MgCl₂, 500 mM Tris-HCl pH 7.4}, 8 μL Protease inhibitor, 8 μL DY-647, 4 μL DTT 1M). The beads were maintained in 50 μL lysis* buffer.

The RT mix was prepared as shown in Table 14.

TABLE 14

| | Volume |
|---|---|
| Mix | [μL] |
| 5x SuperScript III RT buffer | 52 |
| dNTP 10 mM | 13 |
| RNase inhibitor (20 U/μL) | 16.25 |
| SuperScript III RT (200 U/μL) | 16.25 |
| 100 μM DY-647 solution | 2.5 |

The Cells, Hydrogel beads and RT mix were co-encapsulated in 500 μL drops using the flow rates shown in Table 14 using the feedback module with Fluigent pumps.

TABLE 15

| Flow rates (μL/hr) | Reagent |
|---|---|
| 200-250 | 5% (w/w) surfactant in HFE-7500 |
| 74 (200 for a Syringe pump) | RT Mix |
| 74 (200 for a syringe pump) | Cells |
| 50 (100 for a syringe pump) | Hydrogel beads in lysis buffer |

RT reaction, emulsion breakage, aqueous phase containing hydrogel beads and cDNA were recovered/treated as described in Example 4.

Half of the recovered cDNA was purified two times in a row using RNAClean Beckman beads at 1× beads/aqueous phase volume. Eluted cDNA was used for VH\NL amplification by PCR (PCR primers listed in Example 4), as described in Tables 15A and 16B

TABLE 16A

| Mix | 1 tube | Final conc. | | N tubes |
|---|---|---|---|---|
| H₂O | 4.92 | — | | 4.92 |
| 5X Go Taq Buffer | 10 | 1x | | 10 |
| MgCl2 | 3 | 1.25 | mM | 3 |
| dNTP 10 mM | 1 | 0.2 | mM | 1 |
| Primer as - 5 μM | 2.5 | 0.25 | μM | 2.5 |
| Primer s - 1.5 μM | 8.33 | 0.25 | μM | 8.33 |
| Go Taq Polymerase | 0.25 | | | 0.25 |

| | | | |
|---|---|---|---|
| DNA template | 20 | — | |
| Total | 50 | — | 30 |

TABLE 16B
VH and VL
Thermo-cycling

| | | | Temp | | |
|---|---|---|---|---|---|
| | Time | | (°C) | | |
| Activate | 2 | min | 95 | | |
| Taq Polymerase: | | | | | |
| Denaturation: | 1 | min | 95 | | |
| Annealing: | 1 | min | 55 | {close oversize bracket} | 25 cycles |
| Extension: | 1 | min | 72 | | |
| Finishing: | 10 | min | 72 | | |

| | |
|---|---|
| Keep | 4 °C |

The VH and VL PCR1 products were purified two times using AMPure Beckman beads, at a 0.8× and a 1× ratio, respectively. Half of the PCR1 product was used for the PCR2 reaction, as shown in Tables 17A and 17B.

TABLE 17A

| Mix | 1 tube | Final conc. | | N tubes |
|---|---|---|---|---|
| H₂O | 9 | — | | 9 |
| 5X Go Taq Buffer | 10 | 1x | | 10 |
| MgCl2 | 3 | 1.25 | mM | 3 |
| dNTP 10 mM | 1 | 0.2 | mM | 1 |
| Primer as - 5 μM | 2.5 | 0.25 | μM | 2.5 |
| Primer s - 1.5 μM | 6.25 | 0.25 | μM | 6.25 |
| Go Taq Polymerase | 0.25 | | | 0.25 |

| | | | |
|---|---|---|---|
| DNA template | 18 | — | |
| Total | 50 | — | 32 |

TABLE 17B
VH and VL
Thermo-cycling

| | |
|---|---|
| | Temp |
| Time | (°C) |

| | | | | | |
|---|---|---|---|---|---|
| Activate | 2 | min | 95 | | |
| Taq Polymerase: | | | | | |
| Denaturation: | 1 | min | 95 | | |
| Annealing: | 1 | min | 55 | {close oversize bracket} | 15 cycles |
| Extension: | 1 | min | 72 | | |
| Finishing: | 10 | min | 72 | | |

| | |
|---|---|
| Keep | 4 °C |

Amplified products were size selected on agarose gel, gel purified, quality controlled and sequenced on MiSeq 2*300 PE reads.

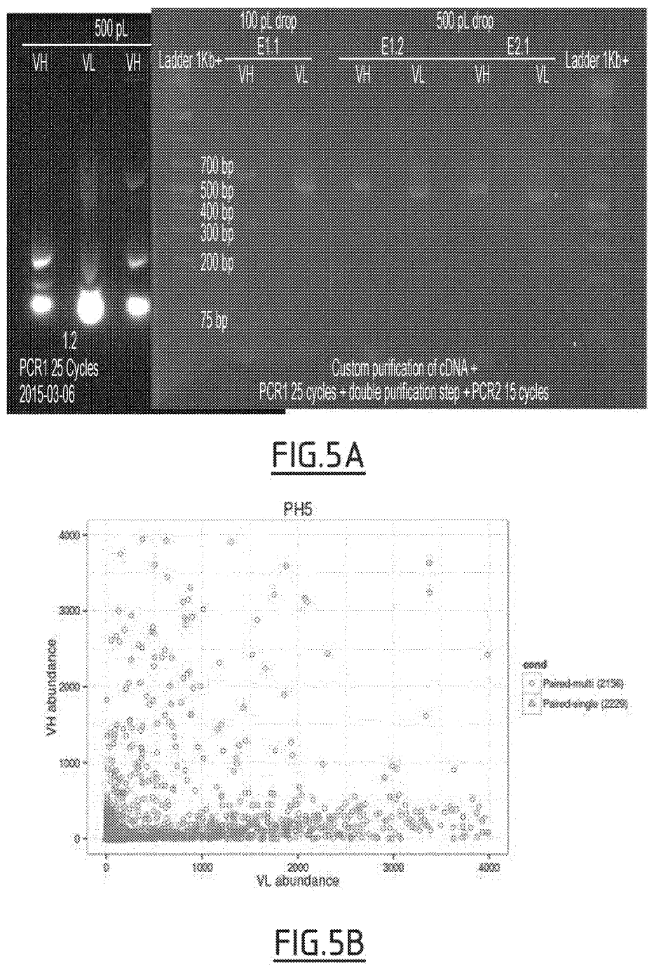

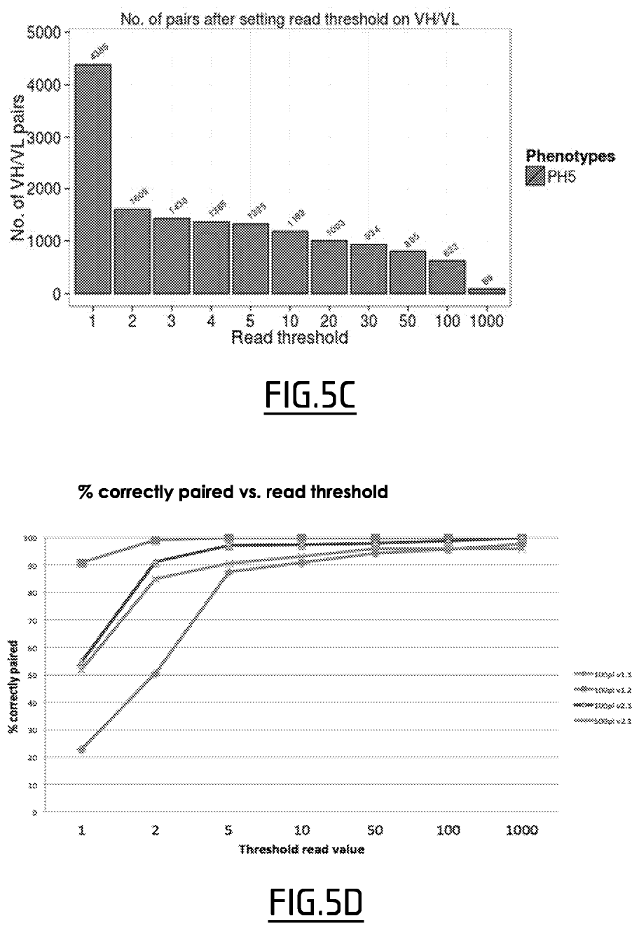

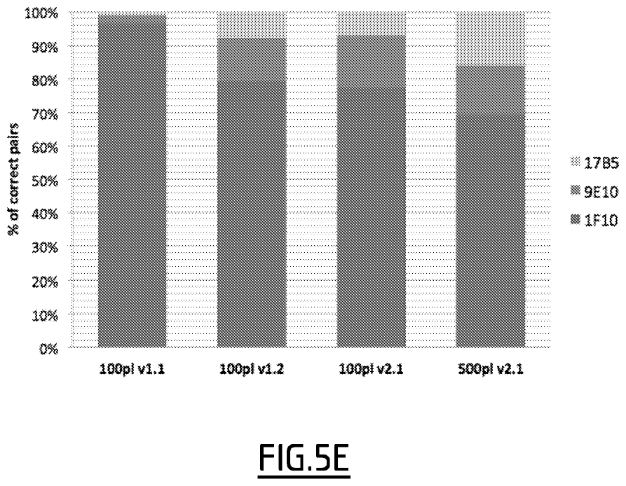

FIG. 5A depicts a nested PCR amplification of barcoded single cell VH\NL, depending on droplet volume (100 pL or 500 pL) and cDNA purification method (1 elution versus 2 elutions, named 1.1 or 1.2 and compared to 2 purifications, called 2.1).

FIG. 5B depicts paired sequence reads sharing the same barcode (coming from the same cells) that were plotted based on the number of VH and VL reads, in 500 pL drops and with improved cDNA purification (2 purifications).

FIG. 5C depicts the number of hybridoma pairs pairing rate as a function of read threshold (for both chains), in 500 pL drops and with improved cDNA purification (2 purifications).

FIG. 5D depicts the correct pairing rate of hybridoma pairs pairing rate as a function of read threshold (for both chains), droplet volume (100 pL or 500 pL) and cDNA purification method (1 elution versus 2 elutions, named 1.1 or 1.2 and compared to 2 purifications, called 2.1)

FIG. 5F depicts the recovered hybridoma pair distribution at a threshold of 10 reads per chain, depending on droplet volume (100 pL or 500 pL) and cDNA purification method (1 elution versus 2 elutions, named 1.1 or 1.2 and compared to 2 purifications, called 2.1). The 500 pL droplet volume with improved cDNA purification (2 purifications) leads to original 60-30-10% hybridoma ratio.

### Example 6: Screening Mouse Primary B Cells

Primary mouse spleen B cells secreting Tetanus Toxoid-specific antibodies were sorted and washed 2 times with 500 pL Cell Wash Buffer (915 pL DMEM/F12 cell culture media; 50 pL Serum Low IgG; 25 pL Hepes 1M; 10 pL Pluronic F68 10%). Finally, were resuspended in 97.4 pL CWB and 2.6 pL DY-647.

Single cells carrying hydrogel beads were prepared as in Example 5.

Co-encapsulation of cells, hydrogel beads and RT mix in 500 pL drops was performed as described in Example 5. Emulsion was broken as described in Example 5.

Aqueous phase containing hydrogel beads and cDNA were split into 2 equal volumes. Half of the aqueous phase was treated with 1 pL of RNAse A (for 15 min at 37° C.) and 4 pL of Proteinase K (over-night at 37° C.) and then half of the material was purified. Half of the aqueous phase was centrifuged at 10,000 g for 10 min at 4° C. Recovered supernatant was treated as above and then half of the material was purified. cDNA were purified twice with RNAClean Beads using 1× bead:aqueous phase volume ratio and used for the first VH/VL PCR amplification as shown in Table 18A and B.

TABLE 18A

| Mix | 1 tube | Final conc. | | N tubes |
|---|---|---|---|---|
| H₂O | 9 | — | | 9 |
| 5X Go Taq Buffer | 10 | 1x | | 10 |
| MgCl2 | 3 | 1.25 | mM | 3 |
| dNTP 10 mM | 1 | 0.2 | mM | 1 |
| Primer as - 5 μM | 2.5 | 0.25 | μM | 2.5 |
| Primer s - 1.5 μM | 6.25 | 0.25 | μM | 6.25 |
| Go Taq Polymerase | 0.25 | | | 0.25 |

| | | | |
|---|---|---|---|
| DNA template | 18 | — | |
| Total | 50 | — | 32 |

TABLE 18B
Thermo-cycling

| | | | Temp | | |
|---|---|---|---|---|---|
| | Time | | (°C) | | |
| Activate | 2 | min | 95 | | |
| Taq Polymerase: | | | | | |
| Denaturation: | 1 | min | 95 | | |
| Annealing: | 1 | min | 55 | {close oversize bracket} | 25 cycles |
| Extension: | 1 | min | 72 | | |
| Finishing: | 10 | min | 72 | | |

| | |
|---|---|
| Keep | 4 °C |

The amplified VH and VL products were purified twice at 0.8× and 1×AMPure beads ratio, respectively. Half of the purified material was used for the second nested PCR reaction, as shown in Table 18A and B.

TABLE 19A

| Mix | 1 tube | Final conc. | | N tubes |
|---|---|---|---|---|
| H₂O | 7 | — | | 49 |
| 5X Go Taq Buffer | 10 | 1x | | 70 |
| MgCl2 | 3 | 1.25 | mM | 21 |
| dNTP 10 mM | 1 | 0.2 | mM | 7 |
| Primer as - 5 μM | 2.5 | 0.25 | μM | 17.5 |
| Primer s - 1.5 μM | 6.25 | 0.25 | μM | 43.75 |
| Go Taq Polymerase | 0.25 | | | 1.75 |

| | | | |
|---|---|---|---|
| DNA template | 20 | — | |
| Total | 50 | — | 210 |

TABLE 19B
VH and VL
Thermo-cycling

| | | | Temp | | |
|---|---|---|---|---|---|
| | Time | | (°C) | | |
| Activate | 2 | min | 95 | | |
| Taq Polymerase: | | | | | |
| Denaturation: | 1 | min | 95 | | |
| Annealing: | 1 | min | 55 | {close oversize bracket} | 15 cycles |
| Extension: | 1 | min | 72 | | |
| Finishing: | 10 | min | 72 | | |

| | |
|---|---|
| Keep | 4 °C |

The VH/VL amplified products were then purified using PCR purification kit (QIAGEN). A tenth was used for the third PCR reaction, performed as shown in Table 19A and B.

TABLE 20A

| Mix | 1 tube | Final conc. | | N tubes |
|---|---|---|---|---|
| H₂O | 7 | — | | 78.05 |
| 5X Go Taq Buffer | 10 | 1x | | 28 |
| MgCl2 | 3 | 1.25 | mM | 8.4 |
| dNTP 10 mM | 1 | 0.2 | mM | 2.8 |
| Primer as - 5 μM | 2.5 | 0.25 | μM | 7 |
| Primer s - 1.5 μM | 6.25 | 0.25 | μM | 7 |
| Go Taq Polymerase | 0.25 | | | 1.75 |

| | | | |
|---|---|---|---|
| DNA template | 20 | — | |
| Total | 50 | — | 133 |

TABLE 20B
VH and VL
Thermo-cycling

| | | | Temp | | |
|---|---|---|---|---|---|
| | Time | | (°C) | | |
| Activate | 2 | min | 95 | | |
| Taq Polymerase: | | | | | |
| Denaturation: | 1 | min | 95 | | |
| Annealing: | 1 | min | 62 | {close oversize bracket} | 5 cycles |
| Extension: | 1 | min | 72 | | |
| Finishing: | 10 | min | 72 | | |

| | |
|---|---|
| Keep | 4 °C |

Amplified products were size selected on agarose gel, gel purified, quality controlled and sequenced on MiSeq 2*300 PE reads. Analysis was done by combining the information of the centrifuged and non-centrifuged cDNA sequencing library.

Sense VH and VL PCR primers used in the first and second PCR reaction were those described in the literature (Rohatgi, et al., 2008 Dec. 31; 339(2):205-19).

The Antisense primers used for PCR1 and 2 were as described in Example 4.

Sense primers used in
PCR3:
(SEQ ID NO: 126)
AATGATACGGCGACCACCGAGATCTACACTCTTTCCCTACACGACGCTCT
TCCGATCT.
Antisense primer used in PCR3:
(SEQ ID NO: 127)
CAAGCAGAAGACGGCATACGAGAT

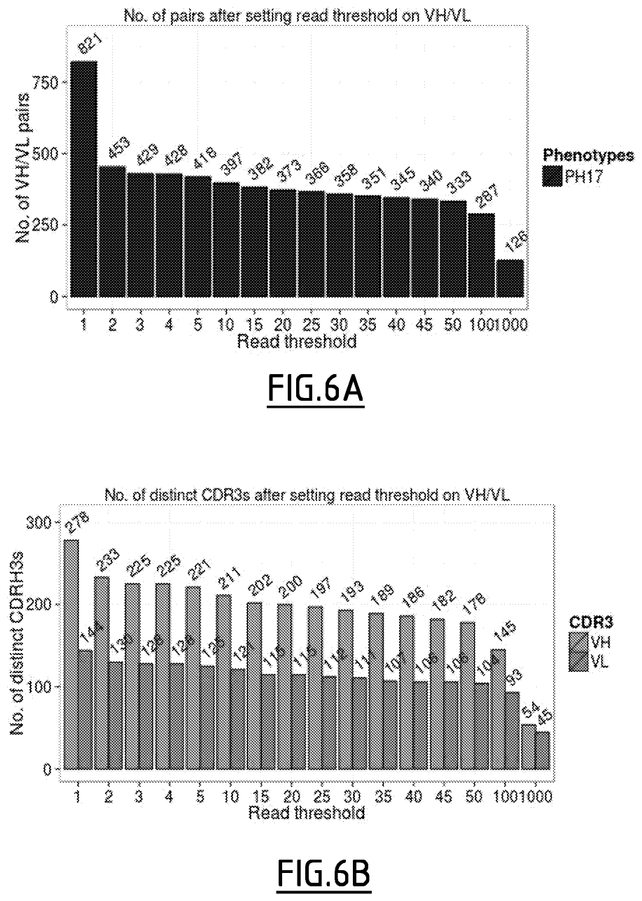

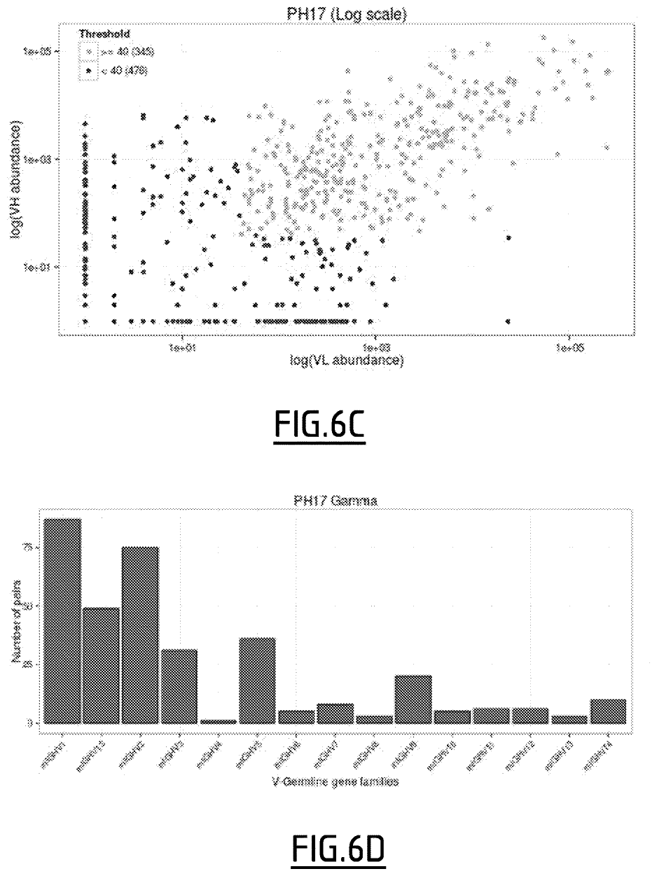

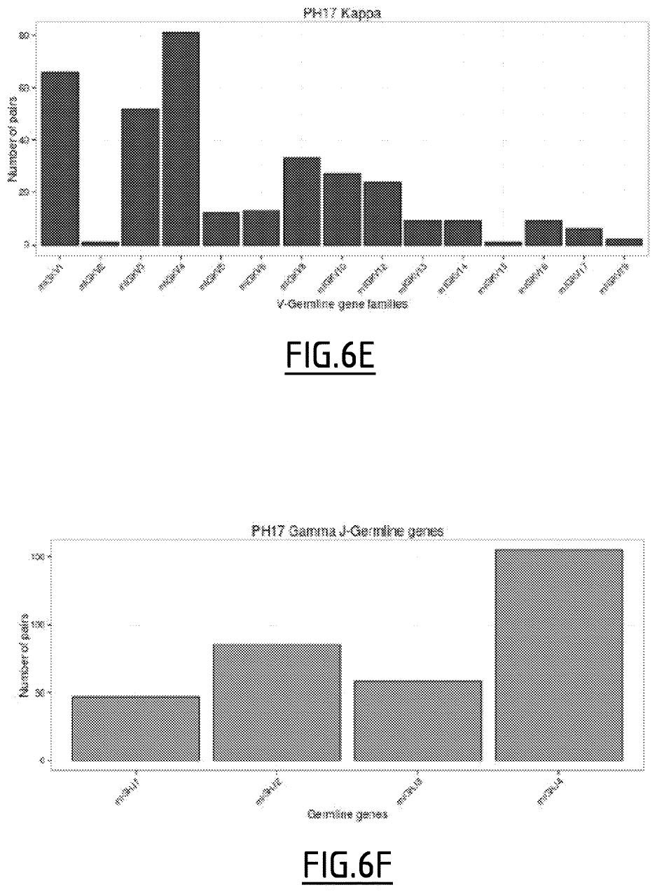

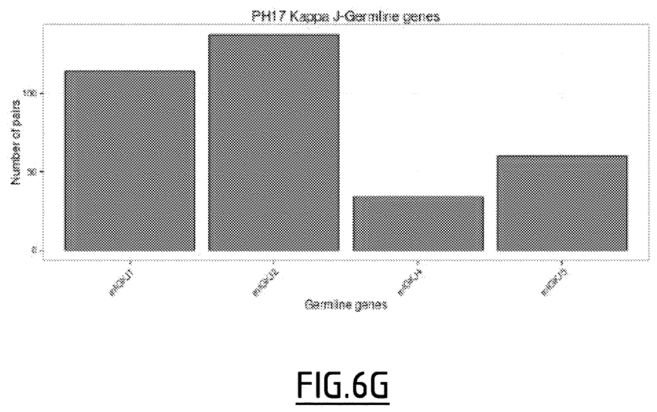

FIG. 6A depicts barcodes that were deconvoluted and plotted based on their respective number of reads.

FIG. 6B depicts the number of primary B cells VH\NL pairings as a function of read threshold (for both VH and VL chains).

FIG. 6C depicts the number of different complementarity determining regions (CDRs) for the heavy and light chains based on read threshold per chain.

FIG. 6D depicts paired sequences reads sharing the same barcode (coming from the same cells) were plotted based on the number of VH and VL reads.

FIG. 6E depicts heavy variable gene usage in the paired sequences (at a threshold of 40 reads per chain)

FIG. 6F depicts kappa variable gene usage in the paired sequences (at a threshold of 40 reads per chain)

FIG. 6G depicts heavy variable gene family usage in the paired sequences (at a threshold of 40 reads per chain)

FIG. 6H depicts heavy J gene family usage in the paired sequences (at a threshold of 40 reads per chain)

FIG. 6I depicts kappa J gene family

usage in the paired sequences (at a threshold of 40 reads per chain)

FIG. 6J depicts kappa variable gene family usage in the paired sequences (at a threshold of 40 reads per chain)

### Example 7: Assessing Impact of Gene Specific Primer Concentration

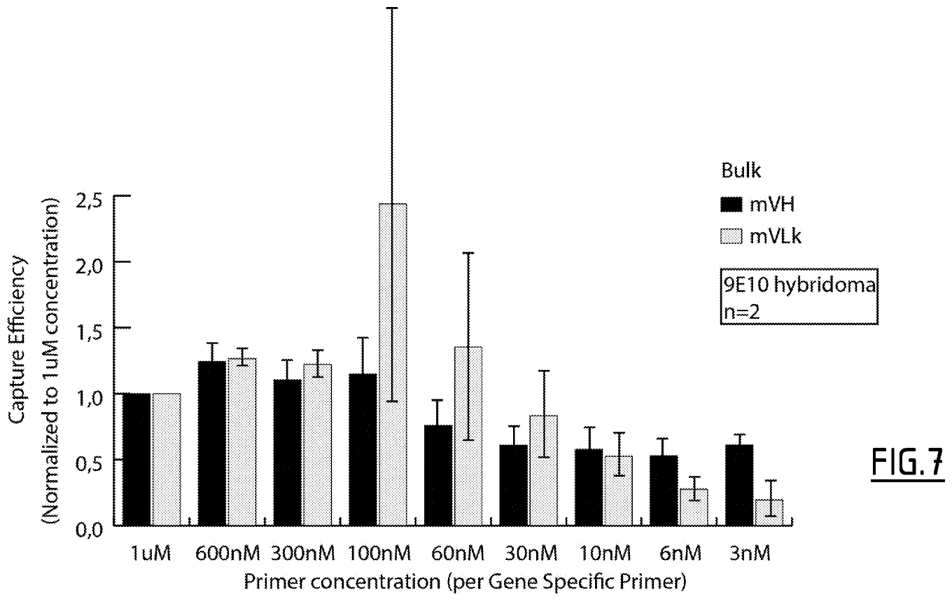

The impact of gene specific primer concentration in RT reaction has been assessed in bulk reactions for mouse antibody VH and VL starting from cells (see FIG. 7). It was aimed at mimicking the drop conditions (especially the volume of reaction buffer per cell, the amount of reagent per cell).

Therefore, 40 000 mouse 9E10 hybridoma cells were used (pre-washed in Cell wash buffer (DMEM F12 media, 0.1% Pluronic, 25 mM Hepes, 5% low IgG serum)) in reaction volume mimicking 500 pL per cell (20 uL final reaction). The mix was as indicated in table 21 below:

TABLE 21

| | Final | | |
|---|---|---|---|
| | concentration | 1 sample | |
| H₂O RNase/DNase free | | μL | |
| Cell Wash Buffer (Containing | | 5 | μL |
| 40 000 cells) | | | |

| | | | |
|---|---|---|---|
| Lysis Buffer 10x (2% Triton, | 1x | 2 | μL |
| 30 mM MgCl2, 500 mM | | | |
| Tris-HCl pH 7.4) | | | |
| 5x RT Buffer | 1x | 4 | μL |

| | | | | |
|---|---|---|---|---|
| dNTP 10 mM | 0.5 | mM | 1 | μL |
| 0.1M DTT | 5 | mM | 1 | μL |
| SS III RT enzyme | 12.5 | U/μl | 1.25 | μL |
| (200 U/μl) | | | | |
| RNase Inhibitor | 1.25 | U/μL | 1.25 | μL |

| | | | |
|---|---|---|---|
| Protease Inhibitor | 1x | 0.2 | μl |

| | | |
|---|---|---|
| (100x) | | |
| Final volume | 18 | μL |

Then different amount of VH and VL gene specific primer was added in concentrations as indicated in table 22.

TABLE 22

| | 1 uM | 600 nM | 300 nM | 100 nM | 60 nM | 30 nM | 10 nM | 6 nM | 3 nM |
|---|---|---|---|---|---|---|---|---|---|
| | each | each | each | each | each | each | each | each | each |
| Primers | 2 μL | 1.2 μL | 0.6 μL | 2 μL of | 1.2 μL | 0.6 μL | 2 μL of | 1.2 μL | 0.6 μL |
| | of | of | of | 1 uM | of | of | 0.1 uM | of | of |
| | 10 uM | 10 uM | 10 uM | each | 1 uM | 1 uM | each | 0.1 uM | 0.1 uM |
| | each | each | each | | each | each | | each | each |
| H2O | | 0.8 μL | 1.4 μL | | 0.8 μL | 1.4 μL | | 0.8 μL | 1.4 μL |

The pelleted cells were incubated into the mix for 10 min on ice to allow lysis to occur. The mix was incubated for 1 h at 55° C. mixed at 550 rpm. RT was then inactivated at 70° C. for 15 min at 550 rpm. Then cDNA was treated with RNAse A and PK to remove trace of PCR inhibitors. We added H₂O qsp 95 μL and added 1 μL of RNase A (10 mg/mL stock concentration, 100 ug/μL final concentration) and placed the DNase/RNase free DNA LoBind tube for 15 min at 37° C. We then added 4 μl of Proteinase K (20 mg/mL stock concentration, 0.8 mg/mL final concentration) and placed the tube for 1 h at 50° C. We inactivated PK for 15 minutes at 70° C. cDNA were then purified using RNACLeanup beads at 1× ratio (1 volume of bead solution with 1 volume of cDNA) and followed Beckman RNA Cleanup beads SOP. We eluted cDNA in 40 μL DNase/RNase free H₂O for 2 min and recovered eluted cDNA. We eluted for a second time the beads with 40 μL H₂O. The two elutions were pooled and we proceed to the second purification step using 1×RNAClean up beads (Beckman) exactly as before except only do a single elution with 40 μL H₂O. The amount of cDNA generated per condition was measured using absolute quantification qPCR based on multiplex qPCR reaction.

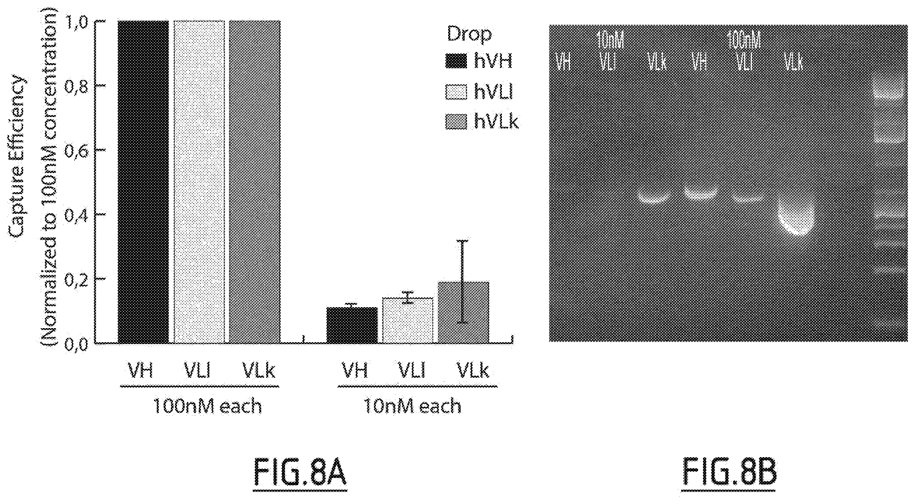

Impact of gene specific primer concentration was assessed in RT reaction in droplets for human antibody VH and VL starting from cell (see FIGS. 8A and B). 5000 human activated switched memory B cells from human healthy donor purified from PBMC and activated for 5 days were encapsulated in 500 μL drops containing either 100 nM or 10 nM of each gene specific primer for capturing heavy/light_kappa/Light_lambda chains. Each reagent (cells, lysis and RT reagents) was co-flowed in a microfluidic chip for 500 μL droplet production. The lysis/Pi/DTT/Dye containing solution was prepared as indicated below in table 23.

TABLE 23

| | | | Final | | Final | |
|---|---|---|---|---|---|---|
| | Volume | | Concentration in | | concentration | |
| Mix | [μl] | | the 200 μL mix | | in drop | |
| Lysis Buffer 10x (v2) | 80 | μL | 4x | | ≈1x | |
| (2% Triton, 30 mM | | | | | | |
| MgCl2, 500 mM | | | | | | |
| Tris-HCl pH 7.4) | | | | | | |
| 100x Protease Inhibitor | 8 | μL | 4x | | ≈1x | |
| Dye 647 100 uM | 8 | μL | 4 | μM | ≈1 | μM |
| 1M DTT | 4 | μL | 20 | mM | ≈5 | mM |

| | | | | |
|---|---|---|---|---|
| H₂O | 100 | μL | — | — |

50 μL of this mix was used for encapsulation and kept it on ice until aspiration into tip. The 2.6×RT mix preparation was prepared as indicated in table 24.

TABLE 24

| | | Final | | Final | |
|---|---|---|---|---|---|
| | Volume | Concentration | | concentration | |
| Mix | [μl] | in the mix | | in drop | |
| 5x SuperScript III RT | 52 | 1.25x | | 1x | |
| buffer | | | | | |
| dNTP 10 mM | 13 | 1.3 | mM | 0.5 | mM |
| RNase inhibitor (20 U/μL) | 16.25 | 3.25 | U/μL | 1.25 | U/μL |
| SuperScript III RT | 16.25 | 32.5 | U/μL | 12.5 | U/μL |
| (200 U/μl) | | | | | |
| Dye 647 100 μM | 2.5 | 2.5 | μM | 0.9375 | μM |

| | | | |
|---|---|---|---|
| Mix RT_primers 10 μM | 2.5 μL | 250 nM each | 100 nM each |
| each (or 1 uM each) | | GSP (or | GSP (or |
| | | 25 nM each) | 10 nM each) |

The cells washed and resuspended in Cell Suspension Buffer (CSB) containing the following reagents:

| | | |
|---|---|---|
| Pluronic F68 10% | 10 | μl |
| Hepes 1M | 25 | μl |
| Serum low IgG | 50 | μl |
| Media DMEM/F12 | 915 | μl |

The produced drops were collected on ice to allow lysis to occur. The emulsion was Incubated for 1 h at 55° C. mixed at 550 rpm. RT was then inactivated at 70° C. for 15 min at 550 rpm. Then cDNA was treated with RNAse A and PK to remove trace of PCR inhibitors.

We added H₂O qsp 95 μL and added 1 μL of RNase A (10 mg/mL stock concentration, 100 μg/μL final concentration) and placed the DNase/RNase free DNA LoBind tube for 15 min at 37° C. We then added 4 μl of Proteinase K (20 mg/mL stock concentration, 0.8 mg/mL final concentration) and placed the tube for 1 h at 50° C. We inactivated PK for 15 minutes at 70° C. cDNA was then purified using RNACLeanup beads at 0.8× ratio (1 volume of bead solution with 1 volume of cDNA) and followed Beckman RNA Cleanup beads SOP. We eluted cDNA in 40 μL DNase/RNase free H₂O for 2 min and recovered eluted cDNA.

The amount of cDNA generated per condition was measured using absolute quantification qPCR based on multiplex qPCR reaction.

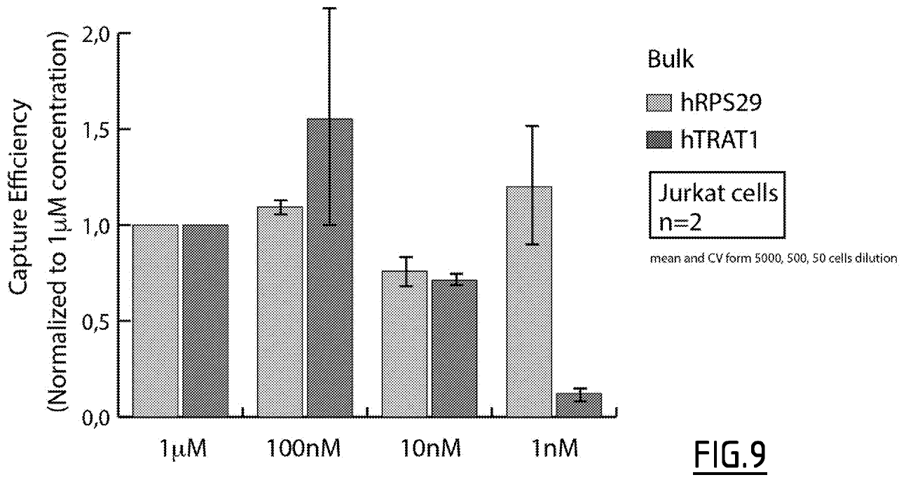

Impact of gene specific primer concentration in RT reaction was assessed in bulk reaction for non antibody gene starting from cells (see FIG. 9). We aimed at mimicking the drop conditions (especially the volume of reaction buffer per cell, the amount of reagent per cell).

We used 40 000 human Jurkat cells (pre-washed in Cell wash buffer (DMEM F12 media, 0.1% Pluronic, 25 mM Hepes, 5% low IgG serum)) in reaction volume mimicking 500 pL per cell (20 μL final reaction). The mix was as indicated in table 25:

TABLE 25

| | Final | | 1 μM | | 0.1 μM | | 0.01 μM | | 0.001 μM | |
|---|---|---|---|---|---|---|---|---|---|---|
| Mix (500 pl) | conc | | oligo | | oligo | | oligo | | oligo | |
| H₂O RNAse/DNase free | | | 2.3 | μL | 4.1 | μL | 2.3 | μL | 4.1 | μL |
| Cell wash buffer | | | 5 | μL | 5 | μL | 5 | μL | 5 | μL |
| (containing 40.000 cells) | | | | | | | | | | |
| Lysis Buffer 10x (v2) | 1X | | 2 | μL | 2 | μL | 2 | μL | 2 | μL |
| (2% Triton, 30 mM | | | | | | | | | | |
| MgCl₂, 500 mM | | | | | | | | | | |
| Tris-HCl pH 7.4) | | | | | | | | | | |
| 5xRT buffer | 1X | | 4 | μL | 4 | μL | 4 | μL | 4 | μL |
| dNTP 10 mM | 0.5 | mM | 1 | μL | 1 | μL | 1 | μL | 1 | μL |
| 0.1M DTT | 5 | mM | 1 | μL | 1 | μL | 1 | μL | 1 | μL |
| SS III RT enzyme | 12.5 | U/μL | 1.25 | μL | 1.25 | μL | 1.25 | μL | 1.25 | μL |
| (200 U/μL | | | | | | | | | | |
| RNase inhibitor | 12.5 | U/μL | 1.25 | μL | 1.25 | μL | 1.25 | μL | 1.25 | μL |

| | | | | | | | | | |
|---|---|---|---|---|---|---|---|---|---|
| 100x Protease Inhibitor | 1x | 0.2 | μL | 0.2 | μL | 0.2 | μL | 0.2 | μL |

| RT TRAT1/RPS29 primers | 2 μL | | 2 μL | 2 μL | 2 μL |
|---|---|---|---|---|---|
| | (of 10 μM | | (of 10 μM | (of 0.1 μM | (of 0.1 μM |
| | each) | | each) | each) | each) |
| Final volume | 20 | μL | — | | |

The pelleted cells were incubated into the mix for 10 min on ice to allow lysis to occur. The mix was Incubated for 1 h at 55° C. mixed at 550 rpm. RT was then inactivated at 70° C. for 15 min at 550 rpm. Then cDNA was treated with RNAseq A and PK to remove trace of PCR inhibitors. We added H₂O qsp 95 uLμL and added 1 μl of RNase A (10 mg/mL stock concentration, 100 ug/uLμμ final concentration) and placed the DNase/RNase free DNA LoBind tube for 15 min at 37° C. We then added 4 μl of Proteinase K (20 mg/mL stock concentration, 0.8 mg/mL final concentration) and placed the tube for 1 h at 50° C. We inactivated PK for 15 minutes at 70° C. cDNa were then purified using RNACLeanup beads at 1× ratio (1 volume of bead solution with 1 volume of cDNA) and followed Beckman RNA Cleanup beads SOP. We eluted cDNA in 40 μL DNase/RNase free H₂O for 2 min and recovered eluted cDNA. We eluted for a second time the beads with 40 μL H₂O. The two elutions were pooled together. The amount of cDNA generated per condition was measured using relative quantification qPCR based on simplex qPCR reaction.

### Example 8: Assessing Impact of polydT Primer Concentration in RT Reaction

Assessing impact of polydT primer concentration in RT reaction for human whole transcriptome capture starting from cells has been performed in droplet reaction. A mixture of 20,000 cells composed of 95% human B cell Ramos cells and 5% human T cell Leukemia Jurkat cells was encapsulated in 500 μl droplets with Lysis buffer, RT reagents and either 3.3 uM or 100 nM or 33 nM of polydT primer. All reagents were co-flowed in a microfluidic chip for 500 μl droplets production.

The mix containing RT reagents was prepared as indicated in table 26 below.

TABLE 26

| | | | Volume | Final | |
|---|---|---|---|---|---|
| | Concentration | | (μL) | concentration | Units |
| First Strand buffer | 5X | | 23.96 | 0.46 | X |
| IGEPAL CA-630 | 10% (V/V) | | 9.16 | 0.35 | % |
| dNTPs | 10 | mM | 12.21 | 0.47 | mM |
| DTT | 0.1M | | 8.14 | 0 | M |
| Tris-Hcl | 1M | | 12.21 | 0.05 | M |
| Murine Rnase | 40 | U/μL | 8.14 | 1.24 | U/μL |
| inhibitor | | | | | |
| Superscript III RT | 200 | U/μL | 12.21 | 9.3 | U/μL |
| enzyme | | | | | |
| Optiprep | 100x | | 3.53 | | |
| Nuclease Free water | | | 6.19 | | |
| Dye 647 | 100 | μM | 2 | 2 | uM |
| Total | | | 97.75 | | |

Then polydT RT primer were added at different concentration as indicated in table 27.

TABLE 27

| | | | | | | | Number |
|---|---|---|---|---|---|---|---|
| | | | Volume | Volume | Final | | of primer/ |
| | Concentration | | (μL) | water | Concentration | | droplet |
| polydT primer | 100 | μM | 0.748 | 1.5 | 3.3 | μM | 10\hat{ }9 |
| polydT primer | 10 | μM | 2.25 | — | 0.10 | μM | 3.10\hat{ }7 |
| polydT primer | 10 | μM | 0.748 | 1.5 | 0.033 | μM | 10\hat{ }7 |

Cells were resuspended in a mix containing the concentrations indicated in table 28.

TABLE 28

| | | | Volume | Final | |
|---|---|---|---|---|---|
| | Concentration | | (μL) | Concentration | Units |
| Cellules en PBS 1X | 1X | | 50 | 0.23255814 | X |
| Optiprep | 100x | | 16 | 7.441860465 | X |
| BSA | 0.2 | mg/mL | 4 | 0.0037 | mg/ml |
| Dye 647 | 100 | μM | 2 | 2 | uM |
| Nuclease free water | | | 28 | | |
| Volume total | | | 100 | | |

The last mix is composed of the ingredients and concentrations as indicated in table 29.

TABLE 29

| | | | Volume | Final | |
|---|---|---|---|---|---|
| | Concentration | | (μL) | concentration | Units |
| 10 mM Tris HCl | 2x | | 7.33 | | |
| (pH 8), 0.1 mM | | | | | |
| EDTA and 0.1% | | | | | |
| (v/v) Tween 20 | | | | | |
| BSA | 0.2 | mg/mL | 0.345 | 0.0037 | mg/ml |
| Nuclease free water | — | | 40.325 | | |
| Dye 647 | 100 | μM | 2 | 2 | uM |
| Volume total | | | 50 | | |

The droplets produced were collected on ice to allow Lysis to occur. The emulsion was incubated 2 h at 50° C. and RT was then inactivated at 70° C. for 15 min. The emulsion was then cooled down on ice and droplets were broken by adding 1 volume of a solution composed of 80% (v/v) HFE-7500 and 20% (v/v) perfluorooctanol. Then cDNA was centrifuged 15 min at 14,000 g at 4° C. and the supernatant recovered. To digest excess of primers and primer-dimers, cDNA were incubated with 1 μL of Exonuclease I (NEB, reference #M0293; 20,000units/ml), 1 μL of Hinf1 (NEB, #R0155; 10,000units/ml) for 30 min at 37° C. We inactivated Exonuclease I and Hinf1 enzymes for 10 min at 80° C. cDNA were then purified using AgencourtAMPure beads at 1,2× ratio (1,2 volume of bead with 1 volume cDNA) following Beckman Agencourt AMPure SOP. cDNA were eluted in 17 μL DNAse/RNAse free water for 2 min and we recovered eluted cDNA. The amount of cDNA generated per condition was measured using relative quantification qPCR based on simplex qPCR reaction.

### Example 9: Impact of Gene Specific Primer Concentration on Gene Specific Reverse Transcription

Capture efficiency (normalized to the 1 uM each gene specific primer) of mouse immonuglobulin variable heavy (VH) and variable light (VL) chains reverse transcription. 9E10 hybridoma cells were processed in bulk in RT reaction mimicking droplet conditions. This experiment mimics a drop condition (especially the volume of reaction buffer per cell, the amount of reagent per cell). For each condition, we used from 1 uM to 10 nM of each VH and VL gene specific primer. Comparisons are based on qPCR data. The Cp value (crossing point value determined by the 2^{nd} derivative max calculation method) was compared to each primer conditions. (n=2). Error bar=standard deviation (FIG. 7).

Capture efficiency (normalized to the 100 nM each gene specific primer) of human immonuglobulin variable heavy (VH) and variable light kappa (VLk) and lambda (VLI) chains reverse transcription. Human switched activated memory B cells (day 5 post activation) were encapsulated in 500 μL drops with lysis and RT reagents and either 100 nM or 10 nM of each gene specific primer was added in the mix. (Left) Comparisons are based on qPCR data for each immunoglobulin variable chain. The Cp value (crossing point value determined by the 2^{nd} derivative max calculation method) was compared to each primer conditions. (Right) Two steps nested PCR reaction amplified each VH and VL amplicon which were loaded on a 2% agarose gel stained with Sybr Gold. (n=2). Error bar=standard deviation (FIG. 8 A and B).

Capture efficiency (normalized to the 1 uM each gene specific primer) of human house keeping gene RPS29 and TRAT1 (from high to low expressed gene). Human T cell Leukemia (Jurkat cells) were processed in bulk in RT reaction mimicking droplet conditions and with either 1 uM, 100 nM, 10 nM, 1 nM of each gene specific primer. Comparisons are based on qPCR data for each specific gene. (n=2, 3 cDNA dilutions for each qPCR). Error bar=standard deviation. The highest expressed gene is less affected by primer concentration whereas the capture efficiency and RT of the lowest expressed gene is affected at 10 nM concentration of its RT primer concentration (FIG. 9).

### Example 10: Impact of polydT Primer Concentration on Whole Transcriptome Capture and Reverse Transcription

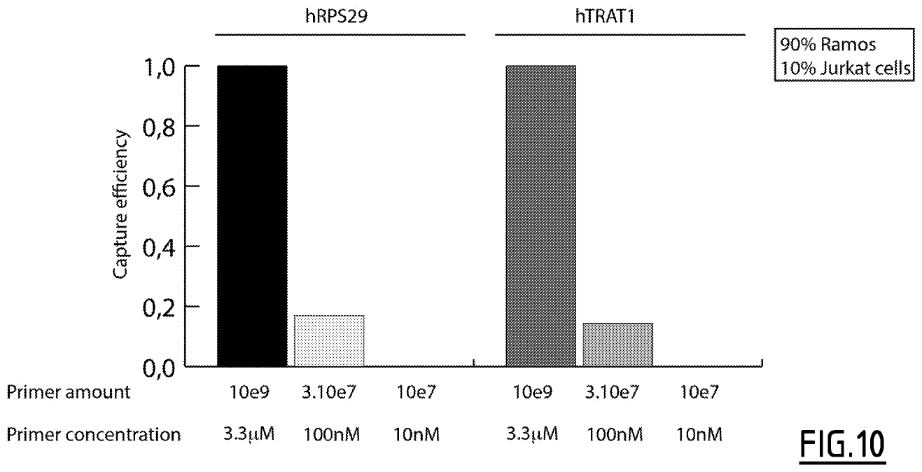

Capture efficiency of human housekeeping genes RPS29 and TRAT1 (from high to low expressed gene). Human T cell Leukemia (Jurkat) cells were encapsulated in 500 μL drops with lysis and RT reagents and either 3.3 μM or 100 or 33 nM of polydT primer was added in the mix. Comparisons are based on qPCR data for each specific gene. (n=1). n.d.=not detected. Both the highest and the lowest expressed gene are affected by primer concentration especially in low polydT primer concentration (FIG. 10).

1. A method for capturing and barcoding single cell nucleic acids comprising:
a) providing a plurality of cells contained within a plurality of compartments, at least some of the compartments comprising a single cell, a reverse transcriptase, and an oligonucleotide comprising a barcode sequence, and wherein the compartments of the plurality of compartments contain one or more barcode sequences distinguishable from barcode sequences contained in other compartments of the plurality of compartments;
b) lysing at least some of the cells within the compartments to release nucleic acids from the cells;
c) hybridizing at least some of the released nucleic acids to said oligonucleotide in at least some of the compartments;
d) reverse transcribing the released nucleic acids hybridized to said oligonucleotide in at least some of the compartments;
wherein each of said at least some of the compartments comprises the reverse transcriptase at a concentration of from about 1 U/μL to about 50 U/μL; and
wherein each of said at least some of the compartments has a volume of from about 0.1 nL to about 3 nL.

2. The method according to claim 1, wherein the concentration of the oligonucleotide in the compartments is at least 100 nM.

3. The method according to claim 1, wherein at least some of the compartments further comprise a particle.

4. The method according to claim 3, wherein oligonucleotide is bound to said particle.

5. The method according to claim 1, wherein the plurality of compartments has a volume equal to or less than 1 nL.

6. The method according to claim 1, which further comprises recovering single cell cDNAs produced by reverse transcription in at least some of the compartments.

7. The method according to claim 1, wherein the one or more barcode sequences uniquely identify nucleic acids released by a single cell from nucleic acids released from other cells.

8. The method according to claim 1, wherein the oligonucleotide further comprises a primer sequence selected from the group consisting of a poly-T sequence, a random DNA sequence, and a gene-specific sequence.

9-12. (canceled)

13. The method according to claim 1, wherein the plurality of compartments is a plurality of droplets.

14. A plurality of compartments wherein at least some of the compartments comprise (i) a single cell or a single cell lysate comprising nucleic acids, (ii) an oligonucleotide, and (iii) a reverse transcriptase at a concentration of from about 1 U/μL to about 50 U/μL, wherein the oligonucleotide comprises a barcode sequence, and wherein the compartments of the plurality of the compartments contain one or more barcode sequences distinguishable from barcode sequences contained in other compartments of the plurality of compartments;
wherein the plurality of compartments has a volume of less than 3 nL.

15. The plurality of compartments according to claim 14, wherein the concentration of the oligonucleotide in the compartments is at least 100 nM.

16. The plurality of compartments according to claim 14, wherein the at least some of the compartments further comprise (iv) a particle.

17. The plurality of compartments according to claim 14, wherein the plurality of compartments have a volume equal to or less than 1 nL.

18. The plurality of compartments according to claim 14, wherein the plurality of compartments is a plurality of droplets.

19. A method for preparing a plurality of microfluidic droplets comprising the following steps:
providing a first fluid source, the first fluid comprising a suspension of cells,
providing a second fluid source, the second fluid comprising an oligonucleotide, comprising a barcode sequence, wherein the droplets of the plurality of the microfluidic droplets contain one or more barcode sequences distinguishable from barcode sequences contained in other droplets of the plurality of microfluidic droplets;
providing a carrier fluid, the carrier fluid being immiscible with the first fluid and the second fluid;
injecting the carrier fluid in a main channel of a chip;
generating a flow of droplets in the carrier fluid by injecting the second fluid and the first fluid in at least a secondary channel of the chip, the secondary channel opening in the main channel, each generated droplet comprising a mix of the first fluid and the second fluid,
wherein the concentration of the cells in the first fluid, the concentration of oligonucleotides in the second fluid, the geometry of the main channel and the secondary channel, the injection parameters of the first fluid, of the second fluid and of the carrier fluid are adapted such that one or more of the droplets of the plurality of microfluidic droplets comprise a single cell and a volume of less than 3 nL.

20. (canceled)

21. The method according to claim 1, wherein the reverse transcriptase is at a concentration of from about 5 U/μL to about 50 U/μL or about 5 U/μL to about 25 U/μL.

22. The method according to claim 1, wherein each of said at least some of the compartments has a volume of from about 0.1 nL to about 2 nL.

23. The method according to claim 1, wherein each of said at least some of the compartments has a volume equal to or less than 1 nL.

24. The method according to claim 1, wherein the at least some of the compartments have an efficiency of reverse transcription of at least 10%.

25. The method according to claim 24, wherein the efficiency of reverse transcription in the at least some of the compartments is relative to that of purified RNA extracted from the cells.


SEQUENCE LISTING (139 sequences)

SEQ ID NO: 1 - DNA, 58 bp, synthetic construct
  misc_feature 1..58: note = antisense primer Top_SBS12-ATP5G3
  source 1..58: mol_type = other DNA
caacgtgactggagttcagacgtgtgctcttccgatctctgcttcagcgaagggtttc

SEQ ID NO: 2 - DNA, 57 bp, synthetic construct
  misc_feature 1..57: note = antisense primer Top_SBS12- RPS29
  source 1..57: mol_type = other DNA
caacgtgactggagttcagacgtgtgctcttccgatctacagacacgacaagagcga

SEQ ID NO: 3 - DNA, 59 bp, synthetic construct
  misc_feature 1..59: note = antisense primer Top_SBS12- ZNF780A
  source 1..59: mol_type = other DNA
caacgtgactggagttcagacgtgtgctcttccgatcttgatccatggaccatgttgct

SEQ ID NO: 4 - DNA, 53 bp, synthetic construct
  misc_feature 1..53: note = sense primer SBS3-ATP5G3
  source 1..53: mol_type = other DNA
acactctttccctacacgacgctcttccgatctcaggtgctgcaacagtagga

SEQ ID NO: 5 - DNA, 53 bp, synthetic construct
  misc_feature 1..53: note = sense primer SBS3-RPS29
  source 1..53: mol_type = other DNA
acactctttccctacacgacgctcttccgatctttacctcgttgcactgctga

SEQ ID NO: 6 - DNA, 52 bp, synthetic construct
  misc_feature 1..52: note = sense primer SBS3-ZNF780A
  source 1..52: mol_type = other DNA
acactctttccctacacgacgctcttccgatctagagcgttactgctgcaca

SEQ ID NO: 7 - DNA, 24 bp, synthetic construct
  misc_feature 1..24: note = antisense primer for mouse ATP5G3
  source 1..24: mol_type = other DNA
caactgcaactctggatccagctc

SEQ ID NO: 8 - DNA, 53 bp, synthetic construct
  misc_feature 1..53: note = sense primer for mouse ATP5G3
  source 1..53: mol_type = other DNA
acactctttccctacacgacgctcttccgatcttcgcctgtcacctagatcca

SEQ ID NO: 9 - DNA, 24 bp, synthetic construct
  misc_feature 1..24: note = RT primer VH_1 for VH
  source 1..24: mol_type = other DNA
ggccagtggatagacagatggggg

SEQ ID NO: 10 - DNA, 24 bp, synthetic construct
  misc_feature 1..24: note = RT primer VH_2 for VH
  source 1..24: mol_type = other DNA
ggccagtggatagaccgatggggc

SEQ ID NO: 11 - DNA, 24 bp, synthetic construct
  misc_feature 1..24: note = RT primer VH_3 for VH
  source 1..24: mol_type = other DNA
ggccagtggatagactgatggggg

SEQ ID NO: 12 - DNA, 24 bp, synthetic construct
  misc_feature 1..24: note = RT primer VH_4 for VH
  source 1..24: mol_type = other DNA
gtcaccgcagccagggaccaaggg

SEQ ID NO: 13 - DNA, 20 bp, synthetic construct
  misc_feature 1..20: note = RT primer VLk_1 for VH
  source 1..20: mol_type = other DNA
gcgtttcatttccagcttgg

SEQ ID NO: 14 - DNA, 20 bp, synthetic construct
  misc_feature 1..20: note = RT primer VLk_2 for VH
  source 1..20: mol_type = other DNA
gcgtttgatttccagcttgg

SEQ ID NO: 15 - DNA, 20 bp, synthetic construct
  misc_feature 1..20: note = RT primer VLk_3 for VH
  source 1..20: mol_type = other DNA
gcgttttatttccaattttg

SEQ ID NO: 16 - DNA, 28 bp, synthetic construct
  misc_feature 1..28: note = T7 antisense PCR1 primer
  source 1..28: mol_type = other DNA
gaatttaatacgactcactatagggaga

SEQ ID NO: 17 - DNA, 20 bp, synthetic construct
  misc_feature 1..20: note = sense primer MmLH_AG1
  source 1..20: mol_type = other DNA
taactgcaggtgtccactcc

SEQ ID NO: 18 - DNA, 20 bp, synthetic construct
  misc_feature 1..20: note = sense primer MmLH_AG2
  source 1..20: mol_type = other DNA
cagctacaggtgtccactcc

SEQ ID NO: 19 - DNA, 20 bp, synthetic construct
  misc_feature 1..20: note = primer MmLH_AG3
  source 1..20: mol_type = other DNA
tttatcaaggtgtgcattgt

SEQ ID NO: 20 - DNA, 20 bp, synthetic construct
  misc_feature 1..20: note = primer MmLH_AG4
  source 1..20: mol_type = other DNA
gaactgcaggcgtccactct

SEQ ID NO: 21 - DNA, 20 bp, synthetic construct
  misc_feature 1..20: note = sense primer MmLH_AG5
  source 1..20: mol_type = other DNA
taactgcaggtgttcactcc

SEQ ID NO: 22 - DNA, 20 bp, synthetic construct
  misc_feature 1..20: note = sense primer MmLH_AG6
  source 1..20: mol_type = other DNA
tcccaagctgtgtcctatcc

SEQ ID NO: 23 - DNA, 20 bp, synthetic construct
  misc_feature 1..20: note = sense primer MmLH_AG7
  source 1..20: mol_type = other DNA
ttccaagctgtgtcctgtcc

SEQ ID NO: 24 - DNA, 20 bp, synthetic construct
  misc_feature 1..20: note = sense primer MmLH_AG8
  source 1..20: mol_type = other DNA
cttttaaaggtattcactgt

SEQ ID NO: 25 - DNA, 20 bp, synthetic construct
  misc_feature 1..20: note = sense primer MmLH_AG9
  source 1..20: mol_type = other DNA
ttttaaaaggggtccagtgt

SEQ ID NO: 26 - DNA, 20 bp, synthetic construct
  misc_feature 1..20: note = sense primer MmLH_AG10
  source 1..20: mol_type = other DNA
ttttaaaaggtgtccagtgt

SEQ ID NO: 27 - DNA, 20 bp, synthetic construct
  misc_feature 1..20: note = sense primer MmLH_AG11
  source 1..20: mol_type = other DNA
ttttaaatggtatccagtgt

SEQ ID NO: 28 - DNA, 20 bp, synthetic construct
  misc_feature 1..20: note = sense primer MmLH_AG12
  source 1..20: mol_type = other DNA
ctgcccaaagtgcccaagca

SEQ ID NO: 29 - DNA, 20 bp, synthetic construct
  misc_feature 1..20: note = sense primer MmLH_AG13
  source 1..20: mol_type = other DNA
ctgcccaaagtatccaagca

SEQ ID NO: 30 - DNA, 25 bp, synthetic construct
  misc_feature 1..25: note = sense primer MmLHa
  source 1..25: mol_type = other DNA
atgrasttskggytmarctkgrttt

SEQ ID NO: 31 - DNA, 26 bp, synthetic construct
  misc_feature 1..26: note = sense primer MmLHb
  source 1..26: mol_type = other DNA
atgraatgsasctgggtywtyctctt

SEQ ID NO: 32 - DNA, 29 bp, synthetic construct
  misc_feature 1..29: note = sense primer MmLHc1
  source 1..29: mol_type = other DNA
atggactccaggctcaatttagttttcct

SEQ ID NO: 33 - DNA, 26 bp, synthetic construct
  misc_feature 1..26: note = sense primer MmLHc2
  source 1..26: mol_type = other DNA
atggctgtcytrgbgctgytcytctg

SEQ ID NO: 34 - DNA, 29 bp, synthetic construct
  misc_feature 1..29: note = sense primer MmLHc3
  source 1..29: mol_type = other DNA
atggvttggstgtggamcttgcyattcct

SEQ ID NO: 35 - DNA, 26 bp, synthetic construct
  misc_feature 1..26: note = sense primer MmLHd1
  source 1..26: mol_type = other DNA
atgaaatgcagctggrtyatsttctt

SEQ ID NO: 36 - DNA, 26 bp, synthetic construct
  misc_feature 1..26: note = sense primer MmLHd2
  source 1..26: mol_type = other DNA
atggrcagrcttacwtyytcattcct

SEQ ID NO: 37 - DNA, 26 bp, synthetic construct
  misc_feature 1..26: note = sense primer MmLHd3
  source 1..26: mol_type = other DNA
atgatggtgttaagtcttctgtacct

SEQ ID NO: 38 - DNA, 26 bp, synthetic construct
  misc_feature 1..26: note = sense primer MmLHe1
  source 1..26: mol_type = other DNA
atgggatggagctrtatcatsytctt

SEQ ID NO: 39 - DNA, 23 bp, synthetic construct
  misc_feature 1..23: note = sense primer mmlhe2
  source 1..23: mol_type = other DNA
atgaagwtgtggbtraactggrt

SEQ ID NO: 40 - DNA, 25 bp, synthetic construct
  misc_feature 1..25: note = sense primer MmLHe3
  misc_feature 15: note = n= I
  source 1..25: mol_type = other DNA
atggratggasckknrtctttmtct

SEQ ID NO: 41 - DNA, 25 bp, synthetic construct
  misc_feature 1..25: note = sense primer MmLHf1
  source 1..25: mol_type = other DNA
atgaacttygggytsagmttgrttt

SEQ ID NO: 42 - DNA, 25 bp, synthetic construct
  misc_feature 1..25: note = sense primer MmLHf2
  source 1..25: mol_type = other DNA
atgtacttgggactgagctgtgtat

SEQ ID NO: 43 - DNA, 23 bp, synthetic construct
  misc_feature 1..23: note = sense primer MmLHf3
  source 1..23: mol_type = other DNA
atgagagtgctgattcttttgtg

SEQ ID NO: 44 - DNA, 28 bp, synthetic construct
  misc_feature 1..28: note = sense primer MmLHf4
  source 1..28: mol_type = other DNA
atggattttgggctgattttttttattg

SEQ ID NO: 45 - DNA, 24 bp, synthetic construct
  misc_feature 1..24: note = VL sense primer MmLKa
  source 1..24: mol_type = other DNA
atgragwcacakwcycaggtcttt

SEQ ID NO: 46 - DNA, 25 bp, synthetic construct
  misc_feature 1..25: note = VL sense primer MmLKb
  source 1..25: mol_type = other DNA
atggagacagacacactcctgctat

SEQ ID NO: 47 - DNA, 29 bp, synthetic construct
  misc_feature 1..29: note = VL sense primer MmLKc
  source 1..29: mol_type = other DNA
atggagwcagacacactsctgytatgggt

SEQ ID NO: 48 - DNA, 32 bp, synthetic construct
  misc_feature 1..32: note = VL sense primer MmLKd1
  misc_feature 27: note = n=I
  source 1..32: mol_type = other DNA
atgaggrcccctgctcagwttyttggnwtctt

SEQ ID NO: 49 - DNA, 31 bp, synthetic construct
  misc_feature 1..31: note = VL sense primer MmLKd2
  source 1..31: mol_type = other DNA
atgggcwtcaagatgragtcacakwyycwgg

SEQ ID NO: 50 - DNA, 29 bp, synthetic construct
  misc_feature 1..29: note = VL sense primer MmLKe1
  source 1..29: mol_type = other DNA
atgagtgtgcycactcaggtcctggsgtt

SEQ ID NO: 51 - DNA, 31 bp, synthetic construct
  misc_feature 1..31: note = VL sense primer MmLKe2
  source 1..31: mol_type = other DNA
atgttgggaycgktttyammcttttcaattg

SEQ ID NO: 52 - DNA, 28 bp, synthetic construct
  misc_feature 1..28: note = VL sense primer MmLKe3
  source 1..28: mol_type = other DNA
atggaagccccagctcagcttctcttcc

SEQ ID NO: 53 - DNA, 26 bp, synthetic construct
  misc_feature 1..26: note = VL sense primer MmLKf1
  misc_feature 6: note = n= I
  misc_feature 12
  misc_feature 12: note = n is a, c, g, or t
  misc_feature 18: note = n=I
  source 1..26: mol_type = other DNA
atgagnmmktcnmttcanttcytggg

SEQ ID NO: 54 - DNA, 26 bp, synthetic construct
  misc_feature 1..26: note = VL sense primer MmLKf2
  misc_feature 12: note = n=I
  misc_feature 24: note = n=I
  source 1..26: mol_type = other DNA
atgakgthcycngctcagytyctnrg

SEQ ID NO: 55 - DNA, 25 bp, synthetic construct
  misc_feature 1..25: note = VL sense primer MmLKf3
  source 1..25: mol_type = other DNA
atggtrtccwcasctcagttccttg

SEQ ID NO: 56 - DNA, 27 bp, synthetic construct
  misc_feature 1..27: note = VL sense primer MmLKf4
  source 1..27: mol_type = other DNA
atgtatatatgtttgttgtctatttct

SEQ ID NO: 57 - DNA, 29 bp, synthetic construct
  misc_feature 1..29: note = VL sense primer MmLKg1
  source 1..29: mol_type = other DNA
atgaagttgcctgttaggctgttggtgct

SEQ ID NO: 58 - DNA, 29 bp, synthetic construct
  misc_feature 1..29: note = VL sense primer MmLKg2
  source 1..29: mol_type = other DNA
atggatttwcargtgcagattwtcagctt

SEQ ID NO: 59 - DNA, 27 bp, synthetic construct
  misc_feature 1..27: note = VL sense primer MmLKg3
  source 1..27: mol_type = other DNA
atggtyctyatvtccttgctgttctgg

SEQ ID NO: 60 - DNA, 27 bp, synthetic construct
  misc_feature 1..27: note = VL sense primer MmLKg4
  source 1..27: mol_type = other DNA
atggtyctyatvttrctgctgctatgg

SEQ ID NO: 61 - DNA, 20 bp, synthetic construct
  misc_feature 1..20: note = VL sense primer LL_AG_1
  source 1..20: mol_type = other DNA
attgctcaggttctttctcc

SEQ ID NO: 62 - DNA, 20 bp, synthetic construct
  misc_feature 1..20: note = VL sense primer LL_AG_2
  source 1..20: mol_type = other DNA
cagtcataatgtccagagga

SEQ ID NO: 63 - DNA, 20 bp, synthetic construct
  misc_feature 1..20: note = VL sense primer LL_AG_3
  source 1..20: mol_type = other DNA
cagtcataatgtccagggga

SEQ ID NO: 64 - DNA, 20 bp, synthetic construct
  misc_feature 1..20: note = VL sense primer LL_AG_4
  source 1..20: mol_type = other DNA
cagtcatactattcagagga

SEQ ID NO: 65 - DNA, 20 bp, synthetic construct
  misc_feature 1..20: note = VL sense primer LL_AG_5
  source 1..20: mol_type = other DNA
cagtcatagtgtctaatgga

SEQ ID NO: 66 - DNA, 20 bp, synthetic construct
  misc_feature 1..20: note = VL sense primer LL_AG_6
  source 1..20: mol_type = other DNA
cagtcatattgaccaatgga

SEQ ID NO: 67 - DNA, 20 bp, synthetic construct
  misc_feature 1..20: note = VL sense primer LL_AG_7
  source 1..20: mol_type = other DNA
cagtcatattgtccagtgga

SEQ ID NO: 68 - DNA, 20 bp, synthetic construct
  misc_feature 1..20: note = VL sense primer LL_AG_8
  source 1..20: mol_type = other DNA
cggtatctggtacctgtgga

SEQ ID NO: 69 - DNA, 20 bp, synthetic construct
  misc_feature 1..20: note = VL sense primer LL_AG_9
  source 1..20: mol_type = other DNA
cgagtccagcctcaagcagt

SEQ ID NO: 70 - DNA, 20 bp, synthetic construct
  misc_feature 1..20: note = VL sense primer LL_AG_10
  source 1..20: mol_type = other DNA
gaatcacaggcataatatgt

SEQ ID NO: 71 - DNA, 20 bp, synthetic construct
  misc_feature 1..20: note = VL sense primer LL_AG_11
  source 1..20: mol_type = other DNA
gaatcccaggcatgatatgt

SEQ ID NO: 72 - DNA, 20 bp, synthetic construct
  misc_feature 1..20: note = VL sense primer LL_AG_12
  source 1..20: mol_type = other DNA
gcatgtctggtgcctgtgca

SEQ ID NO: 73 - DNA, 20 bp, synthetic construct
  misc_feature 1..20: note = VL sense primer LL_AG_13
  source 1..20: mol_type = other DNA
ggaccacggtctcagctgtc

SEQ ID NO: 74 - DNA, 20 bp, synthetic construct
  misc_feature 1..20: note = VL sense primer LL_AG_14
  source 1..20: mol_type = other DNA
ggacttcagcctccagatgt

SEQ ID NO: 75 - DNA, 20 bp, synthetic construct
  misc_feature 1..20: note = VL sense primer LL_AG_15
  source 1..20: mol_type = other DNA
ggatatcaggtgcccagtgt

SEQ ID NO: 76 - DNA, 20 bp, synthetic construct
  misc_feature 1..20: note = VL sense primer LL_AG_16
  source 1..20: mol_type = other DNA
ggatccctggagccactggg

SEQ ID NO: 77 - DNA, 20 bp, synthetic construct
  misc_feature 1..20: note = VL sense primer LL_AG_17
  source 1..20: mol_type = other DNA
ggatccctggatccactgca

SEQ ID NO: 78 - DNA, 20 bp, synthetic construct
  misc_feature 1..20: note = VL sense primer LL_AG_18
  source 1..20: mol_type = other DNA
ggatctctggagtcagtggg

SEQ ID NO: 79 - DNA, 20 bp, synthetic construct
  misc_feature 1..20: note = VL sense primer LL_AG_19
  source 1..20: mol_type = other DNA
ggattcaggaaaccaacggt

SEQ ID NO: 80 - DNA, 20 bp, synthetic construct
  misc_feature 1..20: note = VL sense primer LL_AG_20
  source 1..20: mol_type = other DNA
ggattccagcctccagaggt

SEQ ID NO: 81 - DNA, 20 bp, synthetic construct
  misc_feature 1..20: note = VL sense primer LL_AG_21
  source 1..20: mol_type = other DNA
ggattcctgcttccagcagt

SEQ ID NO: 82 - DNA, 20 bp, synthetic construct
  misc_feature 1..20: note = VL sense primer LL_AG_22
  source 1..20: mol_type = other DNA
ggattcgggaaaccaacggt

SEQ ID NO: 83 - DNA, 20 bp, synthetic construct
  misc_feature 1..20: note = VL sense primer LL_AG_23
  source 1..20: mol_type = other DNA
ggatttcagcctccacaggt

SEQ ID NO: 84 - DNA, 20 bp, synthetic construct
  misc_feature 1..20: note = VL sense primer LL_AG_24
  source 1..20: mol_type = other DNA
ggctccaaggcatgagctgt

SEQ ID NO: 85 - DNA, 20 bp, synthetic construct
  misc_feature 1..20: note = VL sense primer LL_AG_25
  source 1..20: mol_type = other DNA
ggcttcatggtgctcagtgt

SEQ ID NO: 86 - DNA, 20 bp, synthetic construct
  misc_feature 1..20: note = VL sense primer LL_AG_26
  source 1..20: mol_type = other DNA
ggcttacagacgcaggatgt

SEQ ID NO: 87 - DNA, 20 bp, synthetic construct
  misc_feature 1..20: note = VL sense primer LL_AG_27
  source 1..20: mol_type = other DNA
ggcttacagatgccagatgt

SEQ ID NO: 88 - DNA, 20 bp, synthetic construct
  misc_feature 1..20: note = VL sense primer LL_AG_28
  source 1..20: mol_type = other DNA
gggtatctggtacctgtggg

SEQ ID NO: 89 - DNA, 20 bp, synthetic construct
  misc_feature 1..20: note = VL sense primer LL_AG_29
  source 1..20: mol_type = other DNA
gggtatctggtgcctgtgca

SEQ ID NO: 90 - DNA, 20 bp, synthetic construct
  misc_feature 1..20: note = VL sense primer LL_AG_30
  source 1..20: mol_type = other DNA
gggttccaggttccactggt

SEQ ID NO: 91 - DNA, 20 bp, synthetic construct
  misc_feature 1..20: note = VL sense primer LL_AG_31
  source 1..20: mol_type = other DNA
ggttatatggtgctgatggg

SEQ ID NO: 92 - DNA, 20 bp, synthetic construct
  misc_feature 1..20: note = VL sense primer LL_AG_32
  source 1..20: mol_type = other DNA
ggttcccaggtgccagatgt

SEQ ID NO: 93 - DNA, 20 bp, synthetic construct
  misc_feature 1..20: note = VL sense primer LL_AG_33
  source 1..20: mol_type = other DNA
ggttgtctggtgttgaagga

SEQ ID NO: 94 - DNA, 20 bp, synthetic construct
  misc_feature 1..20: note = VL sense primer LL_AG_34
  source 1..20: mol_type = other DNA
ggtttccaggtatcagatgt

SEQ ID NO: 95 - DNA, 20 bp, synthetic construct
  misc_feature 1..20: note = VL sense primer LL_AG_35
  source 1..20: mol_type = other DNA
ggtttccaggtgcaagatgt

SEQ ID NO: 96 - DNA, 20 bp, synthetic construct
  misc_feature 1..20: note = VL sense primer LL_AG_36
  source 1..20: mol_type = other DNA
ggtttgcaggtggtaaatgt

SEQ ID NO: 97 - DNA, 20 bp, synthetic construct
  misc_feature 1..20: note = VL sense primer LL_AG_37
  source 1..20: mol_type = other DNA
ggtttttaggtgccagatgt

SEQ ID NO: 98 - DNA, 20 bp, synthetic construct
  misc_feature 1..20: note = VL sense primer LL_AG_38
  source 1..20: mol_type = other DNA
gtgtcacagtgtcaaaggga

SEQ ID NO: 99 - DNA, 20 bp, synthetic construct
  misc_feature 1..20: note = VL sense primer LL_AG_39
  source 1..20: mol_type = other DNA
gtgtctctgattctagggca

SEQ ID NO: 100 - DNA, 20 bp, synthetic construct
  misc_feature 1..20: note = VL sense primer LL_AG_40
  source 1..20: mol_type = other DNA
gtgtgtctggtgctcatggg

SEQ ID NO: 101 - DNA, 20 bp, synthetic construct
  misc_feature 1..20: note = VL sense primer LL_AG_41
  source 1..20: mol_type = other DNA
gttttcaaggtaccagatat

SEQ ID NO: 102 - DNA, 20 bp, synthetic construct
  misc_feature 1..20: note = VL sense primer LL_AG_42
  source 1..20: mol_type = other DNA
gttttcaaggtaccagatgt

SEQ ID NO: 103 - DNA, 65 bp, synthetic construct
  misc_feature 1..65: note = Illumina Index Antisense primer
  misc_feature 25..31: note = n = a or g or c or t/u or other modified nucleotide
  source 1..65: mol_type = other DNA
caagcagaagacggcatacgagatnnnnnnngtgactggagttcagacgtgtgctcttccgatct

SEQ ID NO: 104 - DNA, 78 bp, synthetic construct
  misc_feature 1..78: note = VH sense primer VHs1
  source 1..78: mol_type = other DNA
aatgatacggcgaccaccgagatctacactctttccctacacgacgctcttccgatctgatgtgaagcttcaggagtc

SEQ ID NO: 105 - DNA, 78 bp, synthetic construct
  misc_feature 1..78: note = VH sense primer VHs2
  source 1..78: mol_type = other DNA
aatgatacggcgaccaccgagatctacactctttccctacacgacgctcttccgatctcaggtgcagctgaaggagtc

SEQ ID NO: 106 - DNA, 78 bp, synthetic construct
  misc_feature 1..78: note = VH sense primer VHs3
  source 1..78: mol_type = other DNA
aatgatacggcgaccaccgagatctacactctttccctacacgacgctcttccgatctcaggtgcagctgaagcagtc

SEQ ID NO: 107 - DNA, 78 bp, synthetic construct
  misc_feature 1..78: note = VH sense primer VHs4
  source 1..78: mol_type = other DNA
aatgatacggcgaccaccgagatctacactctttccctacacgacgctcttccgatctcaggttactctgaaagagtc

SEQ ID NO: 108 - DNA, 79 bp, synthetic construct
  misc_feature 1..79: note = VH sense primer VHs5
  source 1..79: mol_type = other DNA
aatgatacggcgaccaccgagatctacactctttccctacacgacgctcttccgatctgaggtccagctgcaacaatct

SEQ ID NO: 109 - DNA, 78 bp, synthetic construct
  misc_feature 1..78: note = VH sense primer VHs6
  source 1..78: mol_type = other DNA
aatgatacggcgaccaccgagatctacactctttccctacacgacgctcttccgatctgaggtccagctgcagcagtc

SEQ ID NO: 110 - DNA, 79 bp, synthetic construct
  misc_feature 1..79: note = VH sense primer VHs7
  source 1..79: mol_type = other DNA
aatgatacggcgaccaccgagatctacactctttccctacacgacgctcttccgatctcaggtccaactgcagcagcct

SEQ ID NO: 111 - DNA, 78 bp, synthetic construct
  misc_feature 1..78: note = VH sense primer VHs8
  source 1..78: mol_type = other DNA
aatgatacggcgaccaccgagatctacactctttccctacacgacgctcttccgatctgaggtgaagctggtggagtc

SEQ ID NO: 112 - DNA, 78 bp, synthetic construct
  misc_feature 1..78: note = VH sense primer VHs9
  source 1..78: mol_type = other DNA
aatgatacggcgaccaccgagatctacactctttccctacacgacgctcttccgatctgaggtgaagctggtggaatc

SEQ ID NO: 113 - DNA, 78 bp, synthetic construct
  misc_feature 1..78: note = VH sense primer VHs10
  source 1..78: mol_type = other DNA
aatgatacggcgaccaccgagatctacactctttccctacacgacgctcttccgatctgatgtgaacttggaagtgtc

SEQ ID NO: 114 - DNA, 78 bp, synthetic construct
  misc_feature 1..78: note = VH sense primer VHs11
  source 1..78: mol_type = other DNA
aatgatacggcgaccaccgagatctacactctttccctacacgacgctcttccgatctgaggtccagctgcaacagtc

SEQ ID NO: 115 - DNA, 78 bp, synthetic construct
  misc_feature 1..78: note = VH sense primer VHs12
  source 1..78: mol_type = other DNA
aatgatacggcgaccaccgagatctacactctttccctacacgacgctcttccgatctgaggtgcagctggaggagtc

SEQ ID NO: 116 - DNA, 79 bp, synthetic construct
  misc_feature 1..79: note = VL sense primer VKs1
  source 1..79: mol_type = other DNA
aatgatacggcgaccaccgagatctacactctttccctacacgacgctcttccgatctgatattgtgatgacgcaggct

SEQ ID NO: 117 - DNA, 76 bp, synthetic construct
  misc_feature 1..76: note = VL sense primer VKs2
  source 1..76: mol_type = other DNA
aatgatacggcgaccaccgagatctacactctttccctacacgacgctcttccgatctgatattgtgataacccag

SEQ ID NO: 118 - DNA, 79 bp, synthetic construct
  misc_feature 1..79: note = VL sense primer VKs3
  source 1..79: mol_type = other DNA
aatgatacggcgaccaccgagatctacactctttccctacacgacgctcttccgatctgacattgtgctgacccaatct

SEQ ID NO: 119 - DNA, 79 bp, synthetic construct
  misc_feature 1..79: note = VL sense primer VKs4
  source 1..79: mol_type = other DNA
aatgatacggcgaccaccgagatctacactctttccctacacgacgctcttccgatctgacattgtgatgacccagtct

SEQ ID NO: 120 - DNA, 79 bp, synthetic construct
  misc_feature 1..79: note = VL sense primer VKs5
  source 1..79: mol_type = other DNA
aatgatacggcgaccaccgagatctacactctttccctacacgacgctcttccgatctgatattgtgctaactcagtct

SEQ ID NO: 121 - DNA, 79 bp, synthetic construct
  misc_feature 1..79: note = VL sense primer VKs6
  source 1..79: mol_type = other DNA
aatgatacggcgaccaccgagatctacactctttccctacacgacgctcttccgatctgatatccagatgacacagact

SEQ ID NO: 122 - DNA, 79 bp, synthetic construct
  misc_feature 1..79: note = VL sense primer VKs7
  source 1..79: mol_type = other DNA
aatgatacggcgaccaccgagatctacactctttccctacacgacgctcttccgatctgacatccagctgactcagtct

SEQ ID NO: 123 - DNA, 79 bp, synthetic construct
  misc_feature 1..79: note = VL sense primer VKs8
  source 1..79: mol_type = other DNA
aatgatacggcgaccaccgagatctacactctttccctacacgacgctcttccgatctcaaattgttctcacccagtct

SEQ ID NO: 124 - DNA, 79 bp, synthetic construct
  misc_feature 1..79: note = VL sense primer VKs9
  source 1..79: mol_type = other DNA
aatgatacggcgaccaccgagatctacactctttccctacacgacgctcttccgatctgatgttttgatgacccaaact

SEQ ID NO: 125 - DNA, 20 bp, synthetic construct
  misc_feature 1..20: note = primer RT_VLk
  source 1..20: mol_type = other DNA
gatggtgggaagatggatac

SEQ ID NO: 126 - DNA, 58 bp, synthetic construct
  misc_feature 1..58: note = sense primer used in PCR3
  source 1..58: mol_type = other DNA
aatgatacggcgaccaccgagatctacactctttccctacacgacgctcttccgatct

SEQ ID NO: 127 - DNA, 24 bp, synthetic construct
  misc_feature 1..24: note = antisense primer used in PCR3
  source 1..24: mol_type = other DNA
caagcagaagacggcatacgagat

SEQ ID NO: 128 - protein, 21 aa, synthetic construct
  REGION 1..21: note = CDR3
CARDWSRSWYLAPNGPDLDYW

SEQ ID NO: 129 - protein, 12 aa, synthetic construct [sequence omitted]

SEQ ID NO: 130 - protein, 18 aa, synthetic construct
  REGION 1..18: note = CDR3
CARGGKSDDGNFRYFDHW

SEQ ID NO: 131 - protein, 13 aa, synthetic construct
  REGION 1..13: note = CDR3
CQQRSSWPPGWTF

SEQ ID NO: 132 - protein, 20 aa, synthetic construct
  REGION 1..20: note = CDR3
CAKSFGFGGVIVIGGYFLHW

SEQ ID NO: 133 - protein, 11 aa, synthetic construct [sequence omitted]

SEQ ID NO: 134 - protein, 17 aa, synthetic construct
  REGION 1..17: note = CDR3
CARHKTTSGWYSPLDYW

SEQ ID NO: 135 - protein, 11 aa, synthetic construct
  REGION 1..11: note = CDR3
CQQYSGSVWTF

SEQ ID NO: 136 - protein, 18 aa, synthetic construct
  REGION 1..18: note = CDR3
CARGVKAAGRTPNWFGPW

SEQ ID NO: 137 - protein, 14 aa, synthetic construct [sequence omitted]

SEQ ID NO: 138 - protein, 17 aa, synthetic construct
  REGION 1..17: note = CDR3
CAREVSADILTGYYDYW

SEQ ID NO: 139 - protein, 11 aa, synthetic construct [sequence omitted]